(19) United States
(12) Patent Application Publication
(10) Pub. No.: US 2008/0242622 A1
(43) Pub. Date: Oct. 2, 2008

(54) Identification of genetic alterations that modulate drug sensitivity in cancer treatments

(75) Inventors: Scott W. Lowe, Cold Spring Harbor, NY (US); Michael Hemann, Cambridge, MA (US); Gregory J. Hannon, Huntington, NY (US); Darren Burgess, Surrey (GB)

Correspondence Address: ROPES & GRAY LLP, PATENT DOCKETING 39/41, ONE INTERNATIONAL PLACE, BOSTON, MA, 02110-2624, US

(73) Assignee: Cold Spring Harbor Laboratory, Cold Spring Harbor, NY (US)

(21) Appl. No.: 12/077,737

(22) Filed: Mar. 19, 2008

Related U.S. Application Data
(60) Provisional application No. 60/918,962, filed on Mar. 19, 2007.

(51) Int. Cl. A61K 31/7028; C40B 30/06; C12Q 1/68; A61K 31/7105; A61K 31/704; C12Q 1/02

(52) U.S. Cl. 514/27; 506/10; 435/6; 435/29; 514/34; 514/44

(57) ABSTRACT

This invention features methods of identifying genetic alterations that can modulate cancer cells' sensitivity to an anti-cancer drug. Information on such genetic alterations can be used to predict cancer therapeutic outcomes and to stratify patient populations to maximize therapeutic efficacy.

### CROSS REFERENCE TO RELATED APPLICATIONS

This application claims the benefit of U.S. Provisional Application Ser. No. 60/918,962, filed Mar. 19, 2007, which is incorporated by reference in its entirety.

### GOVERNMENT SUPPORT

The work described herein was funded, in whole or in part, by Grant Number CA13106 from the National Cancer Institute. The United States government may have certain rights in the invention.

### FIELD OF THE INVENTION

This invention relates to the use of RNA interference (RNAi) technology to identify genetic alterations that modulate cancer cells' sensitivity to chemotherapeutics.

### BACKGROUND INFORMATION

Cancer is the second leading cause of death in industrial countries. Many cancers show initial or compulsory chemo-resistance. Resistance to cytotoxic agents used in cancer therapy remains a major obstacle in the treatment of human malignancies. Since most anti-cancer agents were discovered through empirical screens, efforts to overcome resistance are hindered by our limited understanding of why these agents are effective. Furthermore, although cancer usually arises from a combination of mutations in oncogenes and tumor suppressor genes, the mechanisms by which genetic mutations result in tumorigenesis or resistance to cytotoxic agents are poorly understood.

The majority of chemotherapeutic drugs can be divided into several categories, including alkylating agents, anti-metabolites, anthracyclines, plant alkaloids, topoisomerase poisons, and monoclonal antibodies.

Topoisomerases are cellular enzymes essential for maintaining the topology of DNA. Type I topoisomerases function by nicking one of the strands of the DNA double helix, twisting it around the other strand, and re-ligating the nicked strand. Type I enzymes can be further subdivided into type IA and type IB, based on the chemistry of their action. Type IA topoisomerases (such as TOP3A, TOP3B) change the linking number of a circular DNA strand strictly by units of 1, whereas Type IB topoisomerases (such as TOP1) change the linking number by multiples of 1. Type II topoisomerases cut both strands of the DNA helix simultaneously. Once cut, the ends of the DNA are separated, and a second DNA duplex is passed through the break. Following passage, the cut DNA is resealed. This reaction allows type II topoisomerases to increase or decrease the linking number of a DNA loop by 2 units, and promotes chromosome disentanglement. There are two subclasses of type II topoisomerases, type IIA and IIB. Type IIA topoisomerases include the enzymes DNA gyrase, eukaryotic topoisomerase II (such as TOP2A and TOP2B), and bacterial topoisomerase IV. Type IIB topoisomerases are structurally and biochemically distinct, and comprise a single family member, topoisomerase VI. Type IIB topoisomerases are found in archaea and some higher plants. Type III DNA topoisomerase was first identified by studying the hyper-recombination and slow growth phenotypes of yeast mutants. Topoisomerase III interacts with DNA helicase SGS1 and the two proteins are involved in DNA recombination, cellular aging and maintenance of genome stability.

TOP1 and TOP2 poisons interfere with both DNA transcription and replication by upsetting proper DNA supercoiling. TOP1 and TOP2 poisons alter the activity of the topoisomerases by stabilizing the DNA-topoisomerase complex (i.e., DNA molecule is cleaved and covalently-attached to the topoisomerase, but not re-ligated). Commonly used type I topoisomerase poisons include camptothecin, irinotecan and topotecan. Examples of type II poisons include doxorubicin (Adriamycin), amsacrine, etoposide, and teniposide, all of which primarily target TOP2A. There are no known drugs targeting type III topoisomerase enzymes.

Chemotherapy is physically exhausting for a patient. Current chemotherapeutic regimens have a range of side effects, mainly affecting fast-dividing cells of the body. Virtually all chemotherapeutic regimens suppress the immune system by attacking hematopoietic cells in the bone marrow. This leads to a decrease of white blood cells, red blood cells, and platelets. Other common side-effects include hair loss (alopecia), nausea and vomiting, diarrhea or constipation, and hemorrhage.

Doxorubicin (Adriamycin) is an anthracycline DNA damaging agent that exerts its effects primarily by targeting of the topoisomerase 2 activity and DNA intercalation. Along with etoposide and the camptothecin derivatives, doxorubicin is one of several topoisomerase-targeted drugs currently used as front-line therapies for a wide variety of cancers. For example, doxorubicin is widely used to treat Hodgkin lymphoma, breast cancer, lung cancer, soft tissue sarcoma, Kahler's disease (multiple myeloma), and recurring instances of ovarian cancer. Doxorubicin acts by stabilizing the Topoisomerase II complex after TOP2 breaks the DNA chain for replication, preventing the DNA double helix from annealing and thereby stopping the replication process. Acute side-effects of doxorubicin include nausea, vomiting, and heart arrhythmia. Doxorubicin can also cause a decrease in white blood cells, and hair loss. When the cumulative dose of doxorubicin reaches 550 mg/m², the risks of developing cardiac side effects, including congestive heart failure, dilated cardiomyopathy, and death, dramatically increase.

A myriad of genetic factors influence the efficacy of cancer chemotherapy, including both somatic changes in the tumor itself as well as genetic polymorphisms present in the patient. These factors include: increased expression of detoxification pumps that prevent access of the drug to its target, point mutations that disrupt the drug-target interaction, and mutations in stress response pathways (e.g. p53 loss). In order to tailor treatment successfully to the individual patient, a more complete understanding of the genetic determinants of therapy response is necessary.

Therefore, there is an urgent need to develop a safe, efficient method of determining the pharmacology, toxicity, and effectiveness of chemotherapeutic drugs in cancer patients. Patient stratification allows clinicians to provide a treatment regimen based on tumor-specific genetic modifications, and to predict the likely response of an individual to a therapeutic treatment.

### SUMMARY OF THE INVENTION

The present invention provides methods and compositions useful in identification of genetic alterations that lead to chemotherapeutic agent resistance or sensitization, identification of therapeutic targets for chemotherapy of cancerous cells, identification of cancer patients that may benefit from a particular treatment regimen, and identification of novel chemotherapeutic compounds that enhance the effectiveness of a chemotherapy regimen.

The present invention is based on the discovery that certain genetic alterations can modulate cancer cells' sensitivity to an anti-cancer drug. Information on such genetic alterations can be used to predict cancer therapeutic outcomes and to stratify patient populations to maximize therapeutic efficacy.

In one aspect, the present invention provides a method for identifying a gene whose down-regulation in a cancer cell results in the cancer cell's resistance to a chemotherapeutic agent, comprising: (a) providing a library of RNA interference (RNAi) molecules, wherein each of RNAi molecules inhibits the expression of a target mammalian gene; (b) transfecting a plurality of mammalian cells with the library and expressing the RNAi molecules in the transfected mammalian cells; (c) treating the transfected cells with the chemotherapeutic agent; and (d) identifying an RNAi molecule that increases the survival of a transfected cell, as compared to cells that do not express the RNAi molecule. AN RNAi molecule that increases the survival of a transfected cell indicates that the down-regulation its target gene results in the cancer cell's resistance to the chemotherapeutic agent. Conversely, an RNAi molecule that reduces the survival of a transfected cell, as compared to cells that do not express the RNAi molecule, indicates that the up-regulation its target gene results in the cancer cell's resistance to the chemotherapeutic agent. The RNAi molecules may inhibit the expression of genes that are known to be up-regulated or down-regulated in human cancers. Alternatively, the RNAi molecules may inhibit expression of genes whose functions remain unknown.

In certain embodiments, the chemotherapeutic agent targets topoisomerase 1 (TOP1), such as camptothecin or irinotecan. In certain embodiments, the chemotherapeutic agent targets topoisomerase 2A (TOP2A), such as doxorubincin.

In certain embodiments, the RNAi molecule is identified using polymerase chain reaction (PCR). In certain embodiments, the RNAi molecule is identified using microarray.

In another aspect, the present invention provides a method to identify a gene whose up-regulation or down-regulation in a cancer cell results in the cancer cell's sensitivity to a chemotherapeutic agent, comprising: (a) providing an RNA interference (RNAi) molecule that inhibits the expression of a candidate gene; (b) transfecting a plurality of mammalian cells with the RNAi molecule; (c) treating the transfected cells with the chemotherapeutic agent; (d) monitoring survival of the transfected cells and control cells that do not express the RNAi molecule. AN RNAi molecule that reduces the survival of a transfected cell, as compared to cells that do not express the RNAi molecule, indicates that the down-regulation of its target gene enhances the cancer cell's sensitivity to the chemotherapeutic agent. Conversely, an RNAi molecule that enhances the survival of a transfected cell indicates that the up-regulation of its target gene may result in the cancer cell's sensitivity to the chemotherapeutic agent.

In certain embodiments, the chemotherapeutic agent targets TOP1. In some embodiments, the chemotherapeutic agent is a TOP1 inhibitor. In certain embodiments, the chemotherapeutic agent is a TOP1 poison. In certain embodiments, the chemotherapeutic agent targets TOP2, in particular, TOP2A. In certain embodiments, the chemotherapeutic agent is a TOP2 inhibitor. In certain embodiments, the chemotherapeutic agent is a TOP2 poison.

In another aspect, the present invention provides a method to identify a gene whose up-regulation or down-regulation in a cancer cell results in the cancer cell's sensitivity to a chemotherapeutic agent, comprising: (a) providing a library of RNA interference (RNAi) molecules, wherein each of RNAi molecules inhibits the expression of a target mammalian gene; (b) transfecting a plurality of mammalian cells with the library and expressing the RNAi molecules in the transfected mammalian cells; (c) treating the transfected cells with the chemotherapeutic agent; and (d) identifying an RNAi molecule that decreases the survival of a transfected cell, as compared to cells that do not express the RNAi molecule. AN RNAi molecule that decreases the survival of a transfected cell indicates that the down-regulation its target gene results in the cancer cell's sensitivity to the chemotherapeutic agent. Conversely, an RNAi molecule that increases the survival of a transfected cell, as compared to cells that do not express the RNAi molecule, indicates that the up-regulation its target gene results in the cancer cell's sensitivity to the chemotherapeutic agent.

In certain embodiments, the chemotherapeutic agent targets TOP1. In some embodiments, the chemotherapeutic agent is a TOP1 inhibitor. In certain embodiments, the chemotherapeutic agent is a TOP1 poison. In certain embodiments, the chemotherapeutic agent targets TOP2, in particular, TOP2A. In certain embodiments, the chemotherapeutic agent is a TOP2 inhibitor. In certain embodiments, the chemotherapeutic agent is a TOP2 poison. In certain embodiment, the target gene is identified using microarray.

In another aspect, the present invention discloses a method for identifying an agent that enhances the effectiveness of a treatment with a TOP2-trageting chemotherapeutic agent, comprising (a) contacting a mammalian cell with the candidate agent, and (b) comparing the expression or activity level of TOP1 of the treated cells to a control (for example, cells not treated with the candidate agent). A decreased expression level of TOP1 in the presence of the agent, as compared to control, may indicate that the candidate agent is a TOP1 inhibitor and may be used in conjunction with a TOP2-targeting cancer drug to enhance to effectiveness of the TOP2-targeting drug. In certain embodiments, the TOP2-targeting therapeutic agent is a TOP2 poison. In certain embodiments, the TOP2 poison is a TOP2A poison.

In another aspect, the present invention discloses a method for identifying an agent that enhances the effectiveness of a treatment with a TOP2-trageting chemotherapeutic agent, comprising (a) contacting a mammalian cell with the candidate agent, and (b) comparing the expression or activity level of Bmi1 of the treated cells to a control (for example, cells not treated with the candidate agent). A decreased expression level of Bmi1 in the presence of the agent, as compared to control, may indicate that the candidate agent is a Bmi1 inhibitor and may be used in conjunction with a TOP2-targeting cancer drug to enhance to effectiveness of the TOP2-targeting drug. In certain embodiments, the TOP2-targeting therapeutic agent is a TOP2 poison. In certain embodiments, the TOP2 poison is a TOP2A poison.

In another aspect, the present invention provides a method for identifying a cancer patient who may benefit from a treatment with a TOP2-targeting chemotherapeutic agent, comprising: (a) obtaining a cancer cell from the patient; (b) determining the expression or activity level of TOP1 in the cancer cell. A decrease in the TOP1 expression or activity level in the cancer cell as compared to a control indicates that the patient may benefit from a treatment with a TOP2-targeting chemotherapeutic agent.

In another aspect, the present invention provides a method for identifying a cancer patient who may benefit from a treatment with a TOP2-targeting chemotherapeutic agent, comprising: (a) obtaining a cancer cell from the patient; (b) determining the expression or activity level of Bmi1 in the cancer cell. A decrease in the Bmi1 expression or activity level in the cancer cell as compared to a control indicates that the patient may benefit from a treatment with a TOP2-targeting chemotherapeutic agent.

In certain embodiments, the cancer cell is from bladder cancer, breast cancer, colon cancer, kidney cancer, liver cancer, lung cancer, esophagus cancer, gall bladder cancer, ovarian cancer, pancreas cancer, stomach cancer, cervical cancer, thyroid cancer, prostate cancer, skin cancer, leukemia, B-cell lymphoma, T-cell lymphoma, Hodgkins lymphoma, non-Hodgkins lymphoma, hairy cell lymphoma, Burkett's lymphoma, fibrosarcoma, rhabdomyosarcoma, astrocytoma, neuroblastoma, glioma and schwannomas, melanoma, seminoma, teratocarcinoma, osteosarcoma, xenoderoma pigmentosum, keratoctanthoma, thyroid follicular cancer, or Kaposi's sarcoma. In certain embodiment, the cancer cell is from acute myelogenous leukemia.

In another aspect, the present invention provides a method for treating a cancer patient identified by the method described above, by administering to those patient a TOP2-targeting chemotherapeutic agent.

In another aspect, the present invention provides a method for treating a cancer patient, comprising administering to the patient a TOP1 inhibitor (such as an RNAi molecule, including an shRNA molecule) that down-regulates the expression or activity of TOP1, and a TOP2A-targeting chemotherapeutic agent (such as doxorubicin, etoposide, mitoxantrone, mAMSA, amonafide, batracylin or menadione).

In another aspect, the present invention provides a method for treating a cancer patient, comprising administering to the patient a Bmi1 inhibitor that down-regulates the expression or activity of Bmi1 (such as an RNAi molecule, including an shRNA molecule), and a TOP2A-targeting chemotherapeutic agent (such as doxorubicin, etoposide, mitoxantrone, mAMSA, amonafide, batracylin or menadione).

In certain embodiments, the RNAi is part of a viral vector, such as an adenoviral, lentiviral, or retroviral vector. In certain embodiments, the RNAi molecule is administered systemically in a pharmaceutical preparation.

In another aspect, the present invention provides a method for inhibiting a cancer cell growth, comprising contacting the cancer cell with a TOP1 inhibitor (such as an RNAi molecule, including an shRNA molecule) that down-regulates the expression or activity of TOP1, and a TOP2A-targeting chemotherapeutic agent (such as doxorubicin, etoposide, mitoxantrone, mAMSA, amonafide, batracylin or menadione).

In another aspect, the present invention provides a method for inhibiting a cancer cell growth, comprising contacting the cancer cell with a Bmi1 inhibitor (such as an RNAi molecule, including an shRNA molecule) that down-regulates the expression or activity of Bmi1, and a TOP2A-targeting chemotherapeutic agent (such as doxorubicin, etoposide, mitoxantrone, mAMSA, amonafide, batracylin or menadione).

In certain embodiments, the RNAi is part of a viral vector, such as an adenoviral, lentiviral, or retroviral vector. In certain embodiments, the RNAi molecule is administered systemically in a pharmaceutical preparation.

The methods disclosed in the present invention may be used to design a rational therapy, or select patient populations for the purposes of clinical trials. The method may be used to identify one or more genes that are linked to drug-resistance of any known chemotherapeutic drugs. The expression profile of those genes in a patient may be used to predict whether the patients will likely respond to a particular cancer treatment.

### BRIEF DESCRIPTION OF THE DRAWINGS

FIGS. 1A-C show the subcloning of the “Cancer 1000” library using bacterial mating method. FIG. 1A shows an overview of the Mating-Assisted Genetically Integrated Cloning (MAGIC) system (adapted from Li and Elledge, 2005). FIG. 1B is a schematic illustration of pSM2 vector (“Donor”) to MLP vector (“Recipient”) transfer. FIG. 1C is a photograph showing XhoI/AgeI diagnostic restriction digests of final clones. Arrows show the positions of correct fragments.

FIGS. 2A and 2B show the subcloning of the “Cancer 1000” library using a restriction enzyme cut-and-paste method. FIG. 2A is a schematic illustration of donor pSM2 (original vector) and recipient MLP and MLS vectors (Recipients). FIG. 2B is a table detailing the comparison of two cloning strategies (X=XhoI, R=EcoRI, S=SalI, M=MluI).

FIGS. 3A-C are schematic illustrations of shRNA screening strategies for doxorubicin resistance in Eμ-Myc; Arf^{-/-} lymphoma cells in vitro. FIG. 3A is a schematic illustration of a protocol using Cancer 1000 shRNA library, subdivided into pools of 48 or 96 shRNA complexity, with single treatment of doxorubicin. All pools were treated with doxorubicin (8 and 16 ng/ml). Pools scoring for GFP enrichment in the GFP competition assay, relative to untreated controls, were prioritized for genomic PCR amplification of shRNA integrants, subcloning and sequencing. FIG. 3B is a schematic illustration of a protocol using whole Cancer 1000 with single treatment of doxorubicin. Constituent shRNAs were sequenced (as above) in lymphoma cells surviving 16 ng/ml doxorubicin treatment. shRNA representation was compared across 6 biological replicates for commonly recovered shRNAs. FIG. 3C is a schematic illustration of a protocol using whole Cancer 1000 with serial treatments of doxorubicin. Following each of three doxorubicin treatments, constituent shRNA integrants were subcloned, serving as an “enriched” pool for the next retroviral transduction, ensuring that enrichment was for relevant shRNAs rather than cellular mutants (e.g. p53) that might become enriched by serial treatment alone. In all cases, after retroviral infection (day 0) cells were treated (day 1) for 24 hours with doxorubicin, followed by dilutions into fresh medium on days 2 and 5. Genomic DNA was prepared on day 5 for serial enrichment experiments and day 8 for the single treatment experiments.

FIGS. 4A-C summarize shRNA screening results. Three distinct screening strategies (FIG. 3) using genomic PCR and standard DNA sequencing for hairpin identification resulted in overlapping datasets. FIG. 4A summarizes pool-by-pool approach. shRNA pools (48 or 96 shRNA complexity) resulting in GFP enrichment upon doxorubicin treatment had their constituent shRNAs sequenced. Approximately 100 sequence reads were performed for treated versus untreated samples. Examples for pools 12 and 17 are shown in lower panels. Individual shRNAs that were enriched in treated samples, according to DNA sequencing data, were retested in the GFP competition assay and successful validations are indicated. FIG. 4B summarizes hairpin sequence reads from whole Cancer 1000 shRNA screen following single doxorubicin treatment. Of six biological replicates (˜100 sequence reads for each) hits were prioritized based on 1) multiple independent shRNAs sequenced targeting the same gene (left) or 2) individual shRNAs identified in multiple biological replicates (right). FIG. 4C summarizes results from whole Cancer 1000 with serial enrichment screening. Over three rounds of doxorubicin treatment and shRNA subcloning, the shRNA set became progressively enriched for shRNAs mediating doxorubicin resistance as indicated by the ability of the pool to score in the GFP competition assay (upper panel). Doxorubicin resistance-mediating shRNAs dominated the final enriched pool as shown by shRNA identification by DNA sequencing (lower panel).

FIGS. 5A-C show a rapid RNAi enrichment screening protocol to identify mediators of doxorubicin resistance. FIG. 3A is a schematic illustration of the competition assay. shRNAs (operably linked to GFP) modifying the response to drug treatment can provoke a therapy-dependent change in percentage GFP-containing cells (“GFP+”) in a mixed population of shRNA-transfected and uninfected cells. FIGS. 3B and 3C show that shRNAs identified by rapid RNAi enrichment screens were validated for doxorubicin (DXR) resistance, as shown by GFP competition assay (FIG. 3B), and knockdown expression of their intended target, as shown by Western blot analysis (FIG. 3C).

FIGS. 6A-E demonstrate that multiple independent Chk2 shRNAs caused chemotherapy resistance in Eμ-Myc; Arf^{-/-} lymphoma cells. FIGS. 6A and 6B are western blot analyses of six independent Chk2 shRNAs knocking down Chk2 expression, relative to controls. (Data in A is partially duplicated from FIG. 5C). FIG. 6C is a schematic representation of GFP competition assay data, illustrating the trends expected from shRNAs modulating chemotherapy response. FIGS. 6D and 6E show that Chk2 knockdown caused resistance to doxorubicin (D) and camptothecin (E) relative to the vector control, as shown by a survival advantage for shChk2 (GFP⁺) cells in the GFP competition assay, 24 hours after treatment. Additional Chk2-targeted shRNAs that did not significantly knock down Chk2 expression and were not cytoprotective in the same assays (data not shown).

FIGS. 7A-C demonstrate that multiple Top2A shRNAs caused resistance specifically to topoisomerase 2 poisons. FIG. 7A shows that Top2A knockdown (via 4 independent Top2A shRNAs) caused doxorubicin resistance in Eμ-Myc; Arf^{-/-} lymphoma cells in vitro, as shown by doxorubicin-mediated GFP enrichment in the GFP competition assay, 24 hours after treatment. FIG. 7B shows that shTop2A caused attenuated doxorubicin resistance in a p53 deficient background, as shown by GFP competition assay on Eμ-Myc; p53^{-/-} lymphoma cells treated for 24 hours at the indicated doxorubicin doses. FIG. 7C shows that Top2A knockdown caused resistance specifically to topoisomerase 2 poisons, as shown by GFP competition assay of Eμ-Myc; Arf^{-/-} lymphoma cells, 24 hours after treatment.

FIGS. 8A-D demonstrate that suppression of Top2A expression caused resistance to topoisomerase 2 poisons in vitro. FIGS. 8A and 8B are flow cytometric analyses of lymphoma cells expressing shTop2A 668 (A) or shp53 1224 (B) following 24 hours of the indicated drug treatments. FIG. 8C shows an in vitro viability analysis of doxorubicin-treated lymphoma cells. Lymphoma cells, transduced singly with four independent Top2A shRNAs, were puromycin selected and treated with doxorubicin for 24 hours at the indicated doses. Viability was assayed by flow cytometry (FSC versus SSC) and plotted relative to untreated controls. Error bars are ±SEM from 3 replicates. FIG. 8D shows immunoblotting of lymphoma cell lysates expressing no short hairpin (Vector), or Top1, Top2A or p53 shRNAs in the presence or absence of doxorubicin (DXR, 15.6 ng/ml for 8 hours).

FIGS. 9A-C demonstrate that Top2A knockdown resulted in diminished DNA damage and apoptosis upon doxorubicin treatment, relative to a vector control, as determined by γ-H2AX and activated caspase-3 immunofluorescence. FIG. 9A represents sample γ-H2AX immunofluorescence images. FIG. 9B is a graph showing the quantitation of immunofluorescence of FIG. 9A. Mean γ-H2AX foci per nucleus is plotted. Error bars=SEM. Vector versus shTop2A 668 T test; P value=0.0022. (shTop1 2215 served as an additional control). FIG. 9C shows activated caspase-3 immunofluorescent staining of cytospun Eμ-Myc; Arf^{-/-} lymphoma cells, which reveals an attenuation of doxorubicin-induced apoptosis in shTop2A cells, as compared to vector control cells.

FIG. 10 is a schematic illustration of the in vivo competition assay. Eμ-Myc; Arf^{-/-} lymphoma cells were infected in vitro with an shTop2A or shp53 construct or vector control. Lymphoma cells were then tail-vein injected into syngeneic recipient mice. Upon tumour onset (day 0) mice were treated with DXR (10 mg/kg intra-peritoneal injection) and monitored for overall survival and tumor-free survival time.

FIGS. 11A-B demonstrate that Top2A knockdown caused doxorubicin resistance in vivo. FIG. 11A represents a group of GFP flow cytometry plots showing the results of in vivo competition assay. Lymphoma cells were infected in vitro with GFP-tagged shTop2A 668 or 849, shp53 or vector control constructs (A, left panels). These cells were injected into the tail vein of syngeneic recipient mice (5 mice/cohort) and were monitored daily for tumors by palpation. Upon tumor onset (day 0), one mouse from each cohort was sacrificed and lymphoma cells were assayed for % GFP⁺ (A, middle panels). The remaining mice were treated with doxorubicin (10 mg/kg intra-peritoneal injection), and tumors were harvested upon relapse and assayed for % GFP (A, right panels). FIG. 11B is a graph showing the Kaplan-Meier tumor-free survival curves. Vector, shTop2A and shp53 tumors were FACS-sorted to 100% GFP⁺ prior to injection into recipient mice and DXR-treated as for (A) at day 0.

FIG. 12 is a group of graphs showing that shTOP2A caused doxorubicin resistance in vivo. Eμ-Myc Arf^{-/-} lymphoma cells were infected in vitro with an shTOP2A construct or vector control. In separate experiments, GFP⁺ FACS sorted or unsorted lymphoma cells were tail-vein injected into syngeneic recipient mice. Upon tumor onset (day 0), mice were treated with DXR (10 mg/kg intra-peritoneal injection) and monitored for overall survival and tumor-free survival. shTOP2A-mediated DXR resistance manifested a shorter DXR-induced remission (tumor-free survival) and shorter overall survival as compared to a control (“vector”).

FIGS. 13A-D demonstrate that Top1 knockdown caused camptothecin resistance in vitro and in vivo. FIG. 13A shows that Top1 knockdown caused resistance to camptothecin, but hypersensitivity to the topoisomerase 2 poisons, doxorubicin and etoposide, as shown by a GFP competition assay 24 hours after drug treatment. FIG. 13B summarizes in vitro viability assays of puromycin-selected (shRNA-containing) cells for four independent shRNAs targeting Top1, following 24 hour camptothecin treatment. Error bars are ±SEM from 3 replicates. FIG. 13C shows the results of the immunoblotting assays of Eμ-Myc; Arf^{-/-} lymphoma cell lysates±camptothecin (31 nM CPT, 8 hrs). FIG. 14D shows the Kaplan-Meier survival curve. Eμ-Myc; Arf^{-/-} lymphomas were infected in vitro with vector control or shTop1 2215 and were FACS-sorted to 100% GFP⁺ prior to injection into recipient mice. Upon lymphoma onset (day 0) mice were treated with irinotecan (CPT-11), a clinically relevant camptothecin derivative (50 mg/kg intra-peritoneal injection, daily for 2 days) and monitored for survival.

FIGS. 14A-B demonstrate that multiple Top1 shRNAs caused resistance to camptothecin but sensitivity to the topoisomerase 2 poisons, doxorubicin and etoposide. FIG. 14A summarizes GFP competition assays of Eμ-Myc; Arf^{-/-} lymphomas. Cells were transduced with 4 independent Top1 shRNAs and treated in vitro for 24 hours at the indicated drug doses. FIG. 14B shows that shTop1 caused attenuated camptothecin resistance in Eμ-Myc; p53^{-/-} lymphoma cells, as read by the GFP competition assays 24 hours after treatment.

FIGS. 15 A-C demonstrate that the chemomodification properties of shTop1 knockdown were reproducible across nine out of nine independent Top1 shRNAs. FIG. 15A shows that two of the four Top1 shRNAs described in FIG. 13 (1600, 2215), plus five novel Top1 shRNAs caused sensitivity to the toposiomerase 2 poisons, doxorubicin and etoposide, and caused resistance to the topoisomerase 1 poison, camptothecin, as illustrated by the GFP competition assay in Eμ-Myc; Arf^{-/-} lymphoma cells in vitro. FIG. 15B confirms that Top1 shRNAs knocked down expression of their target, as shown by western blotting. FIG. 15C shows that the suppression of TOP2A or TOP1 in Hela cells mediated resistance to doxorubicin and camptothecin, respectively.

FIGS. 16A-B show that TOP1 suppression sensitized cells to doxorubicin. FIGS. 16A and 16B show that TOP1 knockdown sensitized Eμ-Myc Arf^{-/-} lymphoma cells to the Topoisomerase 2 poisons, doxorubicin and etoposide, as shown by a competition assay (FIG. 16A) and increased in vivo tumor free survival following doxorubicin (10 mg/kg) treatment of transplanted shTOP1 lymphoma cells at tumor onset (day 0), as compared to control (“vector”) (FIG. 16B).

FIGS. 17A-B demonstrate that Top1 knockdown sensitized Eμ-Myc; Arf^{-/-} lymphomas to doxorubicin treatment in vivo. FIG. 17A shows that Top1 knockdown sensitized Eμ-Myc; Arf^{-/-} lymphomas to doxorubicin in vivo, as shown by an increased in vivo tumor free survival following doxorubicin treatment (10 mg/kg, day 0). shTop1 data was pooled from four shTop1 1600 and four shTop1 2215 mice. FIG. 17B demonstrates that predicted changes in topoisomerase expression levels occurred spontaneously during treatment failure in vivo, as shown by immunoblotting analysis of untreated lymphomas and post-doxorubicin treated relapses from FIG. 17A. Lymphomas, sensitized to doxorubicin via shRNA-mediated TOP1 knockdown, displayed TOP1 de-repression (relapse 3) or TOP2A down-regulation (relapses 2 and 4).

FIGS. 18A-B demonstrate that relapsed Top2A-downregulated tumors failing doxorubicin treatment were not broadly drug resistant. FIGS. 18A and 18B summarize the results of treating tumor cells from shTop1 2215 relapsed tumor #4 from FIG. 17 (treated ex-vivo for 24 hours with chemotherapy at the indicated doses). Viability, measured by propidium iodide exclusion flow cytometry, is plotted relative to untreated cells. The Top2A down-regulated relapse displayed doxorubicin resistance (A) but not cross-resistance to cisplatin (B), as compared to a control primary tumor. Together with the observation of striking Top2A downregulation in relapsed tumors, these data suggest that the resistance mechanism is specific to topoisomerase 2 poisons, rather than a general multidrug-resistant pump-based mechanism.

FIG. 19 demonstrates that the combination of Top1 suppression and Top2a inhibition resulted in a G1/S arrest. Tumor cells infected with a control vector or a vector expressing a Top1 shRNA (Top1 shRNA 2215) were treated with 15 ng/ml doxorubicin for 24 hours. Viable cells were stained with propidium iodide for cell cycle analysis. Cells expressing a Top1 shRNA showed an accumulation of cells at the onset of S phase, indicating impaired progression into S phase.

FIGS. 20A-20B show results of a microarray-based method to identify novel genes that are linked to chemotherapeutic drug resistance or sensitization. FIG. 7A is a schematic illustration of the microarray-based shRNA screening. FIG. 7B is a graph showing the principal component analysis of the microarray data. Tight clustering of biological replicate sub-arrays (shown as matching colors) demonstrated data reproducibility. Separation of subarrays under different experimental conditions indicated meaningful, treatment-induced changes in shRNA representation.

FIGS. 21A-21B are tables summarizing the result of statistical analyses, listing shRNAs that caused doxorubicin resistance in Eμ-Myc Arf−/− Lymphoma cells (using Loess normalization and Significance Analysis of Microarrays (SAM)). The most enriched shRNAs are listed, based on a fold change value incorporating values for low and high dose doxorubicin-treated samples relative to untreated samples. shRNAs targeting Top2A were identified from both screenings using the MLS vector (FIG. 8A) and MLP vector (FIG. 8B).

FIGS. 22A-22B are tables summarizing the result of simple data analyses using intensity rankings, listing shRNAs that caused doxorubicin resistance in Eμ-Myc Arf−/− Lymphoma cells. Microarray probes were ranked based on absolute intensity and were compared across experimental conditions. Values indicate the mean rank for untreated samples divided by the mean rank for high dose doxorubicin (day 10). Large positive values signify a rise up the intensity rankings (enrichment) upon doxorubicin treatment. shRNAs targeting Top2A were identified from both screenings using the MLS vector (FIG. 9A) and MLP vector (FIG. 9B).

FIGS. 23A-23B are tables summarizing the result of simple data analyses, listing shRNAs that were most depleted upon doxorubicin treatment of Eμ-Myc Arf−/− Lymphoma cells (using Loess normalisation and Significance Analysis of Microarrays (SAM)). The most depleted shRNAs are listed, based on a fold change value incorporating values for low and high dose doxorubicin-treated samples relative to untreated samples, for both the MLS (FIG. 10A) and MLP (FIG. 10A) screenings.

FIGS. 24A-24B are tables summarizing the result of simple data analyses, listing shRNAs that were most depleted upon doxorubicin treatment of Eμ-Myc Arf−/− Lymphoma cells. Microarray probes were ranked based on absolute intensity and were compared across experimental conditions. Values indicate the mean rank for untreated samples divided by the mean rank for high dose doxorubicin (day 10). Low values signify shRNA depletion upon doxorubicin treatment in the MLS vector (FIG. 11A) and MLP vector (FIG. 11B).

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

The present invention provides methods and compositions useful in identification of genetic alterations that lead to chemotherapeutic agent resistance or sensitization, identification of therapeutic targets for chemotherapy of cancerous cells, identification of cancer patients that may benefit from a particular treatment regimen, and identification of novel chemotherapeutic compounds that enhance the effectiveness of a chemotherapy regimen.

In one aspect, the present invention provides a method to identify a gene whose up-regulation or down-regulation in a cancer cell results in the cancer cell's resistance to a chemotherapeutic agent, comprising: (a) providing a library of RNA interference (RNAi) molecules, wherein each of RNAi molecules inhibits the expression of a target mammalian gene; (b) transfecting a plurality of mammalian cells with the library and expressing the RNAi molecules; (c) treating the transfected cells with the chemotherapeutic agent; and (d) identifying an RNAi molecule that increases the survival of a transfected cell, as compared to cells that do not express the RNAi molecule. AN RNAi molecule that increases the survival of a transfected cell indicates that the down-regulation its target gene results in the cancer cell's resistance to the chemotherapeutic agent. Conversely, an RNAi molecule that reduces the survival of a transfected cell, as compared to cells that do not express the RNAi molecule, indicates that the up-regulation its target gene results in the cancer cell's resistance to the chemotherapeutic agent.

This aspect of the invention provides an effective means to determine whether down-regulation/up-regulation of a gene would lead to resistance to a chemotherapeutic drug. The method of the invention not only can be used to validate cancer therapy targets, but also to identify any candidate gene whose expression or function is responsible for developing resistance/sensitization. These candidate genes may be any relevant genes whose up-regulation/down-regulation confers special advantages to help a tumor cell survive the drug treatment.

The expression or activity of a candidate gene described above may be down-regulated by an antagonist. The antagonist can be any of the antagonists described herein, such as the various RNAi constructs (e.g., shRNA-based or microRNA-based siRNA), antisense polynucleotides, antibodies against the gene products, dominant negative mutants, etc.

RNAi has been widely used to silence or inhibit the expression of a target gene. RNAi is a sequence-specific post-transcriptional gene silencing mechanism triggered by double-stranded RNA (dsRNA). It causes degradation of mRNAs homologous in sequence to the dsRNA. The mediators of the degradation are 21-23-nucleotide small interfering RNAs (siRNAs) generated by cleavage of longer dsRNAs (including hairpin RNAs) by DICER, a ribonuclease III-like protein. Molecules of siRNA typically have 2-3-nucleotide 3′ overhanging ends resembling the RNAse III processing products of long dsRNAs that normally initiate RNAi. When introduced into a cell, they assemble an endonuclease complex (RNA-induced silencing complex), which then guides target mRNA cleavage. As a consequence of degradation of the targeted mRNA, cells with a specific phenotype of the suppression of the corresponding protein product are obtained (e.g., reduction of tumor size, metastasis, angiogenesis, and growth rates).

The small size of siRNAs, compared with traditional antisense molecules, prevents activation of the dsRNA-inducible interferon system present in mammalian cells. This helps avoid the nonspecific phenotypes normally produced by dsRNA larger than 30 base pairs in somatic cells. See, e.g., Elbashir et al., Methods 26:199-213 (2002); McManus and Sharp, Nature Reviews 3:737-747 (2002); Hannon, Nature 418:244-251 (2002); Brummelkamp et al., Science 296:550-553 (2002); Tuschl, Nature Biotechnology 20:446-448 (2002); U.S. Application US2002/0086356 A1; WO 99/32619; WO 01/36646; and WO 01/68836.

In preferred embodiments, the antagonist for the tumor suppressor gene is an siRNA or a precursor molecule thereof, which may be a short hairpin RNA, or a microRNA precursor. A short hairpin RNA (shRNA) is a sequence of RNA that makes a tight hairpin turn (a stem-loop structure) that can be used to silence gene expression. microRNAs (miRNA) are single-stranded RNA molecules of about 21-23 nucleotides; miRNAs are usually processed first from precursor transcripts to short stem-loop structures, then to functional miRNAs. Many microRNA precursors can be used, including without limitation a microRNA comprising a backbone design of miR-15a, -16, -19b, -20, -23a, -27b, -29a, -30b, -30c, -104, -132s, -181, -191, -223. See US 2005-0075492 A1 (incorporated herein by reference).

In certain embodiments, artificial miRNA constructs based on miR-30 (microRNA 30) may be used to express precursor miRNA/shRNA. For example, Silva et al. (Nature Genetics 37: 1281-88, 2005, incorporated herein by reference) have described extensive libraries of pri-miR-30-based retroviral expression vectors that can be used to down-regulate almost all known human (at least 28,000) and mouse (at least 25,000) genes (see RNAi Codex, a single database that curates publicly available RNAi resources, and provides the most complete access to this growing resource, allowing investigators to see not only released clones but also those that are soon to be released, available at http://codex.cshl dot edu). Although such libraries are driven by Pol III promoters, they can be easily converted to the subject Pol II-driven promoters (see Methods in Dickins et al., Nat. Genetics 37: 1289-95, 2005; also see page 1284 in Silva et al., Nat. Genetics 37: 1281-89, 2005).

In certain embodiments, the subject precursor miRNA cassette may be inserted within a gene encoded by the subject vector. For example, the subject precursor miRNA coding sequence may be inserted within an intron, the 5′- or 3′-UTR of a reporter gene, etc. The many possible siRNA precursor molecules (e.g., short hairpin double strand RNA, and the microRNA-based RNA precursors) are described in more details in a section below.

Other methods of RNA interference may also be used in the practice of this invention. See, e.g., Scherer and Rossi, Nature Biotechnology 21:1457-65 (2003) for a review on sequence-specific mRNA knockdown of using antisense oligonucleotides, ribozymes, DNAzymes. See also, International Patent Application PCT/US2003/030901 (Publication No. WO 2004-029219 A2), filed Sep. 29, 2003 and entitled “Cell-based RNA Interference and Related Methods and Compositions.”

Alternatively, the antagonist may be polynucleotides encoding one or more antibodies against the candidate gene product, or a dominant negative mutant of the candidate gene product.

In certain embodiments, a library of RNAi molecules, with each RNAi molecule targeting a particular mammalian gene, are used to down-regulate multiple target genes. In certain embodiments, a genome-wide screening library, containing RNAi constructs representing each open reading frame, are used. In certain embodiments, a relatively small RNAi library, targeting genes of known biological function, are used. In certain embodiments, the RNAi library targets genes that are known to be up-regulated or down-regulated in human cancers. In certain embodiments, the RNAi library may target a mixture of candidate genes of known function and unknown function.

The uncontrolled growth of tumor cells is often caused by mutations in genes that encode for proteins controlling cell division. In addition, multiple mutations may be required to transform a normal cell into a malignant cell. Therefore, the RNAi library may include shRNA molecules targeting a collection of characterized oncogenes and tumor-suppressor genes.

An oncogene is a modified gene, or a set of nucleotides that codes for a protein, that increases the malignancy of a tumor cell. Some oncogenes, usually involved in early stages of cancer development, increase the chance that a normal cell develops into a tumor cell. Commonly seen oncogenes include growth factors or mitogens (such as Platelet-derived growth factor), receptor tyrosine kinases (such as HER2/neu, also known as ErbB-2), cytoplasmic tyrosine kinases (such as the Src-family, Syk-ZAP-70 family and BTK family of tyrosine kinases), regulatory GTPases (such as Ras), cytoplasmic serine/threonine kinases (such as cyclin dependent kinases) and their regulatory subunits, and transcription factors (such as myc).

Any oncogene may be selected as a potential knockdown target using RNAi, including without limitation: ras (e.g., H-ras, N-ras, K-ras, v-ras with various constitutively activating mutations, such as the VI 2 mutation), growth factors (e.g., EGF, PDGF), growth factor receptors (e.g., erbB1-4), signal transducers (e.g., abl, Akt), transcription factors (e.g., myc), apoptosis regulators (e.g., bcl-2), etc.

A tumor suppressor gene is a gene that reduces the probability that a cell in a multicellular organism will turn into a tumor cell. A mutation or deletion of such a gene will increase the probability of the formation of a tumor. The first tumor suppressor protein discovered was the pRb protein in human retinoblastoma; however, recent evidence has also implicated pRb as a tumor survival factor. Another important tumor suppressor is the p53 tumor suppressor protein produced by the TP53 gene.

Any suitable tumor suppressors may be selected as a potential knockdown target using RNAi, including without limitation: p53, BRCA1, BRCA2, APC, p16^{INK4a}, PTEN, NF1, NF2, and RBI.

The RNAi library may also include any genes that that known to exhibit an altered expression level in human cancers. For example, approximately 25-30 percent of breast cancers have an amplification of the HER2/neu gene or overexpression of its protein product. Overexpression of this receptor in breast cancer is associated with increased disease recurrence and worse prognosis. The expression level of a gene can be measured by the gene's mRNA level, protein level, activity level, or other quantity reflected in or derivable from the gene or protein expression data.

Genes of unknown function may also be included in the RNAi library to investigate their potential roles in tumor onset, tumor progression, and drug resistance.

In a preferred embodiment, the “Cancer 1000” shRNA library, containing about 2300 shRNAs targeting about 1000 mouse genes are used. The “Cancer 1000” shRNA library include a mixture of well characterized oncogenes and tumor suppressor genes in addition to many poorly-characterized genes, across many ontological groups, as compiled by literature mining. Similar library design rationale may be easily applied to construct of RNAi libraries targeting genomes of other organisms, such as human.

In certain embodiments, the RNAi targeting a particular candidate gene is transfected into a recipient cell via one or more vectors capable of expressing the shRNA construct. In certain embodiments, the vector is a viral vector. Exemplary viral vector include adenoviral vectors, lentiviral vectors, or retroviral vectors. Many established viral vectors may be used to transfect foreign constructs into cells. The definition section below provides more details regarding the use of such vectors.

Such tranfections may be effected using standard and conventional protocols known in the art. In one embodiment, expression of the RNAi molecule is transient. In another embodiment, the RNAi-expressing construct is stably integrated into the genome of the recipient cell. A single copy of each of RNAi-expressing construct is sufficient for the present invention, but multiple copies integrated at the same or different genomic locations are also within the scope of the invention.

To facilitate the monitoring of the target gene knockdown, and the formation and progression of the cancer, cells harboring the RNAi-expressing construct may additionally comprise a marker construct, such as a fluorescent marker construct. The marker construct expresses a marker, such as green fluorescent protein (GFP), enhanced green fluorescent protein (EGFP), Renilla Reniformis green fluorescent protein, GFPmut2, GFPuv4, yellow fluorescent protein (YFP), enhanced yellow fluorescent protein (EYFP), cyan fluorescent protein (CFP), enhanced cyan fluorescent protein (ECFP), blue fluorescent protein (BFP), enhanced blue fluorescent protein (EBFP), citrine and red fluorescent protein from discosoma (dsRED). Other suitable detectable markers include chloramphenicol acetyltransferase (CAT), luciferase lacZ (β-galactosidase), and alkaline phosphatase. The marker gene may be separately introduced into the cell harboring the shRNA construct (e.g., co-transfected, etc.). Alternatively, the marker gene may be linked to the shRNA construct, and the marker gene expression may be controlled by a separate translation unit under an IRES (internal ribosomal entry site).

In one embodiment, the recipient cell is a mammalian cell. In a preferred embodiment, the recipient cell is a murine cell. In an exemplary embodiment, the recipient cell is a murine Eμ-Myc Arf^{-/-} lymphoma cell (Schmitt et al., 2002). Murine Eμ-Myc Arf^{-/-} lymphoma cells respond reproducibly to low doses of doxorubicin with a robust and rapid apoptotic response within 24 hours (IC50≈7 ng/ml, 16 nM). The deletion of the p19^{Arf} tumor suppressor gene uncouples the proliferative signalling via Myc from the cellular apoptotic response, thus eliminating further selective pressure for lesions in p53 or other components of the DNA damage response, which remain intact during tumorigenesis.

Recipient cells expressing an RNAi construct (e.g., a short hairpin RNA) against a target gene may be further subjected to in vitro assays to determine, for example, cell viability, cell proliferation, apoptosis, cytotoxicity, or target gene expression. The assay results may be compared to a control to determine the effect of the target gene knockdown in the recipient cell. The control may be a parallel sample that has not been treated with the RNAi (e.g., recipient cells transfected with a vector), or which has been treated with an RNAi molecule having a known effect (e.g., a positive effect, a negative effect, or no effect). In other embodiments, the control may be a predetermined value for a particular assay. In an exemplary embodiment, the control is a recipient cell transfected with a vector.

Various methods may be used to determine the growth or viability of recipient cells expressing an RNAi construct in vitro. Such assays may be conducted using commercially available assay kits or methods well known to one or ordinary skill in the art. For example, cell viability can be determined by MTT assay or WST assay. The effect of the target gene knockdown can also be determined using cellular proliferation assays or cellular apoptosis/necrosis assays. In vitro cellular proliferation assays can be performed by determining the amount of cells in a culture over time. Cell numbers may be evaluated using standard techniques. Cellular apoptosis can be measured, for example, using a commercial apoptosis assay kit such as VYBRANT Apoptosis Assay Kit #3 (Molecular Probes). Cells can also be stained with P1 or DAP1 to detect apoptotic nuclei.

In certain embodiments, recipient cells expressing an RNAi construct (e.g., a short hairpin RNA) against a target gene are sorted based on a selectable marker whose expression substantially matches the expression of the RNAi molecule. In one exemplary embodiment, the selectable marker is fluorescence-based. In one exemplary embodiment, the selectable marker is GFP. In one embodiment, cells harboring the selectable marker are sorted using fluorescence-activated cell sorting (FACS). FACS is a powerful system which not only quantifies the fluorescent signal but also separates the cells that contain preselected characteristics (such as fluorescence intensity, size and viability) from a mixed population. Laser light is directed at individual cells as they flow through the FACS. A light scatter pattern is generated when the dense nuclear material of the cell interferes with the path of the laser beam.

In certain embodiments, the effect of a target gene knockdown by RNAi is determined by the percentage of GFP-containing cells. Following chemotherapy treatment, shRNA constructs that cause resistance or sensitization, as compared to control cells, may result in an increase or decrease, respectively, in the percentage of GFP-containing cells in the mixed population.

In certain embodiments, recipient cells surviving the chemotherapy are enriched using FACS sorting. FACS sorting may be performed multiple times to further enrich the surviving cell pool.

Recipient cells expressing an RNAi construct (e.g., a short hairpin RNA) against a target gene may be subsequently transplanted into a recipient non-human animal. Alternatively, after shRNA transfection, the cells may be injected subcutaneously into a recipient non-human animal. The size and growth of tumors in the recipient, tumor-free survival, and overall survival of the recipient may then be observed to investigate the effect of target-gene-knockdown in vivo. The size and growth of tumors may be examined by any of many known method in the art, such as histological methods immunohistochemical methods, TUNEL-staining, etc. In certain embodiments, the non-human animal is a mouse. In certain embodiments, the recipient animal is an immuno-compromised animal, such as a nude mouse.

In certain embodiments, recipient cells harboring an RNAi construct are first enriched by sorting cells expressing a selectable marker, then, the enriched pool of shRNA-containing cells are transplanted or injected into a recipient animal.

In certain embodiments, cells transfected with RNAi constructs are treated with chemotherapeutic agents that target TOP1. A TOP1-targeting agent may be an agent that down-regulates the expression or activity of TOP1 (TOP1 inhibitors). In certain embodiments, the TOP1-targeting agent is an RNAi molecule that down-regulates the expression or activity of TOP1. Alternatively, a TOP1-targeting agent may be a “TOP1 poison,” for example, an agent that stabilizes the cleavable complex consisting of double stranded DNA breaks to which the TOP1 is covalently attached. Commonly used TOP1 poisons include camptothecin, irinotecan and topotecan.

In certain embodiments, cells transfected with RNAi constructs are treated with chemotherapeutic agents that target TOP2, in particular, TOP2A. A TOP2-targeting agent may be an agent that down-regulates the expression or activity of TOP2 (TOP2 inhibitors). In certain embodiments, the TOP2-targeting agent is an RNAi molecule that down-regulates the expression of TOP2, in particular, TOP2A. Alternatively, a TOP2-targeting agent may be a “TOP2 poison,” for example, an agent that stabilizes the cleavable complex consisting of double stranded DNA breaks to which the TOP2 is covalently attached. Examples of TOP2 poisons include doxorubicin (Adriamycin), amsacrine, etoposide, and teniposide, all of which primarily target TOP2A.

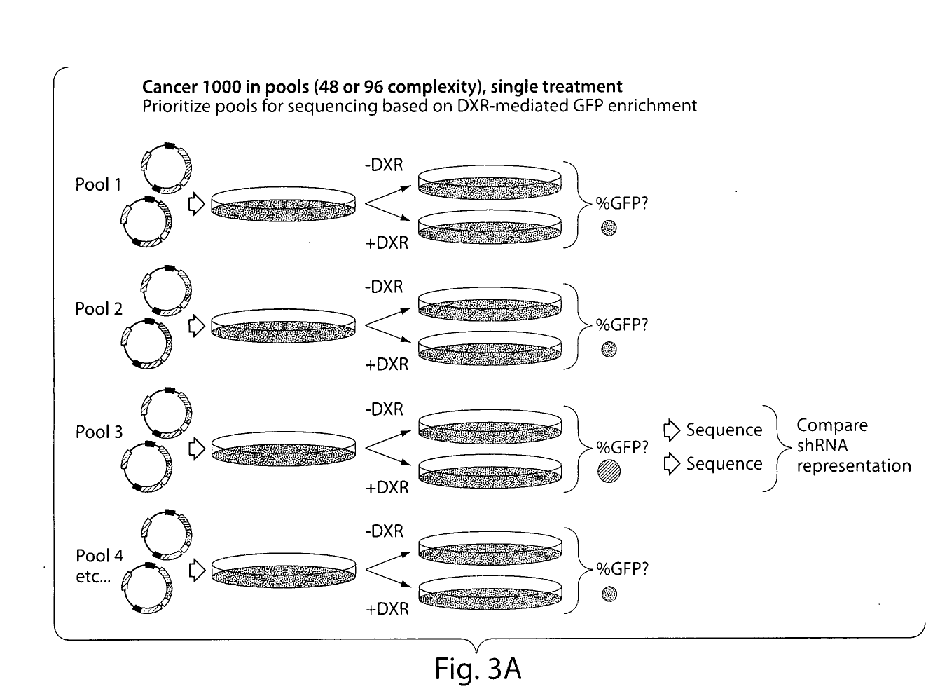

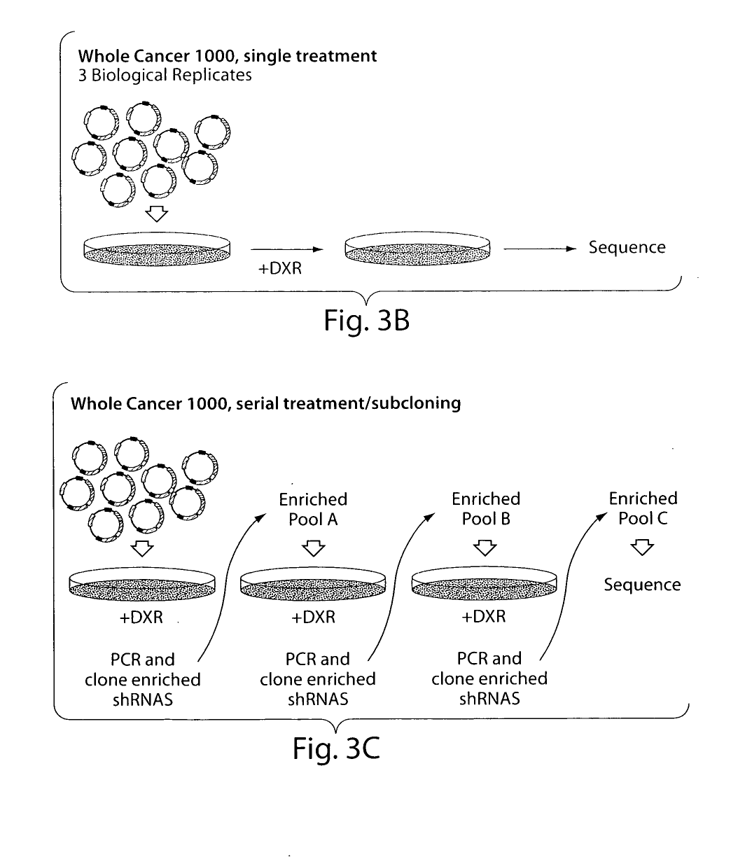

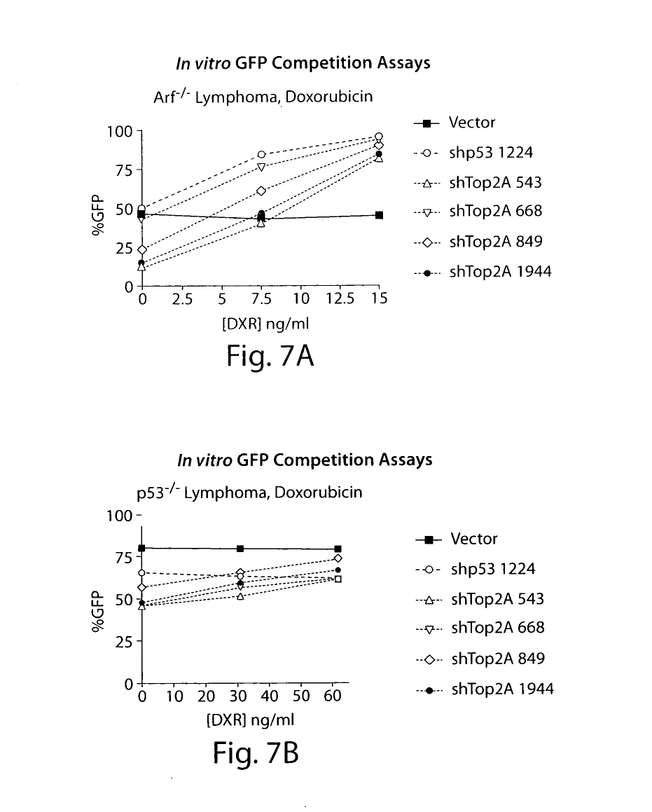

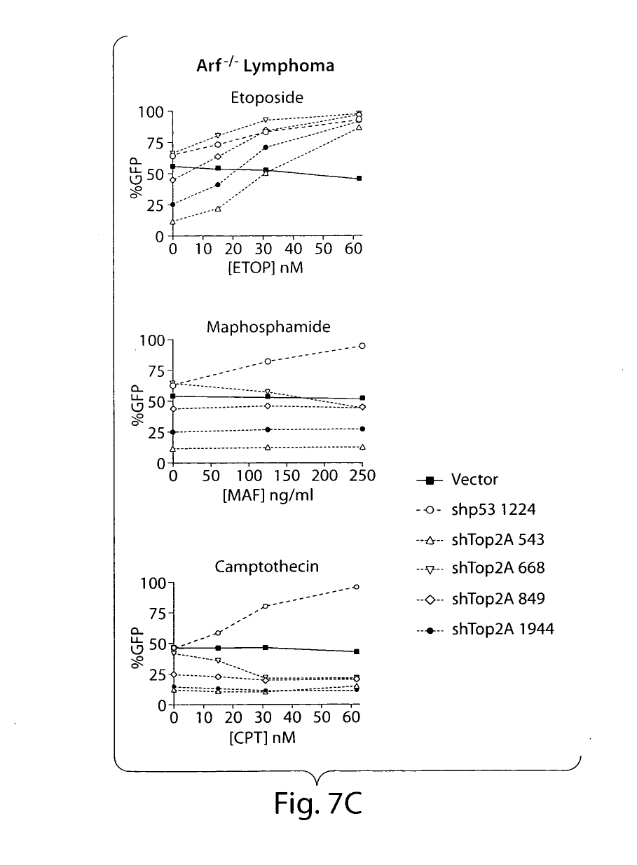

The RNAi-expressing construct that causes sensitization or resistance of a recipient cell to a cancer drug treatment may be isolated and sequenced. Techniques of isolating and sequencing nucleic acid molecules are well known in the art. For example, shRNA sequences may be amplified and sequenced using PCR primers that are unique to the shRNA constructs (an exemplary embodiment is illustrated in Example 3 and FIG. 3B). If desirable, multiple rounds of PCR and cloning of the shRNA molecule may be used between each treatment cycle to enrich the shRNA sequence. Alternatively, the sequence of the RNAi construct that causes sensitization or resistance of a recipient cell to a cancer drug treatment may be identified using the microarray technology that is well known in the art (an exemplary embodiment is illustrated in Example 8 and FIG. 7A; see below for a brief description of the microarray technology). For example, an RNAi molecule that causes a recipient cell resistant to a cancer drug treatment will likely be highly enriched upon drug treatment; therefore, the fluorescence intensity will likely increase as the drug dosage increases. Conversely, an RNAi molecule that causes a recipient cell sensitive to a cancer drug treatment will likely be depleted upon drug treatment; therefore, the fluorescence intensity will likely decrease as the drug dosage increases. By comparing fluorescence intensities of samples from parallel treatments with different dosages, one can identify a gene whose down regulation causes resistance or sensitization of a cancer cell.

The methods disclosed in the present invention may be used to design a rational therapy, or select patient populations for the purposes of clinical trials. The method may be used to identify one or more genes that are linked to drug-resistance of any known chemotherapeutic drugs. The expression profile of those genes that are linked to cancer drug resistance may be compiled, and the data may be used for patient stratification and individual patient profiling. For example, a metric of correlation between the expression level of a particular gene (e.g., TOP2) and the cancer cell's resistance to a cancer drug (e.g., Doxorubicin) may be prepared. A cancer patient will first be screened to determine the expression level of those genes that are linked to drug resistance. The expression profile of those genes in the patient is then compared to the pre-compiled correlation data, or a control, and the output would indicate whether the patients will likely respond to a particular cancer drug.

In another aspect, the present invention provides a method to identify a gene whose up-regulation or down-regulation in a cancer cell results in the cancer cell's sensitivity to a chemotherapeutic agent, comprising: (a) providing an RNA interference (RNAi) molecule that inhibits the expression of a candidate gene; (b) transfecting a plurality of mammalian cells with the RNAi molecule; (c) treating the transfected cells with the chemotherapeutic agent; (d) monitoring survival of the transfected cells and control cells that do not express the RNAi molecule. AN RNAi molecule that reduces the survival of a transfected cell, as compared to cells that do not express the RNAi molecule, indicates that the down-regulation of its target gene enhances the cancer cell's sensitivity to the chemotherapeutic agent. Conversely, RNAi molecule that enhances the survival of a transfected cell indicates that the up-regulation of its target gene may result in the cancer cell's sensitivity to the chemotherapeutic agent.

In certain embodiments, the chemotherapeutic agent targets TOP1. In some embodiments, the chemotherapeutic agent is a TOP1 inhibitor. In certain embodiments, the chemotherapeutic agent is a TOP1 poison. In certain embodiments, the chemotherapeutic agent targets TOP2, in particular, TOP2A. In certain embodiments, the chemotherapeutic agent is a TOP2 inhibitor. In certain embodiments, the chemotherapeutic agent is a TOP2 poison.

In one aspect, the present invention provides a method to identify a gene whose up-regulation or down-regulation in a cancer cell results in the cancer cell's sensitivity to a chemotherapeutic agent, comprising: (a) providing a library of RNA interference (RNAi) molecules, wherein each of RNAi molecules inhibits the expression of a target mammalian gene; (b) transfecting a plurality of mammalian cells with the library and expressing the RNAi molecules; (c) treating the transfected cells with the chemotherapeutic agent; and (d) identifying an RNAi molecule that decreases the survival of a transfected cell, as compared to cells that do not express the RNAi molecule. AN RNAi molecule that decreases the survival of a transfected cell indicates that the down-regulation its target gene results in the cancer cell's sensitivity to the chemotherapeutic agent. Conversely, an RNAi molecule that increases the survival of a transfected cell, as compared to cells that do not express the RNAi molecule, indicates that the up-regulation its target gene results in the cancer cell's sensitivity to the chemotherapeutic agent.

In certain embodiments, the chemotherapeutic agent targets TOP1. In some embodiments, the chemotherapeutic agent is a TOP1 inhibitor. In certain embodiments, the chemotherapeutic agent is a TOP1 poison. In certain embodiments, the chemotherapeutic agent targets TOP2, in particular, TOP2A. In certain embodiments, the chemotherapeutic agent is a TOP2 inhibitor. In certain embodiments, the chemotherapeutic agent is a TOP2 poison. In certain embodiment, the target gene is identified using microarray.

In another aspect, the present invention provides a method for identifying a cancer patient who may benefit from a treatment with a chemotherapeutic agent, comprising (a) obtaining a cancer cell from the patient, and (b) determining the expression level of a gene that whose altered expression level lead to resistance or sensitization of a cancer cell to the chemotherapeutic agent. The RNAi-based method described above can be used to quickly and efficiently identify those genes whose altered expression level lead to resistance or sensitization of a cancer cell to the chemotherapeutic agent. Once the sequences of the genes are known, the expression level of these genes from a cancer patient can then be compared to a control; a difference in the expression level between the cancer cell and the control may predict how a patient will respond to a treatment with the chemotherapeutic agent.

One of the recurring problems of cancer therapy is that a patient in remission (after the initial treatment by surgery, chemotherapy, radiotherapy, or combination thereof) may experience relapse. The recurring cancer in those patients is frequently resistant to the apparently successful initial treatment. In fact, certain cancers in patients initially diagnosed with the disease may be already resistant to conventional cancer therapy even without first being exposed to such treatment. Chemotherapy can be physically exhausting for the patient. Virtually all chemotherapeutic regimens can cause depression of the immune system. Other common side-effects include hair loss, nausea and vomiting, diarrhea or constipation, and hemorrhage. Thus there is a need to determine whether a cancer patient may benefit from a chemotherapeutic treatment prior to the commencement of the treatment.

In one embodiment, a cancer patient is screened based on the expression level of TOP2 in a cancer cell sample. An increased expression of TOP2, as compared to a control, indicates that the patient may benefit from a treatment with a TOP2-targeting chemotherapeutic agent. Conversely, a decreased expression of TOP2, as compared to a control, indicates that the patient may be resistant to a treatment with a TOP2-targeting chemotherapeutic agent.

In another embodiment, a cancer patient is screened based on the expression level of TOP1 in a cancer cell sample. An increased expression of TOP1, as compared to a control, indicates that the patient may benefit from a treatment with a TOP1-targeting chemotherapeutic agent. Conversely, a decreased expression of TOP1, as compared to a control, indicates that the patient may be resistant to a treatment with a TOP1-targeting chemotherapeutic agent.

In another embodiment, a cancer patient is screened based on the expression level of TOP1 in a cancer cell sample. A decreased expression of TOP1, as compared to a control, indicates that the patient may be highly sensitive to a treatment with a TOP2-targeting chemotherapeutic agent. Conversely, an increased expression of TOP1, as compared to a control, indicates that the patient may be resistant to a treatment with a TOP2-targeting chemotherapeutic agent. In certain embodiments, the TOP2-targeting chemotherapeutic agent is a TOP2 poison.

In another embodiments, a cancer patient is screened based on the expression level of Skp2 in a cancer cell sample. A decreased expression of Skp2, as compared to a control, indicates that the patient may be resistant to a treatment with a TOP2-targeting chemotherapeutic agent (such as doxorubicin) or a TOP1-targeting chemotherapeutic agent (such as camptothecin). Conversely, an increased expression of Skp2, as compared to a control, indicates that the patient may be sensitive to a treatment with a TOP2-targeting chemotherapeutic agent (such as doxorubicin) or TOP1-targeting chemotherapeutic agent (such as camptothecin).

In another embodiment, a cancer patient is screened based on the expression level of Bmi1 in a cancer cell sample. A decreased expression of Bmi1, as compared to a control, indicates that the patient may be highly sensitive to a treatment with a TOP2-targeting chemotherapeutic agent. Conversely, an increased expression of Bmi1, as compared to a control, indicates that the patient may be resistant to a treatment with a TOP2-targeting chemotherapeutic agent. In certain embodiments, the TOP2-targeting chemotherapeutic agent is a TOP2 poison.

The expression level of TOP1, TOP2, or other genes that are linked to cancer drug sensitization or resistance, be measured by mRNA level, protein level, activity level, or other quantity reflected in or derivable from the gene or protein expression data. For example, the mRNA level of TOP1 or TOP2 may be measured using microarray technology that is well known in the art. Briefly, in a typical microarray experiment, a microarray is hybridized with differentially labeled RNA or DNA populations derived from two different samples. Most commonly, RNA (either total RNA or mRNA) is isolated from cells or tissues of interest and is reverse transcribed to yield cDNA. Labeling is usually performed during reverse transcription by incorporating a labeled nucleotide in the reaction mixture. Although various labels can be used, most commonly the nucleotide is conjugated with the fluorescent dyes Cy3 or Cy5. For example, Cy5-dUTP and Cy3-dUTP can be used. cDNA derived from one sample is labeled with one fluor while cDNA derived from a second sample is labeled with the second fluor. Similar amounts of labeled material from the two samples are cohybridized to the microarray. In the case of a microarray experiment in which the samples are labeled with Cy5 (which fluoresces red) and Cy3 (which fluoresces green), the primary data (obtained by scanning the microarray using a detector capable of quantitatively detecting fluorescence intensity) are ratios of fluorescence intensity (red/green, R/G). These ratios represent the relative concentrations of cDNA molecules that hybridized to the cDNAs represented on the microarray and thus reflect the relative expression levels of the mRNA corresponding to each cDNA/gene represented on the microarray.

Alternatively, the mRNA level TOP1 or TOP2, or other genes that are linked to cancer drug sensitization or resistance, can be measured by polymerase chain reaction (PCR), a technique well known in the art. Briefly, one or more sets of oligonucleotide primers are annealed to a target sequence of interest, and the annealed primers are extended simultaneously to generate double-stranded (ds) copies of the target sequence. The primers are extended by a thermal-stable polymerase (McPherson, M. Ed. (1995) PCR 2: A Practical Approach, IRL Press at Oxford University Press, Oxford). The primers may be about 5-50 nucleotides in length. Real-time polymerase chain reaction, also called quantitative real time PCR (QRT-PCR) or kinetic polymerase chain reaction, may be highly useful to determine the expression level of a target gene because the technique can simultaneously quantify and amplify a specific part of a given polynucleotide. The QRT-PCR procedure follows the general pattern of polymerase chain reaction, but the DNA is quantified after each round of amplification. Two common methods of quantification are the use of fluorescent dyes that intercalate with double-strand DNA, and modified DNA oligonucleotide probes that fluoresce when hybridized with a complementary DNA. QRT-PCR can be combined with reverse transcription polymerase chain reaction to quantify low abundance messenger RNA (mRNA), enabling one to quantify relative gene expression at a particular time, or in a particular cell or tissue type.

The expression level of TOP1 or TOP2, or other genes that are linked to cancer drug sensitization or resistance, may also be measured by protein level using any art-known method. Traditional methodologies for protein quantification include 2-D gel electrophoresis, mass spectrometry and antibody binding. Preferred methods for assaying target protein levels in a biological sample include antibody-based techniques, such as immunoblotting (western blotting), immunohistological assay, enzyme linked immunosorbent assay (ELISA), radioimmunoassay (RIA), or protein chips. Gel electrophoresis, immunoprecipitation and mass spectrometry may be carried out using standard techniques, for example, such as those described in Molecular Cloning A Laboratory Manual, 2^{nd} Ed., ed. by Sambrook, Fritsch and Maniatis (Cold Spring Harbor Laboratory Press: 1989), Harlow and Lane, Antibodies: A Laboratory Manual (1988 Cold Spring Harbor Laboratory), G. Suizdak, Mass Spectrometry for Biotechnology (Academic Press 1996), as well as other references cited herein.

The expression level of TOP1 or TOP2, or other genes that are linked to cancer drug sensitization or resistance, can also be measured by the activity level of the gene product using any art-known method.

In certain embodiments, it may be useful to compare the expression level of TOP1 or TOP2, or other genes that are linked to cancer drug sensitization or resistance, to a control. The control may be a measure of the expression level of TOP1 or TOP2, or other genes that are linked to cancer drug sensitization or resistance, in a quantitative form (e.g., a number, ratio, percentage, graph, etc.) or a qualitative form (e.g., band intensity on a gel or blot, etc.). A variety of controls may be used. Levels of TOP1 or TOP2 (or other genes that are linked to cancer drug sensitization or resistance) expression from a healthy individual may also be used as a control. Alternatively, the control may be expression levels of TOP1 or TOP2 (or other genes that are linked to cancer drug sensitization or resistance) from the individual being treated at a time prior to treatment or at a time period earlier during the course of treatment. Still other controls may include expression levels present in a database (e.g., a table, electronic database, spreadsheet, etc.).

In certain embodiments, the cancer patient may be suffering from or susceptible to: cancer of the bladder, breast, colon, kidney, liver, lung (including small cell lung cancer), esophagus, gall bladder, ovary, pancreas, stomach, cervix, thyroid, prostate, and skin, including (squamous cell carcinoma); leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; acute and chronic myelogenous leukemia, myelodysplastic syndrome and promyelocytic leukemia; fibrosarcoma, rhabdomyosarcoma; astrocytoma, neuroblastoma, glioma and schwannomas; melanoma, seminoma, teratocarcinoma, osteosarcoma, xenoderoma pigmentosum, keratoctanthoma, thyroid follicular cancer and Kaposi's sarcoma. In an exemplary embodiment, the patient is suffering from or susceptible to acute myelogenous leukemia.

The present invention further discloses methods of treating cancer patients who will likely benefit from a treatment with TOP2-targeting chemotherapeutic agent (identified using the methods described above), comprising administering to the patients a TOP2-targeting chemotherapeutic agent.

In another aspect, the present invention discloses a method for identifying an agent that enhances the effectiveness of a treatment with a TOP2-trageting chemotherapeutic agent. Using the RNAi-based screening method disclosed above, it was discovered that knocking down the expression of TOP1 or Bmi1 can sensitize a cell to a TOP2-targeting chemotherapeutic agent. Therefore, the disclosed method may be used to screen for potential TOP1-inhibiting agents or Bmi1-inhibiting agents. After contacting a mammalian cell with the candidate agent, a decreased expression level of TOP1 in the presence of the agent, as compared to control, may indicate that the candidate agent is a TOP1 inhibitor and may be used in conjunction with a TOP2-targeting cancer drug to enhance to effectiveness of the TOP2-targeting drug. In certain embodiments, the TOP2-targeting therapeutic agent is a TOP2 poison. In certain embodiments, the TOP2 poison is a TOP2A poison. Similarly, after contacting a mammalian cell with the candidate agent, a decreased expression level of Bmi1 in the presence of the agent, as compared to control, may indicate that the candidate agent is a Bmi1 inhibitor and may be used in conjunction with a TOP2-targeting cancer drug to enhance to effectiveness of the TOP2-targeting drug. In certain embodiments, the TOP2-targeting therapeutic agent is a TOP2 poison. In certain embodiments, the TOP2 poison is a TOP2A poison.

The candidate agent may be a chemical compound, a mixture of chemical compounds, a biological macromolecule (such as a nucleic acid, an antibody, an antibody fragment, a protein, a protein fragment, or a peptide), or an extract made from biological materials such as bacteria, plants, fungi, or animal cells or tissues. Suitable nucleic acid compounds include, for example, aptamers, enzymatic nucleic acids (e.g., ribozymes or DNAzymes), antisense nucleic acids, and siRNAs.

Commonly used TOP2-targeting drugs include doxorubicin, etoposide, mitoxantrone, mAMSA, amonafide, batracylin and menadione. The TOP2-targeting drugs and the TOP1 inhibiting agent may be administered serially or simultaneously to a cancer patient.

In another aspect, the present invention discloses a method of treating a cancer patient, comprising administering to the patient a TOP1 inhibitor and a TOP2-targeting chemotherapeutic agent. A TOP1 inhibitor is an agent that down-regulates the expression or activity of TOP1. A TOP1 inhibitor normally does not covalently crosslink or stabilize the TOP1-DNA complex. Commonly used TOP2-targeting drugs include doxorubicin, etoposide, mitoxantrone, mAMSA, amonafide, batracylin and menadione. The TOP2-targeting drugs and the TOP1 inhibitor may be administered serially or simultaneously to a cancer patient.

In certain embodiments, the TOP1 inhibitor is an RNAi molecule (such as shRNA or miRNA) that inhibits the expression of TOP1. In certain embodiments, the RNAi molecule is part of a viral vector. Commonly used viral vectors include adenoviral vectors, a lentiviral vectors, or a retroviral vectors.

In another aspect, the present invention discloses a method of treating a cancer patient, comprising administering to the patient a Bmi1 inhibitor and a TOP2-targeting chemotherapeutic agent. A Bmi1 inhibitor is an agent that down-regulates the expression or activity of TOP1. Commonly used TOP2-targeting drugs include doxorubicin, etoposide, mitoxantrone, mAMSA, amonafide, batracylin and menadione. The TOP2-targeting drugs and the Bmi1 inhibitor may be administered serially or simultaneously to a cancer patient.

In certain embodiments, the Bmi1 inhibitor is an RNAi molecule (such as shRNA or miRNA) that inhibits the expression of Bmi1. In certain embodiments, the RNAi molecule is part of a viral vector. Commonly used viral vectors include adenoviral vectors, a lentiviral vectors, or a retroviral vectors.

In another aspect, the present invention discloses a method for inhibiting a cancer cell growth, comprising contacting the cancer cell with a TOP1 inhibitor and a TOP2-targeting chemotherapeutic agent. A TOP1 inhibitor is an agent that down-regulates the expression or activity of TOP1. A TOP1 inhibitor normally does not covalently crosslink or stabilize the TOP1-DNA complex. Commonly used TOP2-targeting drugs include doxorubicin, etoposide, mitoxantrone, mAMSA, amonafide, batracylin and menadione. The TOP2-targeting drugs and the TOP1 inhibitor may be administered serially or simultaneously to a cancer patient.

In certain embodiments, the TOP1 inhibitor is an RNAi molecule (such as shRNA or miRNA) that inhibits the expression of TOP1. In certain embodiments, the RNAi molecule is part of a viral vector. Commonly used viral vectors include adenoviral vectors, a lentiviral vectors, or a retroviral vectors.

In another aspect, the present invention discloses a method for inhibiting a cancer cell growth, comprising contacting the cancer cell with a Bmi1 inhibitor and a TOP2-targeting chemotherapeutic agent. A Bmi1 inhibitor is an agent that down-regulates the expression or activity of TOP1. Commonly used TOP2-targeting drugs include doxorubicin, etoposide, mitoxantrone, mAMSA, amonafide, batracylin and menadione. The TOP2-targeting drugs and the Bmi1 inhibitor may be administered serially or simultaneously to a cancer patient.

In certain embodiments, the Bmi1 inhibitor is an RNAi molecule (such as shRNA or miRNA) that inhibits the expression of Bmi1. In certain embodiments, the RNAi molecule is part of a viral vector. Commonly used viral vectors include adenoviral vectors, a lentiviral vectors, or a retroviral vectors.

In another aspect, the invention provides pharmaceutical preparations comprising the RNAi constructs disclosed herein. A pharmaceutical preparation may further comprise a polypeptide, such as a polypeptide selected from amongst serum polypeptides, cell targeting polypeptides and internalizing polypeptides. Examples of cell targeting polypeptides include a polypeptide comprising a plurality of galactose moieties for targeting to hepatocytes (e.g., asialoglycoproteins, such as asialofetuin), a transferrin polypeptide for targeting to neoplastic cells and an antibody that binds selectively to a cell of interest. A polypeptide may be associated with the RNAi constructs, covalently or non-covalently.

In certain embodiments, a pharmaceutical preparation for delivery to a subject may comprise an RNAi construct of the invention and a pharmaceutically acceptable carrier. Optionally, the pharmaceutically acceptable carrier is selected from pharmaceutically acceptable salts, ester, and salts of such esters. A pharmaceutical preparation may be packaged with instructions for use with a human or other animal patient. In certain embodiment, the RNAi construct of the invention is administered systemically or locally as part of a pharmaceutical composition. In certain embodiments, the RNAi construct of the invention is administered simultaneously or serially with another chemotherapeutic agent, such as TOP2A poisons.

### DEFINITIONS

As used herein, the terms “cancer” or “tumor” are used interchangeably.

Throughout this specification and embodiments, the word “comprise” or variations such as “comprises” or “comprising” will be understood to imply the inclusion of a stated integer or group of integers but not the exclusion of any other integer or group of integers.

The term “chemotherapeutic agent” includes any molecule useful in the treatment of cancer. Such molecules include, without limitation, polypeptides (including proteins), such as antibodies, peptides, organic and inorganic small molecules, and nucleic acid (DNA and RNA) molecules. A chemotherapeutic agent may be a substance that inhibits or prevents the function of cancer cells, inhibits the proliferation or viability of cancer cells, causes destruction (e.g., senescence and apoptosis) of cancer cells, stimulates the cancer cells' clearance by the immune system, causes transient cell cycle arrest, causes mitotic catastrophe, or causes autophagocytosis.

The term “down-regulation” refers to decreased expression or activity of a gene product. Down-regulation of a gene product could be caused, for example, by null mutations, loss of alleles, inactivating mutation (e.g., dominant negative mutations), gene copy number variations (e.g., reduced in gene copy number), and otherwise reduced expression or activity. The term “up-regulation” refers to increased expression or activity of a gene product. Up-regulation of a gene product could be caused, for example, by gene copy number variations (e.g., gene amplification), loss of regulation, increased protein stability, activating mutation (e.g., constitutively active kinases), chromosome translocation, and otherwise increased expression or activity.

A “short hairpin RNA (shRNA)” refers to a segment of RNA that is complementary to a portion of a target gene (e.g., complementary to one or more transcripts of a target gene), and has a stem-loop (hairpin) structure that can be used to silence gene expression. When a nucleic acid construct encoding a short hairpin RNA is introduced into a cell, the cell incurs partial or complete loss of expression of the target gene. In this way, a short hairpin RNA functions as a sequence-specific expression inhibitor or modulator in transfected cells. The use of short hairpin RNAs facilitates the down-regulation of the target gene and allows for analysis of hypomorphic alleles. Short hairpin RNAs useful in the invention can be produced using a wide variety of well known RNA interference (“RNAi”) techniques. The invention may be practiced using short hairpin RNAs that are synthetically produced as well as microRNA (miRNA) molecules that are found in nature and can be remodeled to function as synthetic silencing short hairpin RNAs. DNA vectors that express perfect complementary short hairpins RNAs (shRNAs) are commonly used to generate functional siRNAs.

MicroRNAs (miRNAs) are endogenously encoded ˜22-nt-long RNAs that are generally expressed in a highly tissue- or developmental-stage-specific fashion and that post-transcriptionally regulate target genes. More than 200 distinct miRNAs having been identified in plants and animals, these small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Importantly, miRNAs are expressed in a highly tissue-specific or developmentally regulated manner and this regulation is likely key to their predicted roles in eukaryotic development and differentiation. Analysis of the normal role of miRNAs will be facilitated by techniques that allow the regulated over-expression or inappropriate expression of authentic miRNAs in vivo, whereas the ability to regulate the expression of siRNAs will greatly increase their utility both in cultured cells and in vivo. Thus one can design and express artificial microRNAs based on the features of existing microRNA genes, such as the gene encoding the human miR-30 microRNA. These miR30-based shRNAs have complex folds, and, compared with simpler stem/loop style shRNAs, are more potent at inhibiting gene expression in transient assays.

miRNAs are first transcribed as part of a long, largely single-stranded primary transcript (Lee et al., EMBO J. 21: 4663-4670, 2002). This primary miRNA transcript is generally, and possibly invariably, synthesized by RNA polymerase II (pol II) and therefore is normally polyadenylated and may be spliced. It contains an ˜80-nt hairpin structure that encodes the mature ˜22-nt miRNA as part of one arm of the stem. In animal cells, this primary transcript is cleaved by a nuclear RNaseIII-type enzyme called Drosha (Lee et al., Nature 425: 415-419, 2003) to liberate a hairpin miRNA precursor, or pre-miRNA, of ˜65 nt, which is then exported to the cytoplasm by exportin-5 and the GTP-bound form of the Ran cofactor (Yi et al., Genes Dev. 17: 3011-3016, 2003). Once in the cytoplasm, the pre-miRNA is further processed by Dicer, another RNaseIII enzyme, to produce a duplex of ˜22 bp that is structurally identical to an siRNA duplex (Hutvagner et al., Science 293: 834838, 2001). The binding of protein components of the RNA-induced silencing complex (RISC), or RISC cofactors, to the duplex results in incorporation of the mature, single-stranded miRNA into a RISC or RISC-like protein complex, whereas the other strand of the duplex is degraded (Bartel, Cell 116: 281-297, 2004).

The miR-30 architecture can be used to express miRNAs or siRNAs from pol II promoter-based expression plasmids. See also Zeng et al., Methods in Enzymology 392: 371-380, 2005 (incorporated herein by reference).

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof.

In certain embodiments, useful interfering RNAs can be designed with a number of software programs, e.g., the OligoEngine siRNA design tool available at www.oligoengine.com. The siRNAs of this invention may be about, e.g., 19-29 base pairs in length for the double-stranded portion. In some embodiments, the siRNAs are short hairpin RNAs having an about 19-29 bp stem and an about 4-34 nucleotide loop. Preferred siRNAs are highly specific for a region of the target gene and may comprise any about 19-29 bp fragment of the mRNA of a target gene, with at least one, preferably at least two or three, bp mismatch with a nontarget gene-related sequence. In some embodiments, the preferred siRNAs do not bind to RNAs having more than 3 mismatches with the targeting region.

The term “vector” refers to a nucleic acid molecule capable of transporting another nucleic acid to which it has been linked. Vectors include, but are not limited to, plasmids, phagemids, viruses, other vehicles derived from viral or bacterial sources that have been manipulated by the insertion or incorporation of the nucleic acid sequences for producing the precursor shRNA, and free nucleic acid fragments which can be attached to these nucleic acid sequences. One type of vector is a plasmid, which refers to a circular double stranded DNA loop into which additional DNA segments may be ligated. A preferred type of vector for use in this application is a viral vector, wherein additional DNA segments may be ligated into a viral genome that is usually modified to delete one or more viral genes. Certain vectors are capable of autonomous replication in a host cell into which they are introduced (e.g., vectors having an origin of replication which functions in the host cell). Other vectors can be integrated stably into the genome of a host cell upon introduction into the host cell, and are thereby replicated along with the host genome.

The invention also encompasses host cells transfected with the subject vectors, including host cell that transiently express he transfected shRNA or microRNA constructs, and cells lines with stably integrated the shRNA or microRNA constructs. In certain embodiments, the subject host cell contains one or more copies of the construct expressing the desired shRNA or microRNA.

The invention also encompasses animals comprising host cells transfected with the subject vectors.

### EXEMPLIFICATIONS

The invention now being generally described, it will be more readily understood by reference to the following examples, which are included merely for purposes of illustration of certain aspects and embodiments of the present invention, and are not intended to limit the invention.

### Example 1

### Selecting an RNAi Library

RNA interference (RNAi) exploits a mechanism of gene regulation whereby double stranded RNAs are processed by a conserved cellular machinery to suppress the expression of genes containing homologous sequences. Importantly, libraries of DNA-based vectors encoding short hairpin RNAs (shRNAs) capable of targeting most genes in the human and mouse genome have been produced and enable forward genetic screens to be performed in mammalian cells. Indeed, using human tumor-derived cell lines treated in vitro, RNAi has been used to evaluate potential drug targets, or to investigate mechanisms of drug action and drug resistance by screening for new molecules that modulate the response of tumor derived cell lines to a given chemotherapeutic agent.

As in vivo studies of drug sensitivity and resistance require stable gene knockdown, we performed our initial in vitro screens using retrovirally-encoded shRNAs based on the MiR-30 microRNA. Importantly, these shRNAs can stably and efficiently knockdown target genes when expressed at single copy in the genome.

To identify a gene whose inactivation in a cancer cell results in the cancer cell's resistance to an apoptotic-inducing cancer drug, it is important to choose a suitable RNAi library. A genome-wide screening library, with shRNA constructs representing each open reading frame, may be used. Alternatively, one may choose a very small RNAi library of known biological function.

The screening was performed using the “Cancer 1000” shRNA subset containing about 2300 shRNAs targeting about 1000 mouse genes (2-3 shRNAs per gene). The “Cancer 1000” shRNA library include a mixture of well characterized oncogenes and tumor suppressor genes in addition to many poorly-characterized genes, across many ontological groups, as compiled by literature mining (Harvard Institute of Proteomics). This library represented a balance between the relatively narrow biology of small, functional gene sets and a genome-wide screening.

In this particular example, the RNAi library of choice was the Hannon-Elledge shRNA library (Silva et al., 2005), administered to lymphoma cells via retroviral infection. The stable integration and knockdown via retroviral constructs, even at single copy (Dickins et al., 2005), allows for longer term experiments and easier shRNA construct recovery than transfection-based techniques.

### Example 2

### Subcloning the “Cancer 1000” Library into Recipient Vectors

To improve gene knockdown and facilitate in vivo experiments, all of the existing murine shRNAs targeting the cancer 1000 set (˜2300 shRNAs, 2-3 shRNAs per gene) were cloned into an MSCV-based vector that co-expressed green fluorescent protein. Briefly, a MiR-30-based shRNA library targeting the cancer 1000 gene set was subcloned into LMP and LMS (MSCV-based vectors) in pools of 96 or 48 shRNAs, respectively. Targeting sequences were selected based on RNAi Codex algorithms or BIOPREDsi design.

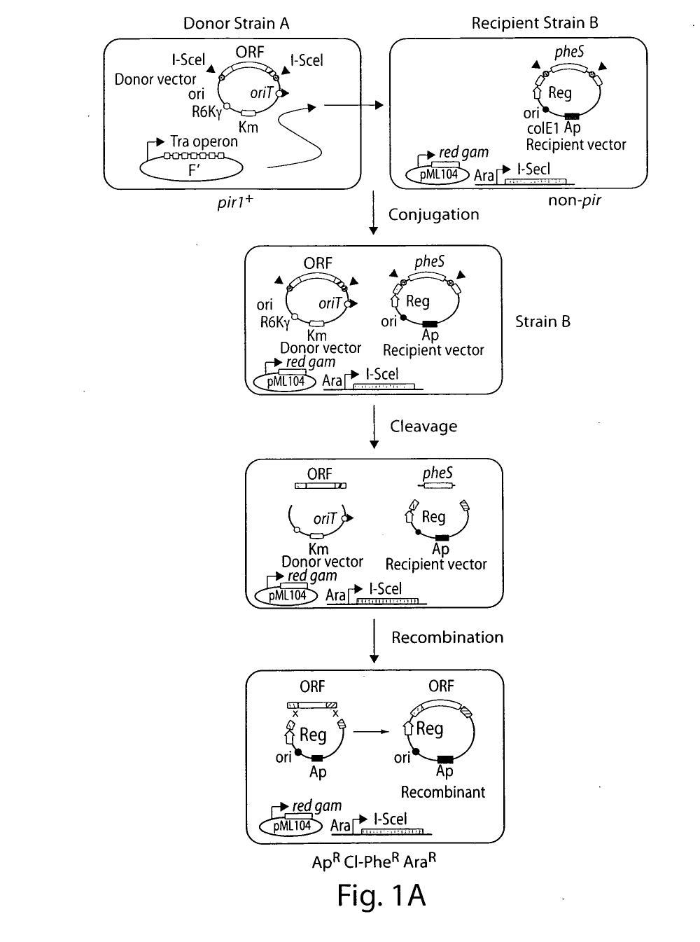

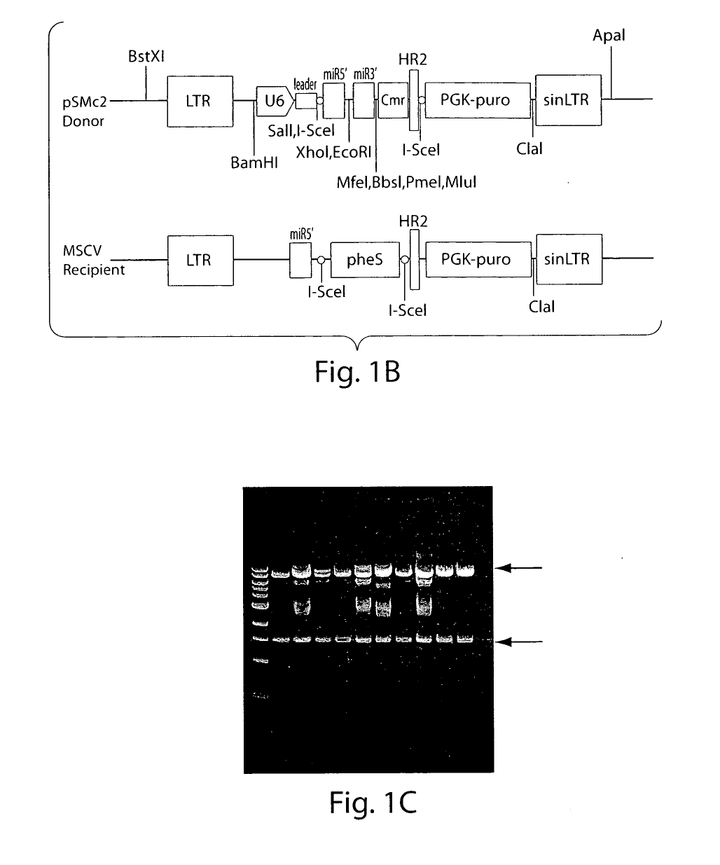

Two methods were used to subclone the “Cancer 1000” library into recipient vectors. The first method was bacterial mating, using “Mating-Assisted Genetically Integrated Cloning” (MAGIC) (Li and Elledge, 2005). The MAGIC system consists of a donor vector (the library vector), in which the fragment of interest is flanked by two different homology regions, H1 and H2, which in turn are flanked with linked I-SceI sites. The donor vector also includes an F′ origin and a conditional origin of replication (RK6^{γ}). The recipient vector, which also contains I-SceI-linked H1 and H2 sites surrounding a negative selectable marker (pheS), resides in a bacterial strain that contains an inducible I-SceI gene. After transfer of the donor vector into the recipient host by bacterial mating, I-SceI cleaves both donor and recipient vectors, and these breaks are healed by homologous recombination via the H1 and H2 sequences. Selection against the unrecombined recipient containing pheS and I-SceI sites and for the capture of the appropriate insert (chloramphenicol resistance) gives essentially 100% recovery of the desired plasmid (FIG. 1A).

Mating cassettes were incorporated into the pSM2 vector, allowing an intracellular, recombination method for shRNA transfer to a recipient vector. Sugars were used to induce the mating and antibiotics were used to select the correctly recombined products. We tested the system by subcloning a subset of the cancer 1000 library, one-by-one in a multiwell format, into an MSCV-derived vector, modified to contain the appropriate sequences for excision, recombination and negative selection (FIG. 1B). The resultant DNA clones were tested by restriction digest (FIG. 1C).

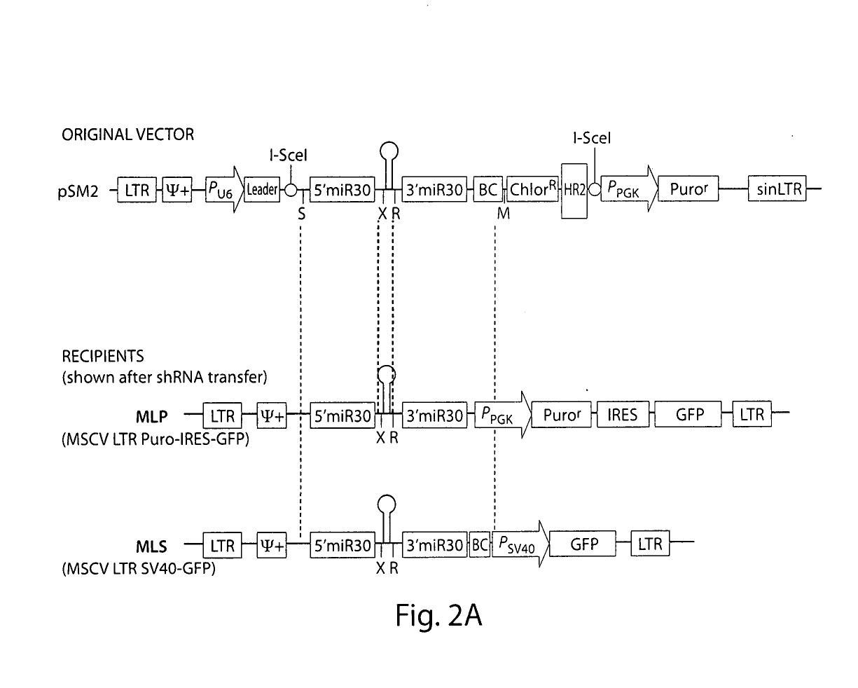

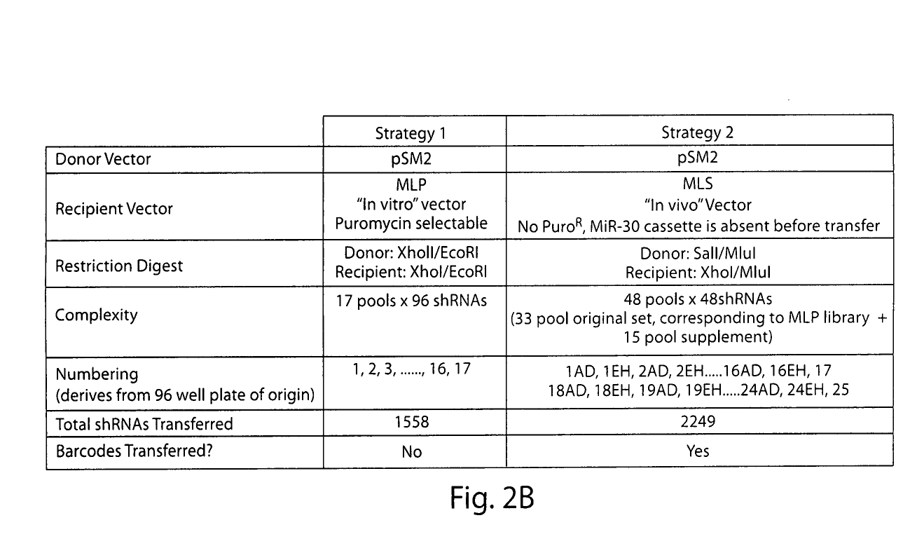

A second method to subclone the “Cancer 1000” library into recipient vectors was using restriction enzyme cut-and-paste. We transferred the cancer 1000 library, using two independent restriction cloning strategies into recipient vectors (FIGS. 2A and 2B). For simplicity, one strategy transferred only the hairpins in pools of 96 into a recipient MLP vector. MLP vector was selected because of its potent shRNA expression and its ability to be selected to homogeneity via puromycin. This selection was useful when validating individual shRNA constructs for target knockdown, used prior to western blotting. An alternative cloning strategy was to use the MLS vector as a recipient to allow both in vitro and in vivo screening. In this strategy, both hairpins and barcodes were transferred to aid future microarray readout of representation. There were 1558 total shRNAs transferred to recipient vectors using strategy 1, and 2249 shRNAs transferred using strategy 2.

Sequence-based and microarray-based quality control was performed on the subcloned pools to test for representation, revealing that the vast majority of constructs were indeed successfully transferred (data not shown).

### Example 3

### A Rapid RNAi Enrichment Screen to Identify Mediators of Doxorubicin Resistance

The Cancer 1000 shRNA set, generated in Examples 1 and 2, was used to screen and identify mediators of the response to chemotherapy. Chemotherapy resistance is not merely caused by defects in the apoptotic or senescence response to chemotherapy, but encompasses all of the processes from cellular drug metabolism and bioavailability, drug target accessibility right through to the final execution of the cellular outcome. All of these relevant factors may be probed using a genuine therapy-based screen. Here, due to relative simplicity, the primary focus was on positive selection screens, i.e., finding gene knockdowns that cause resistance to chemotherapy.

Our initial screening for shRNAs capable of conferring doxorubicin resistance was carried out in a murine Eμ-Myc Arf^{-/-} lymphoma system, which retain the p53 tumor suppressor and an intact DNA damage response (Schmitt et al., 2002). The Eμ-Myc lymphoma system has been a highly tractable model for studying the genetic determinants of chemotherapeutic response in vivo in an immunocompetent setting. These cells respond reproducibly to low doses of doxorubicin with a robust and rapid apoptotic response within 24 hours (IC50≈7 ng/ml, 16 nM). The deletion of the p19^{Arf} tumor suppressor gene uncouples the proliferative signalling via Myc from the cellular apoptotic response, thus eliminating further selective pressure for lesions in p53 or other components of the DNA damage response, which remain intact during tumorigenesis. This sensitive system is therefore useful for studying which RNAi-induced lesions can abrogate the response to doxorubicin. Murine systems are more similar to human tumors than simpler experimental organisms such as S. cerevisiae or C. elegans. Advantages of murine tumor models over human tumor cell lines include facile in vivo consolidation via allograft transplantation into immunocompetent recipients. Here, lymphoma cells home to the lymph nodes and form bona fide lymphomas, in contrast to human xenograft models. In addition, murine tumors can be engineered to be driven by a variety of defined genetic lesions for comparison across different genotypes.

shRNA pools were introduced into p19^{ARF-/-}; Eμ-Myc lymphoma cells by retroviral transduction, and infected cultures were treated with doxorubicin at doses that typically would kill 70% to 95% of cells in 24 hours.

RNAi screenings were performed according to the following protocols. Eμ-Myc; Arf^{-/-} lymphoma cells, 2 days post infection with shRNA libraries (infected to approximately 30%) were treated for 24 hours on day 1 post-infection with 7.8 ng/ml and 15.6 ng/ml doxorubicin for lenient and stringent selection conditions, respectively. 90% of the culture was removed and replaced with fresh B cell medium on day 2 and day 5 post-infection to allow recovery and proliferation of surviving cells. Final samples were taken on day 8 for GFP competition assay/shRNA representation determination. Pool-by-pool screens (FIG. 3A) were performed in a 12-well format using 500,000 cells per experimental condition (pool sizes 96 or 48 shRNAs). The single treatment, whole Cancer 1000 library screen (FIG. 3B) was performed in three biological replicates, using 1 million live, infected cells per treatment. Serial enrichment screening (FIG. 3C) was performed by infecting 1×10⁷ cells with the entire Cancer 1000 shRNA library to a final infection rate of approximately 20%. Unsorted populations of infected cells were treated for 24 hours with 7.8 ng/ml doxorubicin and then surviving cells were allowed to regrow for 4 days in fresh media. shRNAs from GFP-sorted surviving cells were recloned into the LMS parent vector and used to infect naïve lymphoma cells. This process was repeated until GFP enrichment was detectable acutely (at 24 hours) following doxorubicin treatment. This occurred consistently after 3 rounds of treatment.

To identify constituent shRNAs, genomic shRNA integrants were amplified for subcloning and validation using an identical procedure as for de novo shRNA generation (above), replacing the oligonucleotide template with genomic DNA template. Constituent shRNAs were identified using the following MSCV-specific 5′ primer: CCCTTGAACCTCCTCGTTCGACC (SEQ ID NO:1)

Three independent approaches were used to identify shRNAs enriched following doxorubicin treatment (FIG. 3). Specifically, the library was screened using either A) single treatments of lymphoma cells transduced with low complexity shRNA pools or, alternatively, B) single or C) serial treatments of lymphoma cells transduced with the whole shRNA set. For example, in one experiment, we performed a rapid positive selection enrichment screen for shRNAs-mediated resistance to doxorubicin using serial treatment (FIG. 3C). Eμ-Myc Arf^{-/-} lymphoma cells in vitro were infected with the cancer 1000 shRNA set and treated with doxorubicin. Genomic DNA was isolated from surviving cells; shRNAs were amplified from the provirus and subcloned into DNA plasmids. This cycle was repeated three times, resulting in enriched “Pool A,” “Pool B,” and “Pool C.” Enriched Pool C were collected and sequenced. The repeated PCR and subcloning steps ensured that enrichment was for relevant shRNAs rather than cellular mutants (e.g., p53) that might become enriched by serial treatment alone. DNA sequencing was used to analyze the representation of shRNA constructs in the final enriched pool.

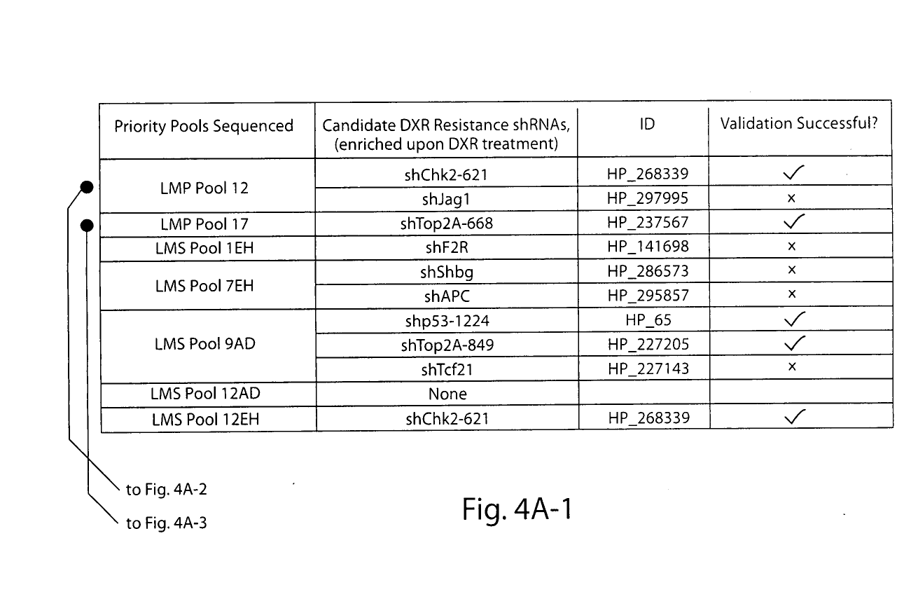

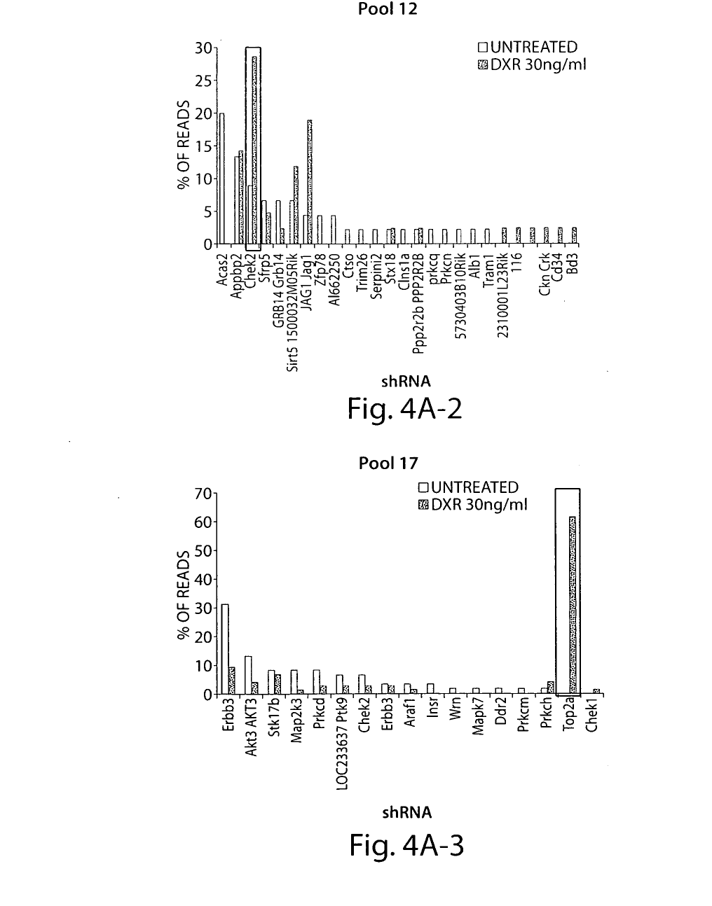

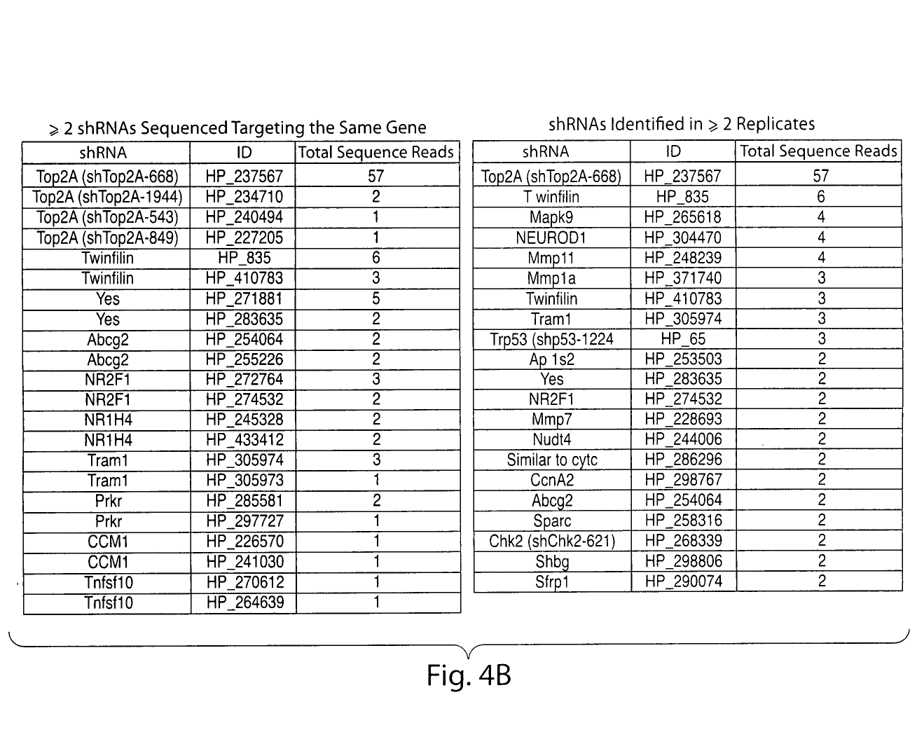

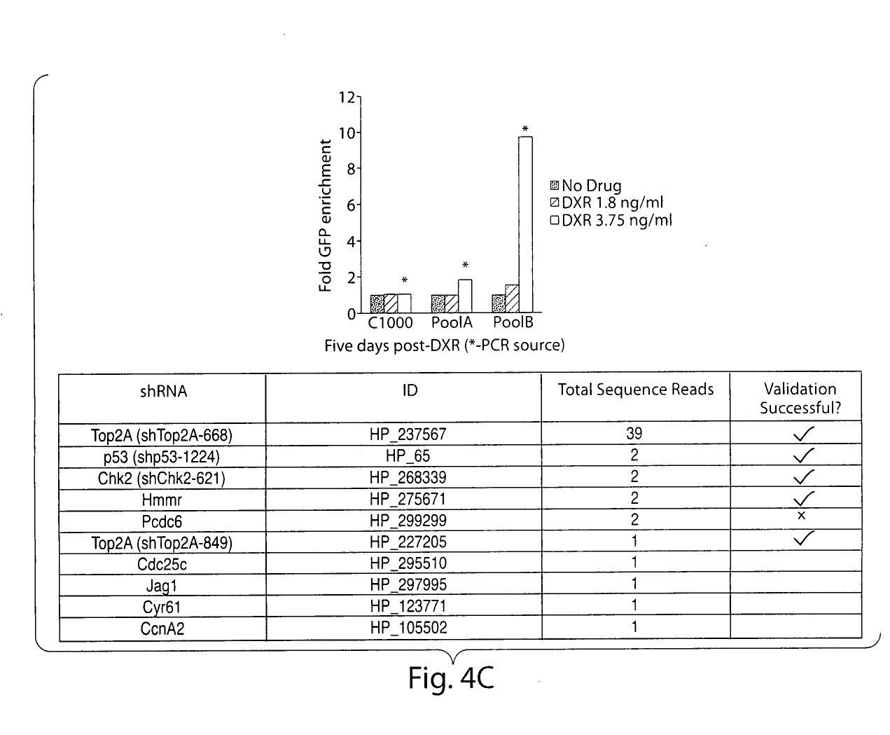

We used standard DNA sequencing of amplified provirus shRNAs to identify constituent shRNAs and to determine their relative representation in the treated and untreated cell populations (FIG. 4). Similar results were also produced using high-throughput shRNA deconvolution via DNA microarrays and Solexa deep sequencing, illustrating the potential for pooled screens of expanded scope (data not shown). Irrespective of the screening approach, shRNAs targeting p53, Chk2 and Top2A (2 independent shRNAs) were repeatedly identified as being enriched upon doxorubicin treatment. Additional shRNAs were also identified as becoming enriched following drug treatment via one strategy or another (FIG. 4), and these will be the subject of future studies.

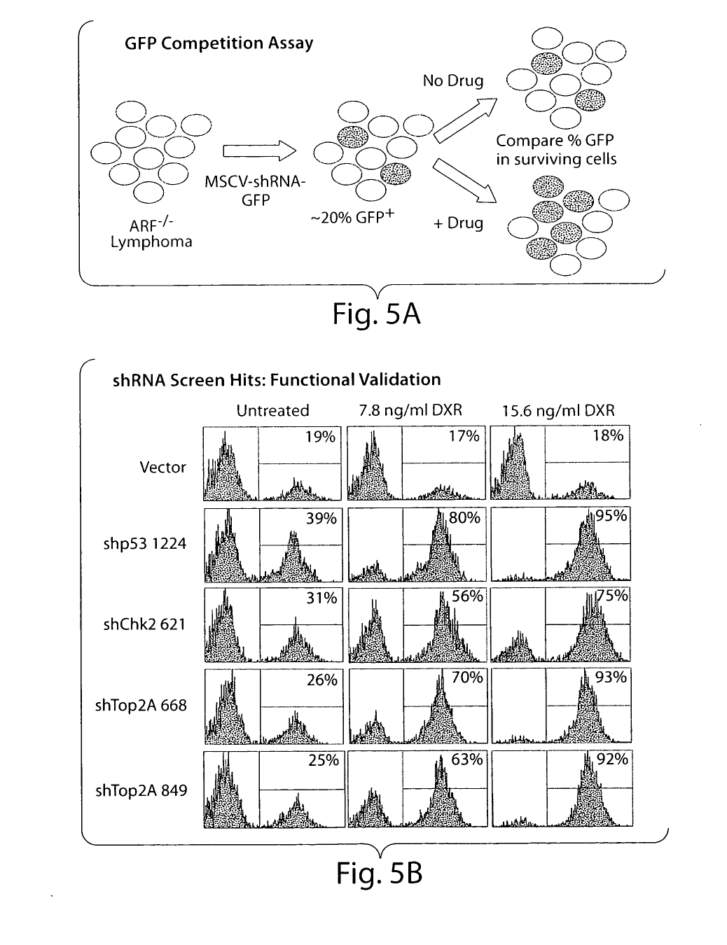

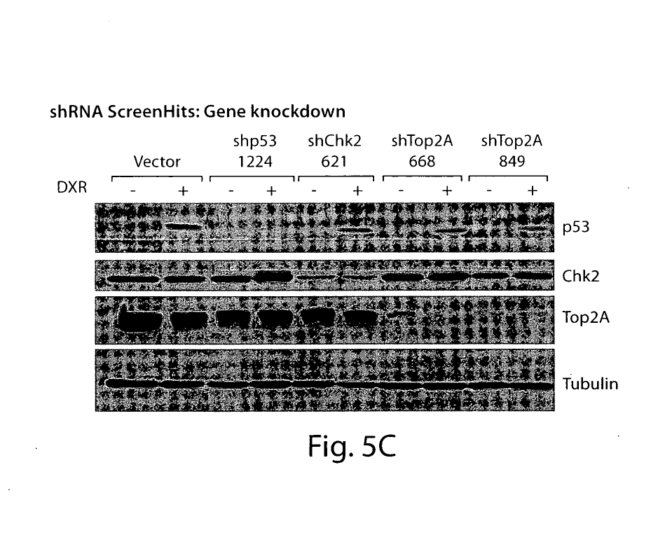

To validate the screening results and to determine whether individual shRNAs or shRNA pools affected the response to doxorubicin, we employed a sensitive in vitro GFP competition assay. This assay examines the impact of specific shRNAs on therapy response in partially-transduced cell populations, using GFP-based flow cytometry to track the survival advantage or disadvantage of conferred by specific shRNAs (FIG. 5A). shRNAs that cause resistance or sensitization to chemotherapy, as compared to uninfected cells (no fluorescence), can result in an enrichment or depletion, respectively, in percentage of GFP-containing cells in the mixed population of cells surviving chemotherapy, as determined by flow cytometry.

The effects of shRNAs targeting p53, Chk2 and Top2A were validated individually in the competition assay and western blotting: the shRNAs were dramatically enriched in cell populations within 24 hours following doxorubicin treatment (FIG. 5B); additionally, these shRNAs effectively suppressed expression of their intended target (FIG. 5C).

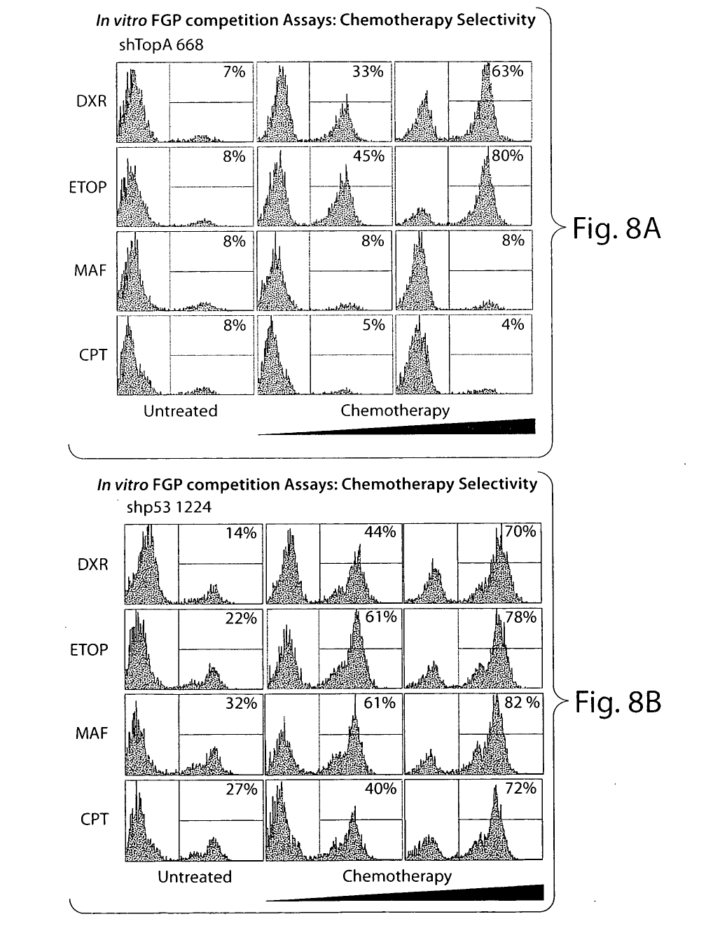

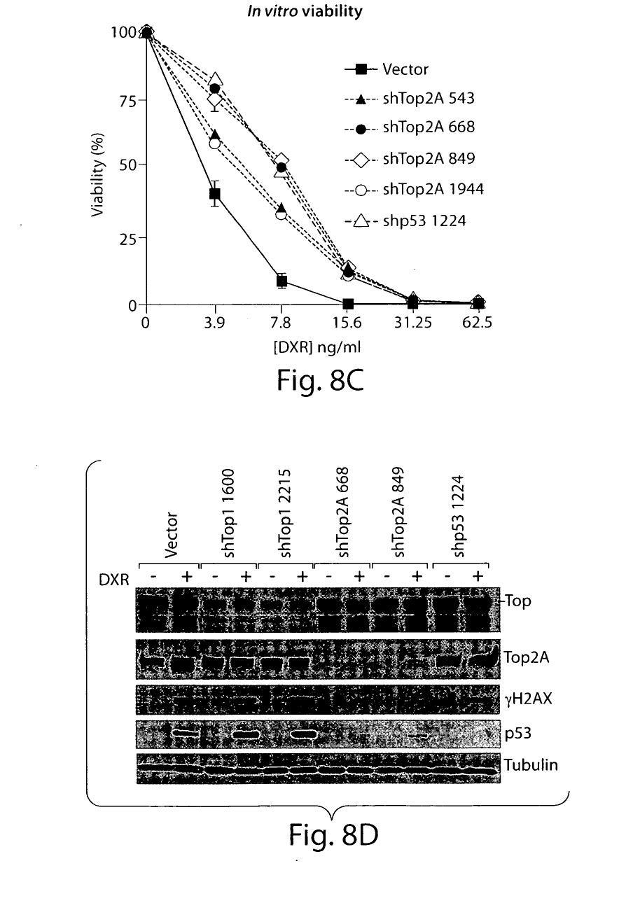

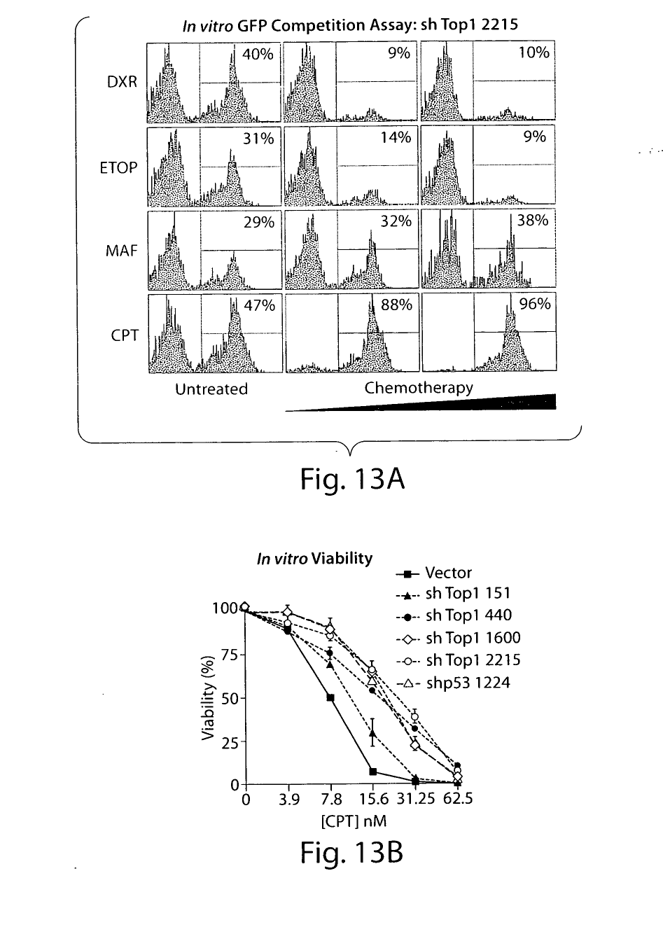

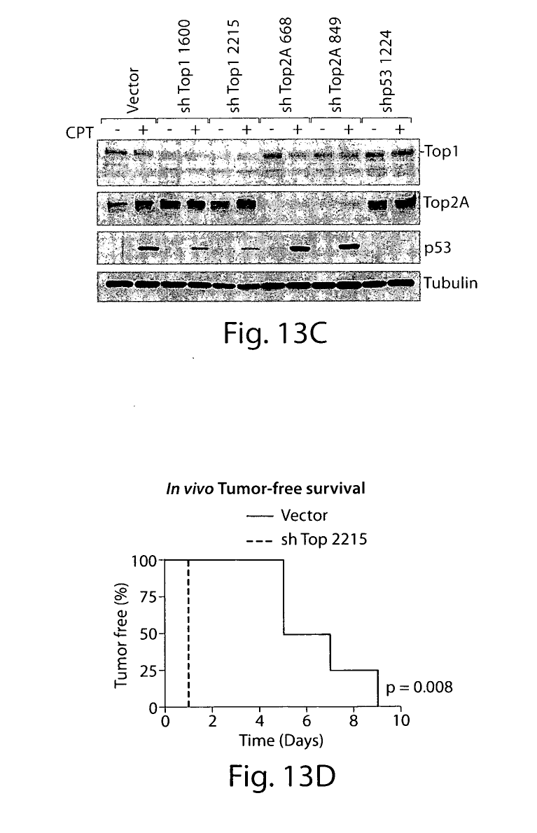

Western blotting was performed according to the following protocols. Proteins were detected using the following antibodies: anti-p53 (Clone 505, Novacastra, 1:500); anti-CHK2 (Clone 151-176, in-house monoclonal, 1:100); anti-TOP1 (Human scleroderma serum, Topogen, 1:1000); anti-TOP2A (Rabbit polyclonal, Topogen, 1:1000); anti-γH2AX (Monoclonal clone JBW301, Upstate/Millipore, 1:1000) and anti-tubulin (B5-1-2, Sigma, 1:5000). Secondary antibodies were horseradish peroxidase-conjugated anti mouse/rabbit/human IgG (GE Healthcare, 1:5000). p53 was stabilized using 31 ng/ml doxorubicin, 8 hours (FIG. 5C), 16 ng/ml doxorubicin, 8 hours (FIG. 8D) or 31 nM camptothecin, 8 hours (FIG. 13C).

Competition and viability assays were performed according to the following protocols. 2 days post infection, lymphoma cells were split into replicate wells of 500,000 cells in 12 well plates. Following 24 hour treatments with a range of drug doses, the GFP positive percentage was quantified in the surviving cell population using a BD LSRII flow cytometer. The live cell population was gated via a forward scatter (FSC) versus side scatter (SSC) plotting. For in vivo competition assays, lymphoma cells were infected in vitro, as described above. Lymphoma cells, GFP⁺ FACS sorted or unsorted, as indicated, were tail-vein injected into syngeneic recipient mice. Upon tumor onset (day 0), mice were treated with doxorubicin (10 mg/kg intra-peritoneal injection) or irinotecan (CPT-11, 50 mg/kg intra-peritoneal injection, daily for two days) and monitored for overall survival and tumor-free survival. Isolation of lymphomas for the GFP competition assay was carried out as described. For in vitro cell viability assays, lymphoma cells were treated in triplicate at the indicated doses of doxorubicin/camptothecin. Viability was determined after 24 hours by an FSC versus SSC gate and plotted relative to untreated viability.

p53 and Chk2 are key components of DNA damage response pathways and are hence satisfying proof-of-principle hits. p53 loss-of-function is generally accepted to cause resistance to DNA damaging agents in vitro (Lowe et al., 1993) and in vivo (Lowe et al., 1994; Aas et al., 1996). p53 loss also confers resistance to doxorubicin in the Eμ-Myc transgenic model.

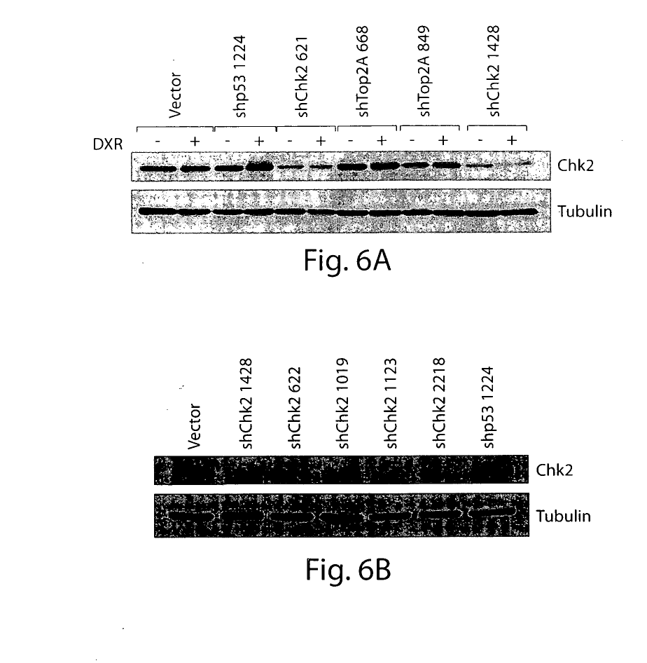

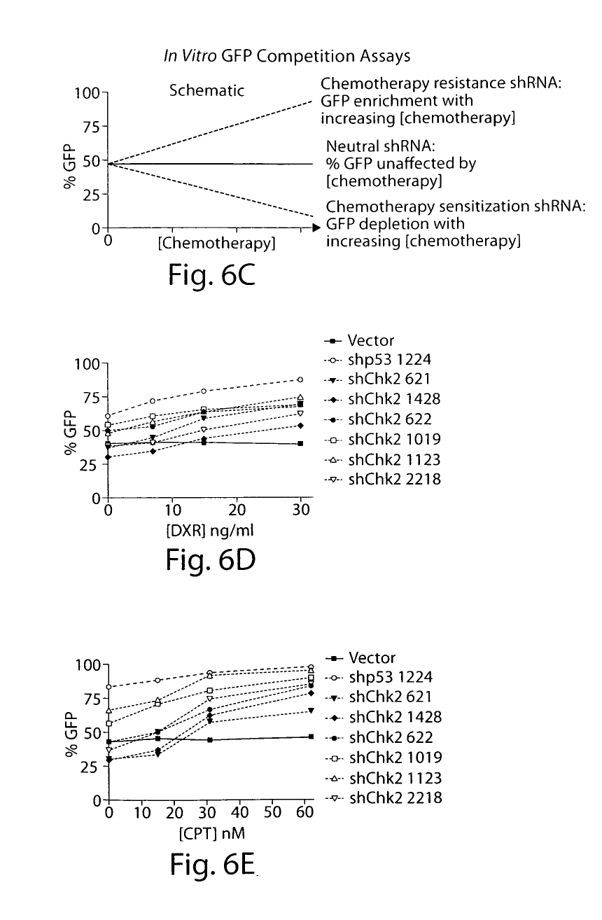

Protection from apoptosis upon chk2 loss has been primarily reported in systems where double stranded DNA breaks (DSBs) are induced by γ-irradiation (Takai et al., 2002; Hirao et al., 2002). In this experiment, sh-p53 potently knocked down p53 expression (FIG. 5C) and was not protective in a p53 null background (FIG. 8E). Importantly, multiple shRNAs targeting Chk2 promoted doxorubicin resistance, suggesting that the effects of these shRNAs were “on target”—i.e. specifically due to Chk-2 gene knockdown (FIG. 5B, FIG. 6). Although Chk2 can sensitize cells to DNA damaging agents in some contexts, our results are consistent with a role for Chk2 in signaling p53-dependent apoptosis in lymphoid cells. These results suggest we can identify relevant mediators of drug resistance using pool-based RNAi screening approaches.

### Example 4

### TOP2A Downregulation Causes Resistance Specifically to Topoisomerase 2 Poisons

shRNAs targeting Topoisomerase 2α (Top2A) were the most frequently recovered shRNAs from doxorubicin treated cells, with at least 2 independent shRNAs isolated per screen. TOP2A is the primary target of the drug doxorubicin (Fortune and Osheroff, 2000) and is an essential gene in mammals (Akimitsu et al., 2003). Unlike standard inhibitors where knocking down of the drug target would be expected to cause extra sensitization to the drug, doxorubicin is a topoisomerase “poison” which acts to stabilize the cleavable complex consisting of double stranded DNA breaks to which the enzyme is covalently attached. Doxorubicin therefore causes excessive double stranded DNA breaks via unresolved cleavable complexes, in a topoisomerase-dependent manner, explaining why TOP2A downregulation causes doxorubicin resistance. Remarkably, even very potent knockdown of Top2A (FIG. 5C) had little, if any, impact on cell proliferation in the absence of drug treatment, suggesting that normal cell proliferation can proceed with relatively low Top2A expression (data not shown).

Although previous work has suggested a relationship between Top2A levels and doxorubicin sensitivity, the effect has not been studied extensively or validated in vivo. As TOP2A was our strongest hit in the screen, we decided to investigate more fully the effect of TOP2A downregulation on drug response. To control for potential off-target effects of TOP2An shRNAs, we generated a total of four TOP2An shRNAs and found them to all potently cause doxorubicin resistance, as shown by GFP competition assay and in vitro survival curves (FIGS. 7 and 8).

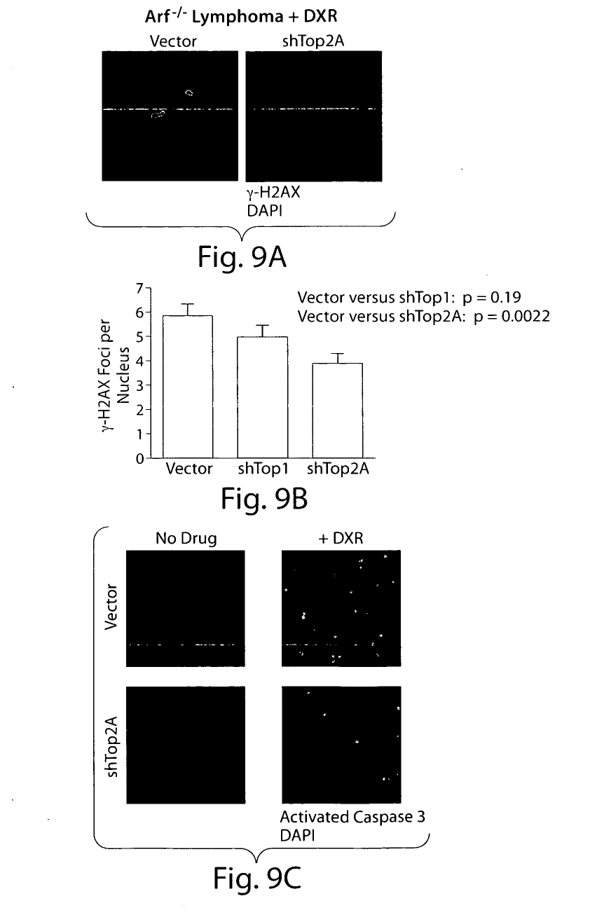

The effects of Top2A knockdown were specific to topoisomerase 2 poisons: shTop2A causes resistance to another, structurally unrelated, TOP2A poison, etoposide, but not to the alkylating agent maphosphamide (an active metabolite of cyclophosphamide) nor the topoisomerase 1 poison camptothecin (FIG. 8A). In contrast, an shRNA targeting p53 causes cross-resistance to these different agents (FIG. 8B). The drug response modifying effects of Top2A knockdown are likely ‘on target’: four out of four Top2A shRNAs mediate resistance specifically to topoisomerase 2 poisons, as demonstrated by a significant increase in the percentage of GFP-containing cells, and the corresponding increase in cell survival rate upon DXR treatment (FIG. 7, FIG. 8). Consistent with TOP2A being the drug target of doxorubicin, the mediator of doxorubicin DSBs and upstream of the DNA damage response, rather than a more general transducer of DNA damage signals, cells with reduced TOP2A levels displayed a diminished DNA damage signal and response as compared to controls, as shown by lower γ-H2AX signal, less p53 stabilization and less apoptosis, upon doxorubicin treatment (FIG. 8D, FIG. 9). Accordingly, the ability of Top2A shRNAs to promote doxorubicin resistance was attenuated in p53 null Eμ-Myc lymphoma cells (FIG. 7B), although clearly some signals downstream of chemotherapy-induced DNA damage are p53-independent.

The effect of TOP2A knockdown on doxorubicin sensitivity was not unique to this Eμ-Myc Arf^{-/-} lymphoma model; similar results were shown in Eμ-Myc p53^{-/-} lymphoma cells (FIG. 7B), murine acute myeloid leukaemia (AML) cells (data not shown) and HeLa cells (Gudkov et al., 1993).

Additionally, TOP2A knockdown caused resistance specifically to topoisomerase 2-targeted poisons. shTOP2A caused resistance to another TOP2A poison, etoposide, but not to the topoisomerase 1 poison, camptothecin, or the alkylating agent maphosphamide (an active metabolite of cyclophosphamide). In contrast, sh-p53, caused cross-resistance to all these agents (FIG. 7C).

### Example 5

### Top2A shRNAs Confer Resistance to Doxorubicin In Vivo

Currently, attempts to link TOP2A expression levels to doxorubicin sensitivity have principally compared genomic copy number or expression levels of TOP2A in either clinical tumor samples of different clinical doxorubicin responses or, alternatively, cells selected continuously in vitro for doxorubicin resistance relative to their more sensitive parental cell line. Such correlative studies are complicated by additional genetic differences between the comparison samples and report conflicting results, both in vitro (Arriola et al., 2006; Pang et al., 2005; Yasui et al., 2004) and in vivo (Villman et al., 2006; Tanner et al., 2006; Arriola et al., 2006). The assumption that TOP2A genomic amplification necessarily results in increased gene expression has also been questioned (Mueller et al., 2004). Results may be further complicated by the close proximity of the chromosomal loci of TOP2A and ErbB2, which likely have opposite effects on sensitivity to anthracyclines. (Hu et al., 2006). Normalizing for ErbB2 status has revealed that tumors with doubly amplified TOP2A/ErbB2 have a favourable response to anthracyclines compared to ErbB2 singly amplified tumors (Tanner et al., 2006). Acute TOP2A knockdown is therefore a powerful technique to dissect the role of TOP2A levels on doxorubicin sensitivity on an isogenic background and has not previously been attempted in vivo.

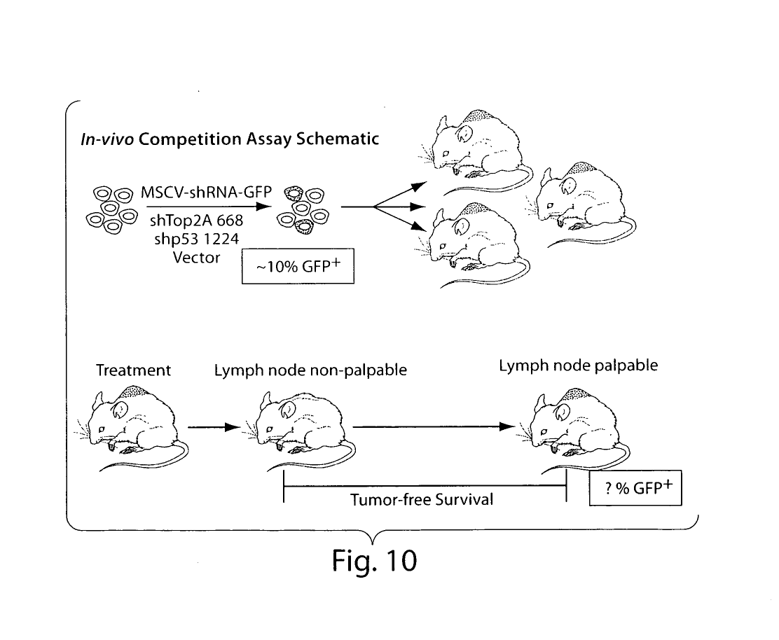

To test the role of Top2A in doxorubicin resistance in vivo, Eμ-Myc; Arf^{-/-} lymphoma cells were infected in vitro with shTop2A or a control vector and transplanted via tail vein injection into multiple syngeneic recipient mice. Tumor-bearing recipient mice were then treated with the maximum tolerated dose of doxorubicin at tumor onset (FIG. 10).

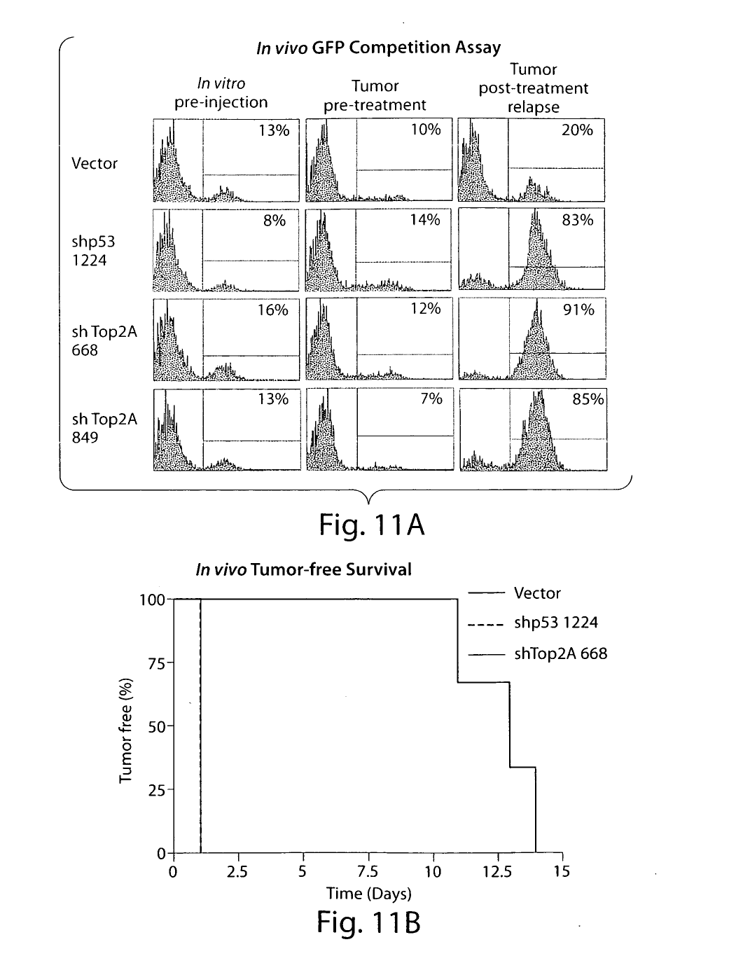

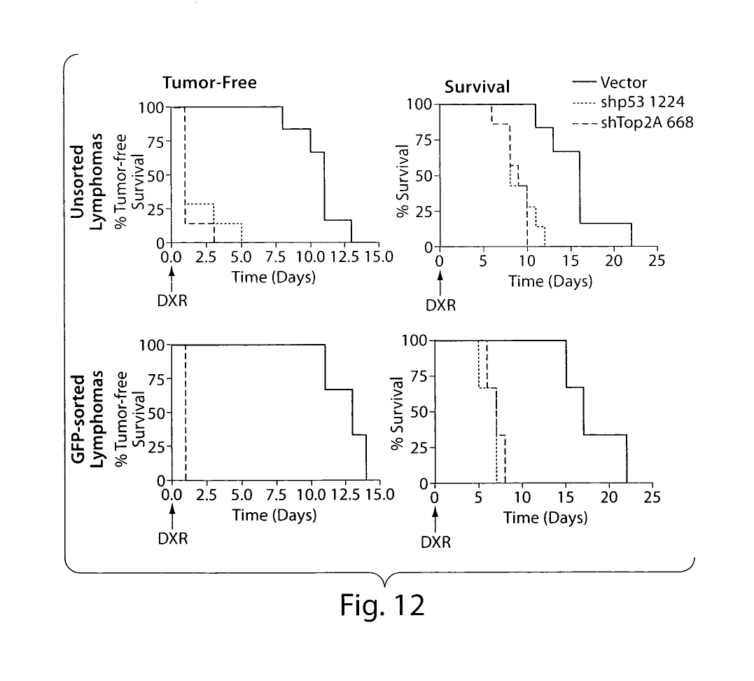

TOP2A knockdown caused doxorubicin resistance in vivo as measured by in vivo competition assay (an increase in the % GFP-positive cells following drug treatment, FIG. 11A), by reduced tumor free survival (FIG. 11B), and by overall survival (FIG. 12). Survival differences between shTOP2A and vector control tumors were enhanced if cells were FACS sorted to GFP⁺ (infected) homogeneity prior to transplantation (FIG. 12). These results demonstrate that reduced Top2A expression is a bona fide mechanism of drug resistance in vivo.

### Example 6

### TOP1 Down-Regulation Causes Resistance to Topoisomerase 1 Poisons In Vitro and In Vivo

TOP2A is not unique as a topoisomerase target of clinically important anti-cancer therapeutics. Topoisomerase 1 (TOP1) is the target of camptothecin (Hsiang and Liu, 1988; Hsiang et al., 1985) and its derivatives such as irinotecan (camptosar/CPT-11), approved for treating colorectal carcinoma, and topotecan (hycamtin), approved for treating ovarian, cervical and small cell lung cancer. TOP1-deficient yeast are viable and resistant to camptothecin (Nitiss and Wang, 1988; Yasui et al., 2004), but, as for TOP2A, complete knockout is embryonic lethal in mammals (Morham et al., 1996).

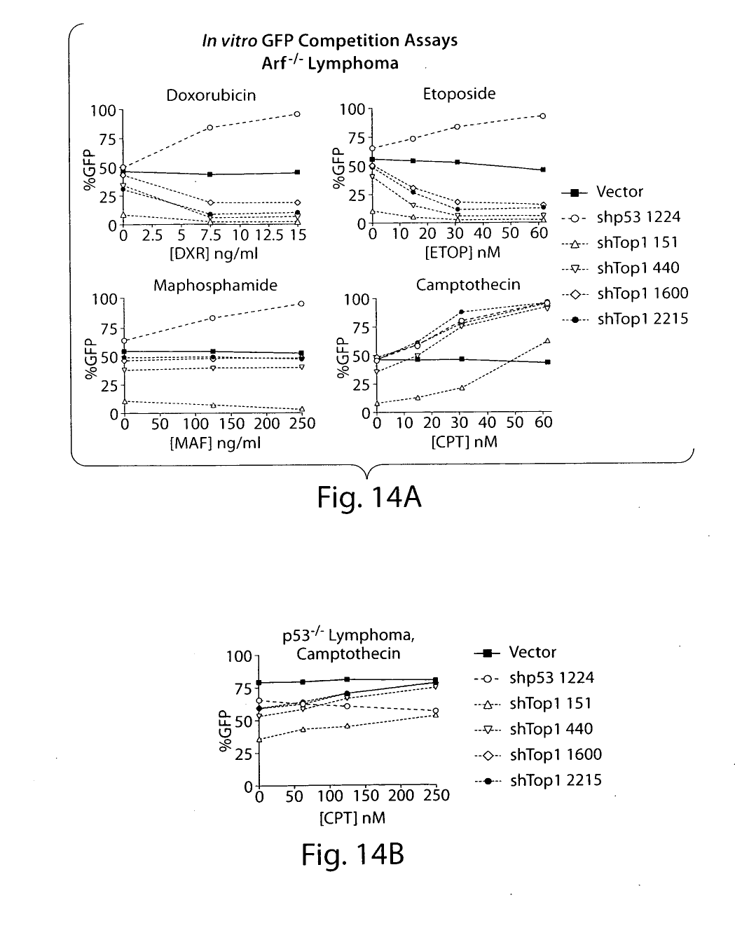

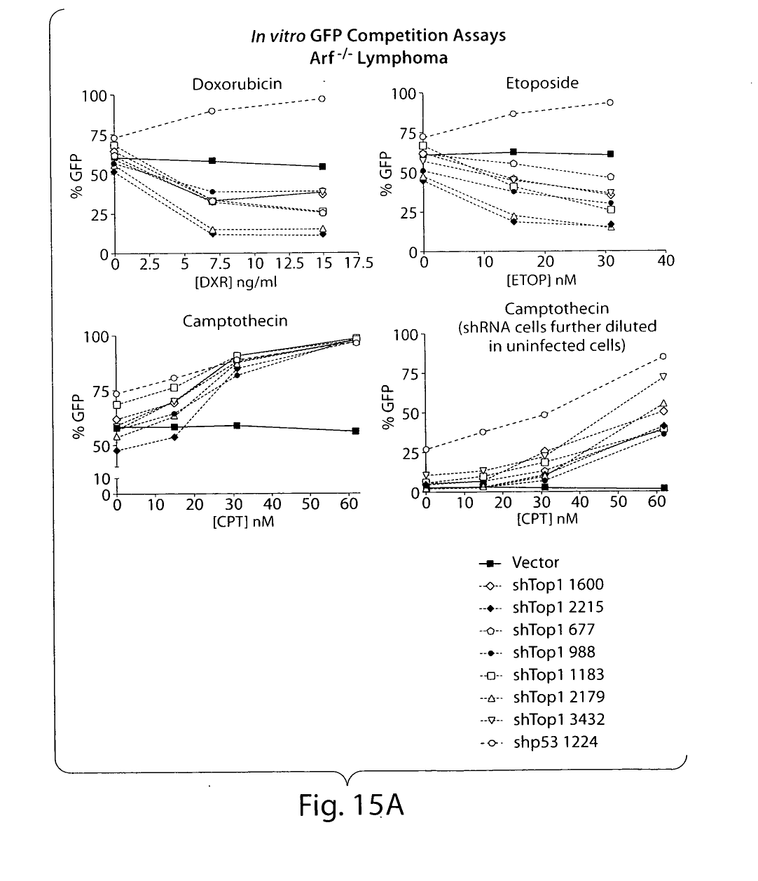

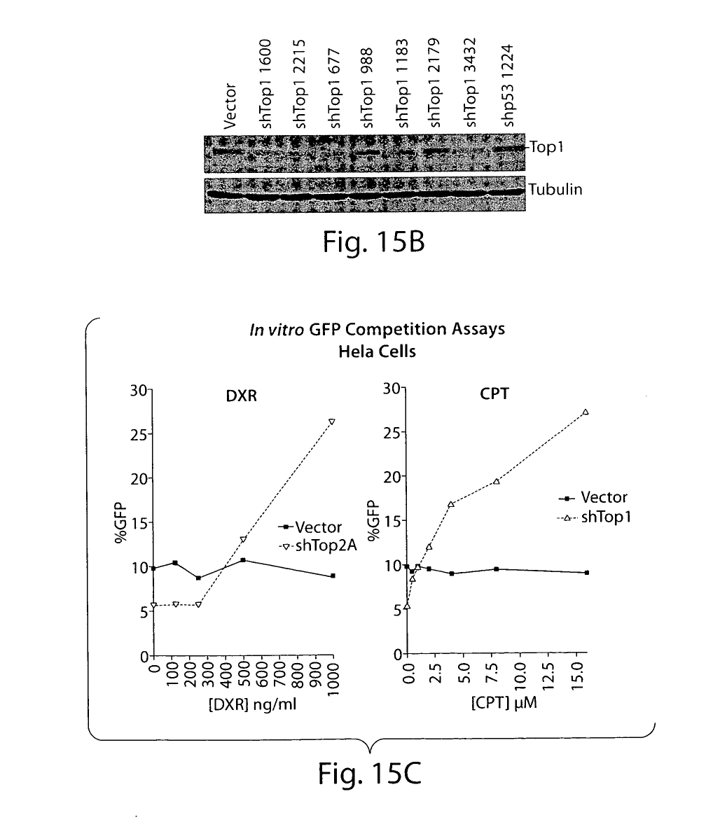

Prompted by our studies on doxorubicin and Top2A, we tested whether Top1 knockdown could induce camptothecin resistance in cancer cells. Analogous to the effect of TOP2A expression levels on doxorubicin sensitivity, TOP1 knockdown caused camptothecin resistance. Indeed, Top1 knockdown in Eμ-Myc; Arf^{-/-} lymphomas causes resistance specifically to camptothecin (FIG. 13A; see also GFP competition assays, FIGS. 14 and 15, and viability curves, FIG. 13B). The effects were reproducible using multiple independent Top1 shRNAs (FIG. 13B, FIGS. 14 and 15), as all four TOP1 shRNAs caused a significant increase in the percentage of GFP-containing cells, and the corresponding increase in cell survival rate upon camptothecin treatment. Even modest Top1 knockdown achieved this cytoprotective effect (see, Western blot analysis, FIG. 13C). Importantly, this effect was also seen in human cells expressing a TOP1 shRNA (FIG. 15C).

Interestingly, potent camptothecin resistance was achieved despite the relatively modest TOP1 knockdown of approximately 50%, emphasising the potential relevance to patient tumors hemizygous at the TOP1 locus. Consistent with the model that topoisomerase knockdown limits the level of DNA breaks upon treatment with a poison targeting that topoisomerase, diminished p53 induction was observed upon camptothecin treatment in TOP1 knockdown settings, as compared to controls (“vector”) (FIG. 13C). This result suggests that these cells mounted a weaker DNA damage response. Accordingly, resistance was also attenuated in an Eμ-Myc; p53^{-/-} background (FIG. 14B). Mice harboring shTop1-expressing lymphomas displayed a reduced tumor free survival compared to controls following treatment with irinotecan, indicating that reduced Top1 expression promotes resistance to topoisomerase 1 poisons in vivo (FIG. 13D). Therefore, sufficient expression of Top2A or Top1 is required to achieve a potent response to chemotherapeutic agents targeting each particular topoisomerase.

As for doxorubicin treatment, p53 knockdown caused resistance to camptothecin, illustrating that both a decrease in DNA damage via topoisomerase knockdown, or a block to the apoptotic response to damage can result in therapy resistance. In an Eμ-Myc p53^{-/-} background, all four shTOP1, but not sh-p53, resulted in camptothecin resistance (FIG. 14), illustrating that while much of the doxorubicin or camptothecin-initiated DNA damage signals through the p53 pathway, there are p53-independent pathways to apoptosis and that topoisomerase expression levels may be relevant to therapy outcome in a variety of tumor genotypes, regardless of p53 status.

### Example 7

### TOP1 Suppression Sensitized Cells To Topoisomerase 2 Poisons

The drug resistance phenotypes conferred by Top1 shRNAs were specific for topoisomerase 1 poisons. For example, Top1 knockdown had little effect on tumor cell sensitivity to the alkylating agent maphosphamide (FIG. 13A).

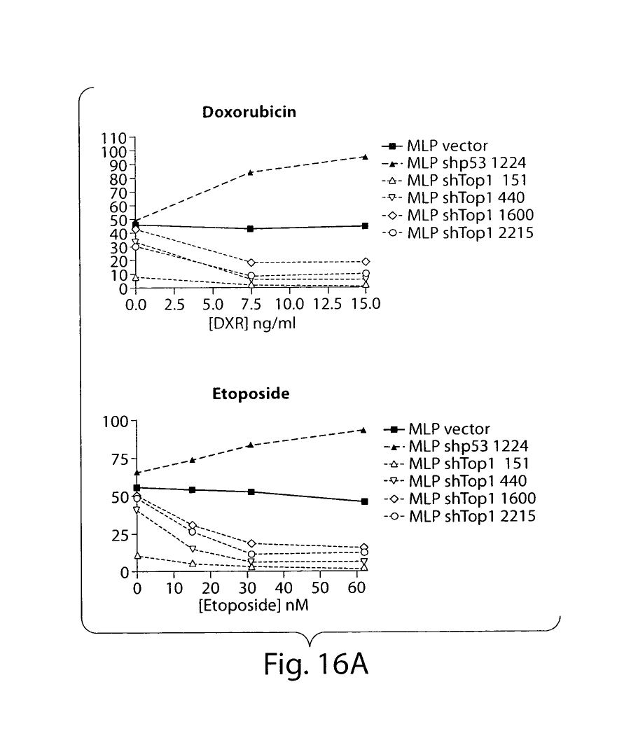

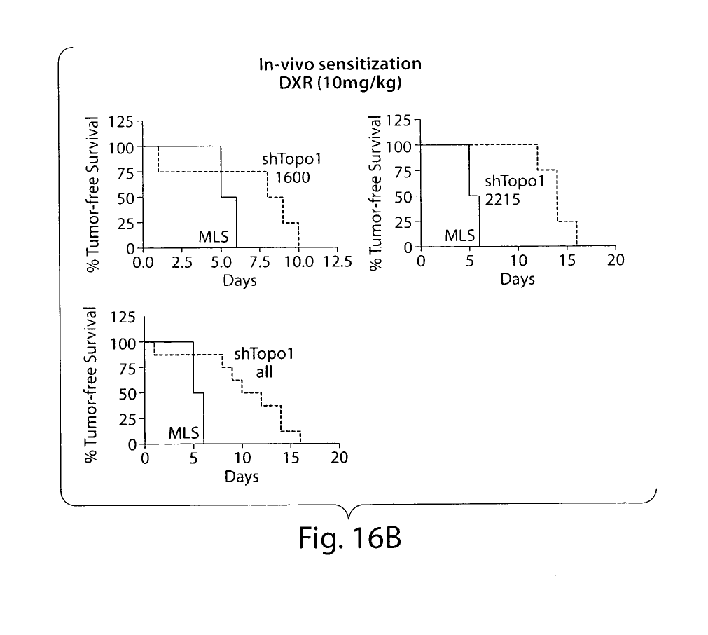

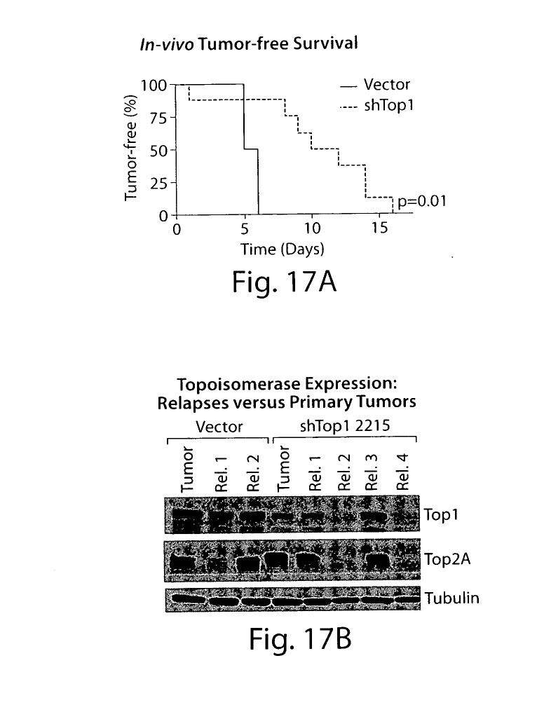

In testing the effects of TOP1 knockdown on the response to a variety of chemotherapeutic drugs, we noticed that not only was TOP1 knockdown-mediated drug resistance specific to TOP1 poisons but that, interestingly, TOP1 downregulation hypersensitized Eμ-Myc Arf^{-/-} lymphoma cells to the Topoisomerase 2 poisons doxorubicin and etoposide (FIG. 13A; see also, competition assay, FIG. 16A). This effect was reproduced with 9 independent Top1 shRNAs (FIGS. 14 and 15). We tested this result again using the lymphoma transplantation and the in vivo treatment method. Here, TOP1 knockdown successfully hypersensitized Eμ-Myc Arf^{-/-} lymphomas to doxorubicin, as mice harboring transplanted lymphomas expressing Top1 shRNAs showed an improved tumor-free survival compared to controls following irinotecan treatment (FIGS. 16B and 17A).

### Example 8

### Spontaneous Changes in Topoisomerase Levels Accompany Relapse Following Doxorubicin Therapy

The ability to manipulate tumor genotypes by RNAi is a powerful approach to delineate which genes influence the response to therapy. Our results emphasize the importance of knowing critical features of tumor genotypes in order to successfully tailor cancer therapy to the individual, and in order to predict, in advance, the most effective therapy. Based on data here, one might predict that for a tumor that has become refractory to camptothecin therapy, that of the many possible camptothecin resistance mechanisms, p53 loss would indicate that subsequent doxorubicin therapy would not be successful but, alternatively, TOP1 downregulation might make subsequent doxorubicin therapy highly effective.

In our systems, we have manipulated topoisomerase expression levels to induce resistance or sensitisation to topoisomerase poisons. We wanted to see whether such resistance mechanisms would occur in tumors in vivo, in the absence of such enforced manipulations, or whether other, non-topoisomerase, resistance mechanisms would predominate. Tumor relapses may display intrinsic or acquired therapy resistance signatures that have enabled the surviving tumor cells to overcome therapy.

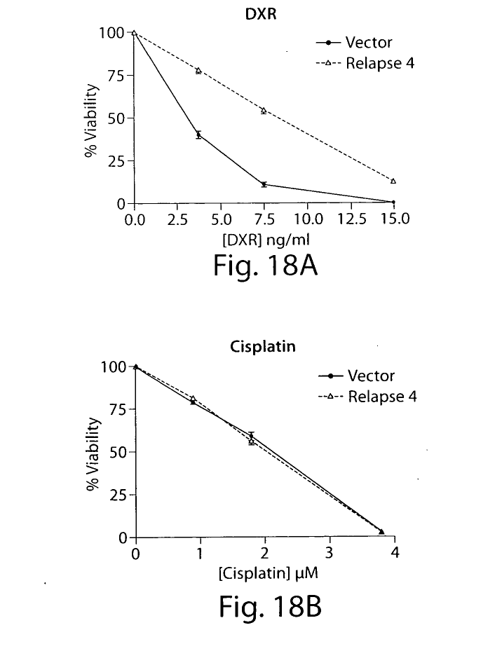

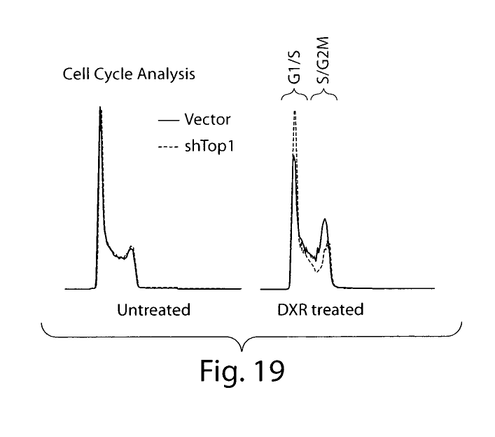

To examine the relevance of topoisomerase status to resistance mechanisms spontaneously occurring in treated lymphomas, primary tumors and post-doxorubicin treatment relapses from FIG. 17A were analyzed for Top1 and Top2A expression levels (FIG. 17B). The relevance of Top2A levels to the emergence of tumor relapses was supported by the fact that half of the relapsed tumors displayed dramatically reduced Top2A levels (1 of 2 control tumors and 2 of 4 shTop1-expressing tumors) without experimental manipulation via Top2A shRNAs. As further evidence that Top1 knockdown can sensitize to the topoisomerase 2 poison doxorubicin, one shTop1 relapse (relapse 3) recovered expression of Top1 to approximately wild-type levels. Relapsed tumors treated ex vivo showed resistance to doxorubicin, but not cisplatin, suggesting that the resistance mechanisms were topoisomerase-specific (FIG. 18). Together, these results indicate that while alterations in topoisomerase expression levels represent one of undoubtedly many therapy resistance mechanisms, these changes can play a substantial role in chemotherapy response in vivo.

We have documented the utility of combining RNAi screens with mouse cancer models to identify and characterize molecular determinants of therapeutic response that are relevant to treatment outcome in vivo. This approach is ideal for rapid in vivo validation of candidate genes, and may serve as a relevant setting for conducting in vivo RNAi based screens for genetic determinants of drug resistance. Such methodology is easily extendable to other chemotherapeutics and tumor systems to allow a more global view of therapy response mediators, including their context-dependence across different tumor and host genotypes.

### Example 9

### Comprehensive Targeting of Topoisomerase Gene Family Members

shRNAs were synthesized, targeting all mammalian topoisomerase family members, as well as a related gene, topoisomerase binding protein 1 (TopBP1). All of these shRNAs were tested in the GFP competition assay for their effects on the response to camptothecin or doxorubicin treatment (as examples of topoisomerase 1 and 2 poisons, respectively). There are no known poisons of topoisomerase 3 family members. Knocking down the expression of Top2B, Top3A, Top3B and TopBP1 did not result in significant sensitization or resistance to camptothecin and doxorubicin treatment: Top2B shRNAs only led to very mild doxorubicin resistance (data not shown); TopBP1 shRNAs mediated subtle (but reproducible) resistance to both camptothecin and doxorubicin (data not shown), consistent with its reported role in the DNA damage response (Bartek and Mailand, 2006).

By far the most prominent resistance and sensitisation effects were observed with shTOP1 and shTOP2A. The data are also consistent with TOP2A being the major target of doxorubicin.

Although RNAi is not currently a clinically-applied therapeutic method, the effects of gene knockdown can mimic the action of small molecule inhibition and, hence, RNAi can highlight new drug targets worthy of pharmaceutical development. Based on this study, therefore, catalytic inhibition of TOP1 by a non-poisoning drug, i.e., an inhibitor of TOP1 function that does not induce unresolved DNA breaks might be an effective therapy to sensitize tumors to topoisomerase 2 poisons, such as doxorubicin or etoposide. This class of inhibitor does not exist for TOP1, although the bisdioxopiperazine compounds represent a class of catalytic inhibitors of TOP2A (Andoh and Ishida, 1998). These compounds, for example ICRF-193 and ICRF-159, inhibit the ATPase activity of TOP2A (Hu et al., 2002). An identical strategy is impossible for TOP1 due to the absence of an ATPase domain, but an alternative molecular mechanism such as prevention of TOP1 binding to DNA could be valuable.

### Example 10

### Microarray-Based Method to Identify shRNAs that Mediate Drug Sensitization or Drug Resistance

In this example, high throughput methods, such as microarrays, were used to find shRNAs mediating drug sensitization or drug resistance.

The potential to investigate negatively-selected shRNAs greatly advances the therapeutic implications of screens. While positive selection screens can identify crucial pathways involved in therapy response and suggest potential resistance mechanisms, negative selection screens can truly uncover cancer cell vulnerabilities: gene knockdowns that sensitize to therapy or directly kill cells. Gene knockdown can serve as an experimental surrogate for small molecule inhibition, thus suggesting novel cancer drug targets, which may be used to develop therapeutics or to find new uses of existing drugs (Oosterkamp et al., 2006; Brummelkamp et al., 2003). Of particular interest is to find genotype-specific vulnerabilities in order to exclusively kill those vulnerable tumours known to contain the lesion(s), while limiting systemic toxicity (Farmer et al., 2005).

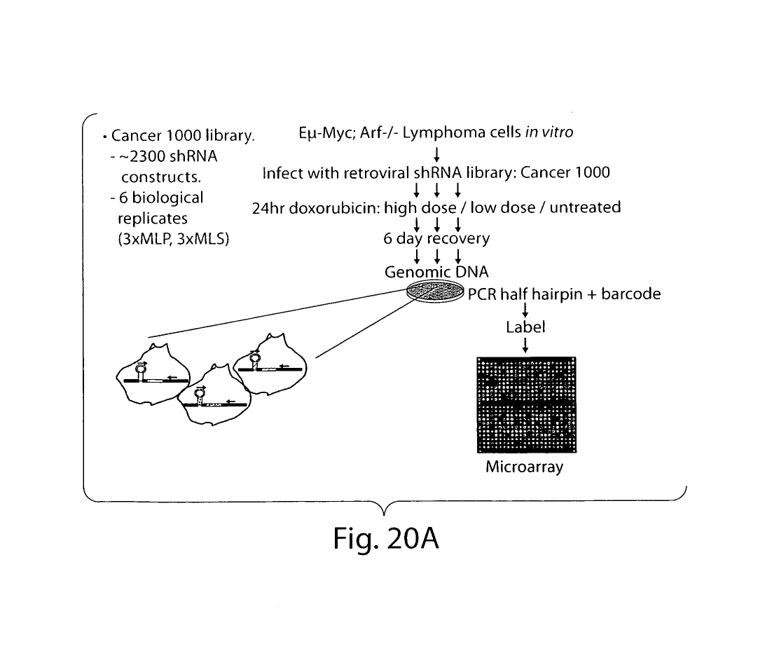

The overall experimental strategy is outlined in FIG. 20A. PCR products used in this experiments encompassed half of the hairpin and (for the MLS version) the barcode, and could be used as independent identifiers for microarray hybridization. Only half of the hairpins were amplified, in order to prevent the hairpin secondary structure (intramolecular hybridization) from inhibiting array hybridization (intermolecular). The barcode was considered a superior, more specific microarray probe due to its greater length (60 nt compared to ˜20 nt half-hairpins). However, not all shRNAs had barcodes of known sequence so both barcodes and half hairpins remained valuable shRNA identifiers.

Nimblegen custom design 12-plex microarrays were chosen as the array platform for these studies. These arrays are made up of 12 identical subarrays of 13,000×50mer features of custom sequence. Arrays typically consist of barcode sequences, two identical half-hairpin sequences in tandem, or a combination of both. In the studies reported here, the array design was entirely half-hairpin and consisted of all Cancer 1000 shRNAs (˜2300 shRNAs), with the remaining features representing other mouse-targeted shRNAs, here serving as negative hybridization controls.

Hybridizations were performed as a two-colour (cy5/cy3, experimental/reference) competitive hybridization. The experimental sample was derived from lymphoma cell genomic DNA±drug treatment. The reference sample consisted of the same PCR methodology performed on the initial plasmid DNA mixture used in the screening (prior to transfection/infection). The reference sample served as a standardization control across different arrays and subarrays. Additionally, the reference sample gave an indication of the efficiency of subcloning from pSM2 to MLP/MLS vectors, i.e. what proportion of shRNAs may have been lost during subcloning.

Array hybridisation was performed on the following samples of Eμ-Myc Arf^{-/-} lymphomas in vitro±doxorubicin (time 0=start of treatment):

1. Untreated, day 7.

2. 15.6 ng/ml doxorubicin (low dose) day 7 (24 hour treatment, 6 day recovery)

3. 31.3 ng/ml doxorubicin (high dose) day 7 (24 hour treatment, 6 day recovery, low cell number)

4. 31.3 ng/ml doxorubicin (high dose) day 10 (24 hour treatment, 9 day recovery, higher cell number)

Each sample was run in 6 biological replicates (3 in MLP vector, 3 in MLS vector).

The vast majority of “Cancer 1000” probes showed above background signal (data not shown) implying both an efficient library subcloning into MLP/MLS and a successful hybridization.

Principal component analysis of the data identified the primary sources of variability in the data (FIG. 20B). We also noted that biological replicates clustered with each other more closely than samples treated with different drug doses, illustrating good reproducibility between replicates, and meaningful changes in shRNA representation caused by doxorubicin treatment.

In addition to the above statistical analysis, we also performed a parallel analysis of the experimental samples whereby microarray features were simply ranked by absolute intensity, as an alternative form of normalization. Major changes in rankings upon drug treatment were examined.

The most potently enriched shRNAs following doxorubicin treatment were identified for the MLS and MLP replicates using both a rigorous statistical analysis (FIG. 20C) and a simple intensity ranking method (FIG. 20D). In all cases, multiple shRNAs targeting Top2A were identified as top scorers, as a satisfying demonstration that it is possible to find crucial drug resistance-mediating shRNAs via this microarray methodology, using different vector settings and alternative data analyses. Interestingly, the straightforward data analysis using intensity ranks performed equally or even in a superior fashion to the rigorous statistical analysis for this example, as indicated its ability to place Top2An shRNAs at the top of the list (previous screens indicate that these were genuinely the most potent hits) and to find a further (weaker) positive control, shChk2-621, which the statistical analysis did not.

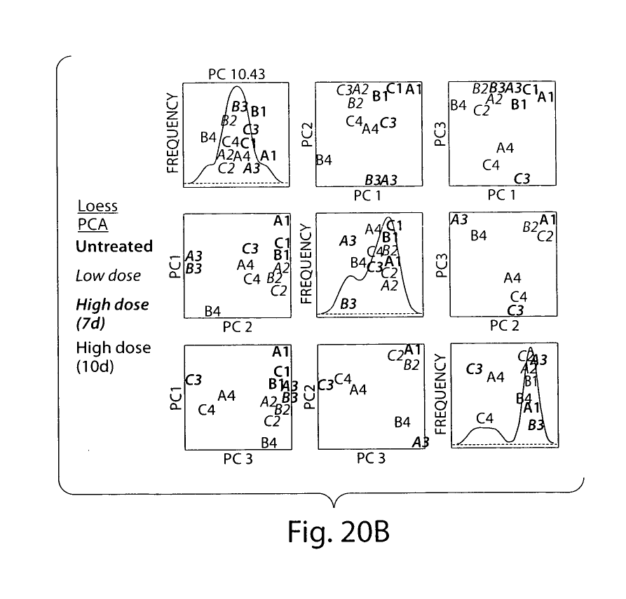

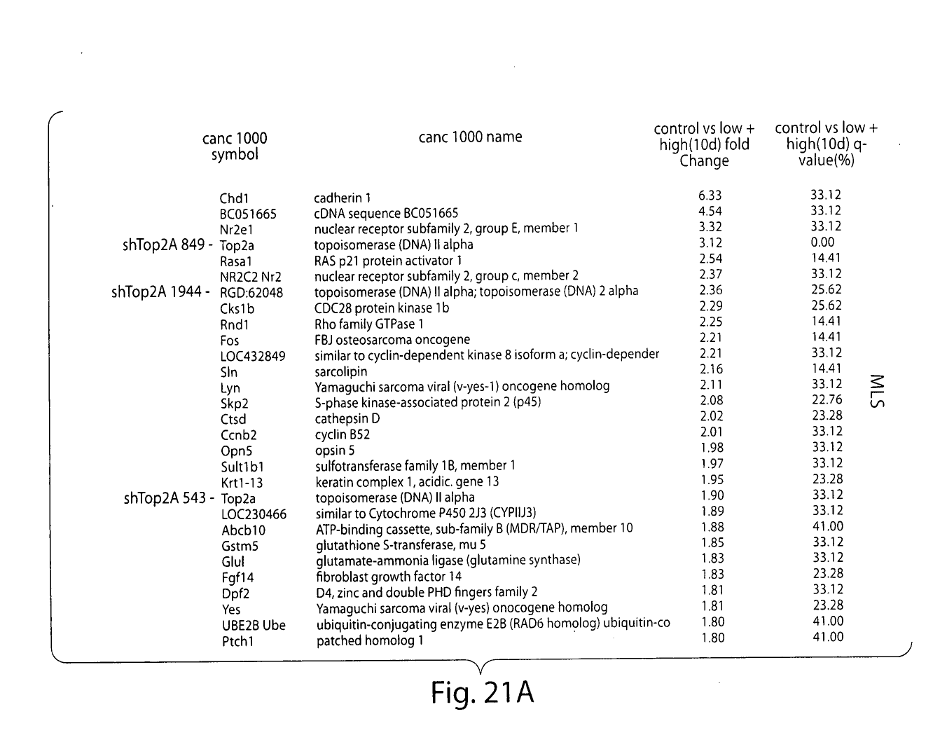

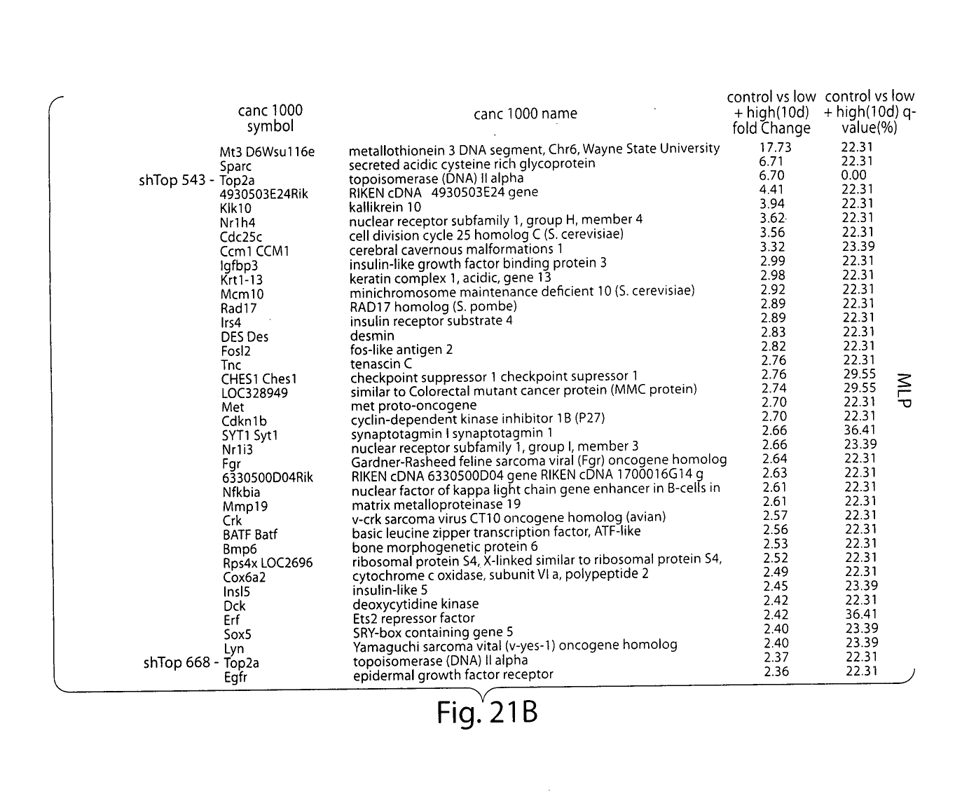

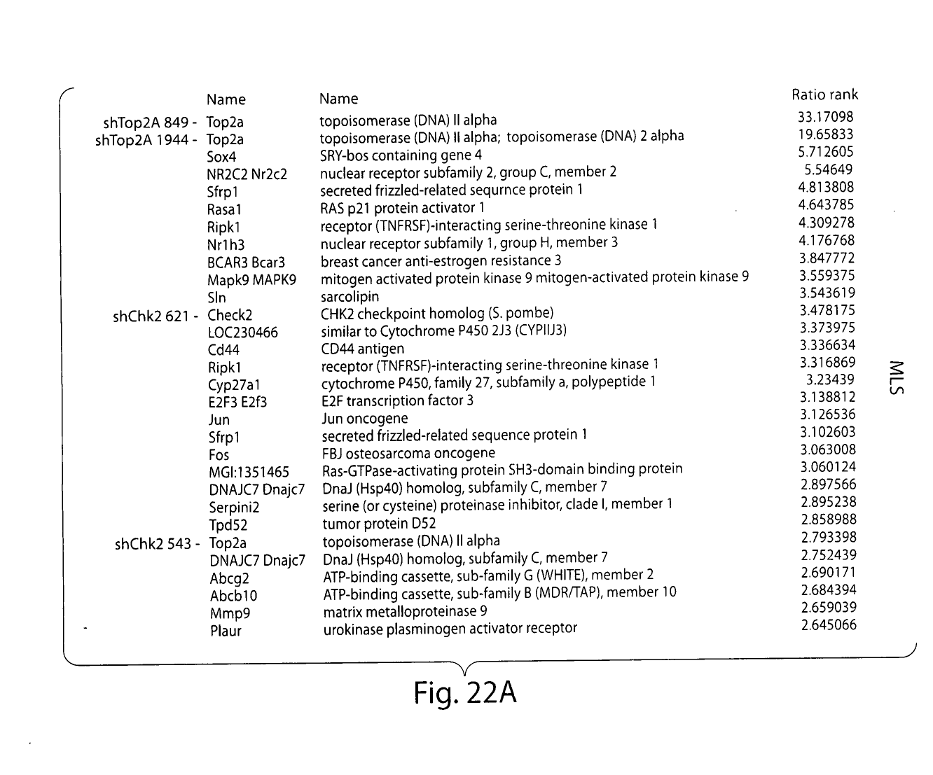

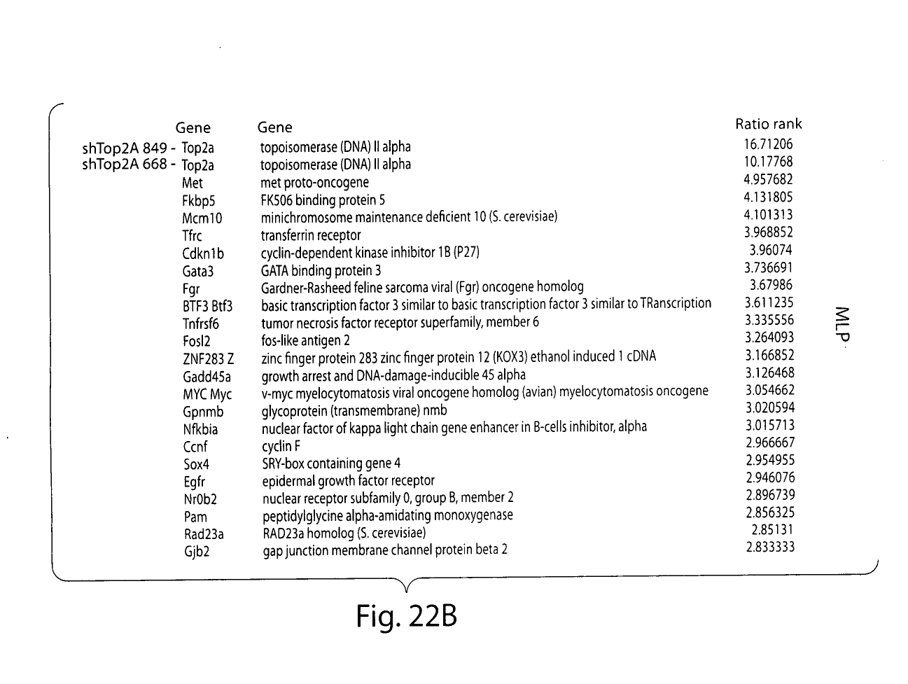

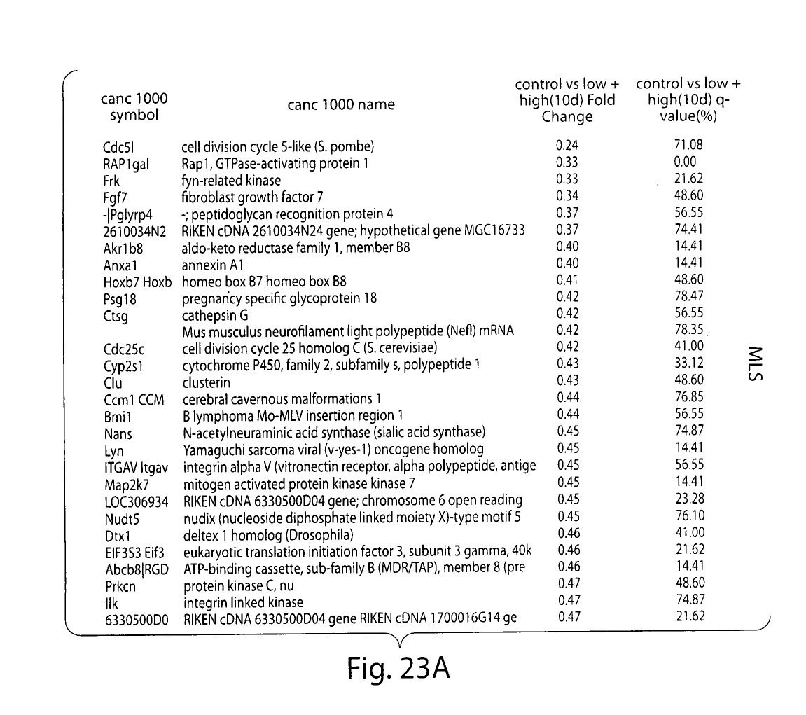

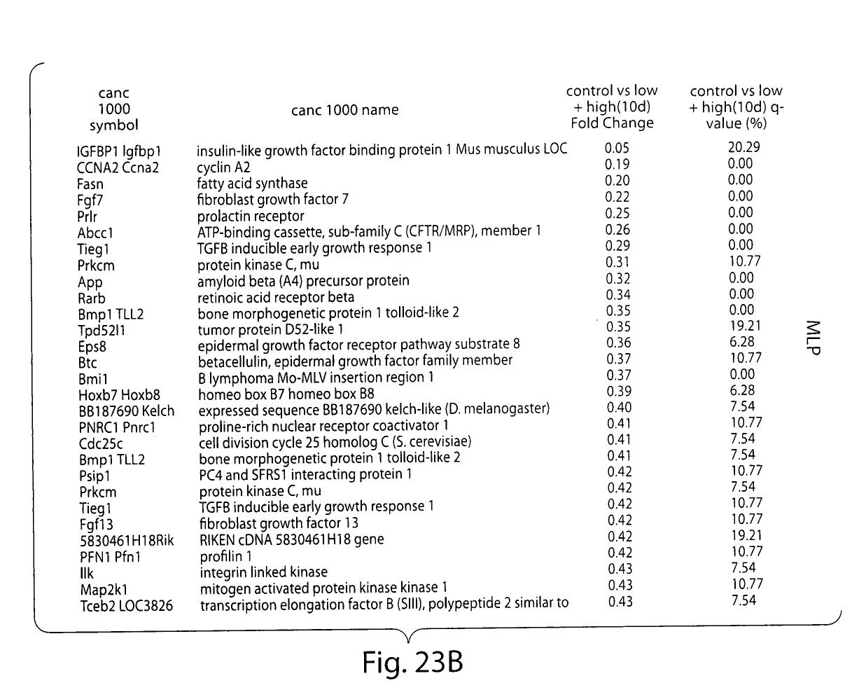

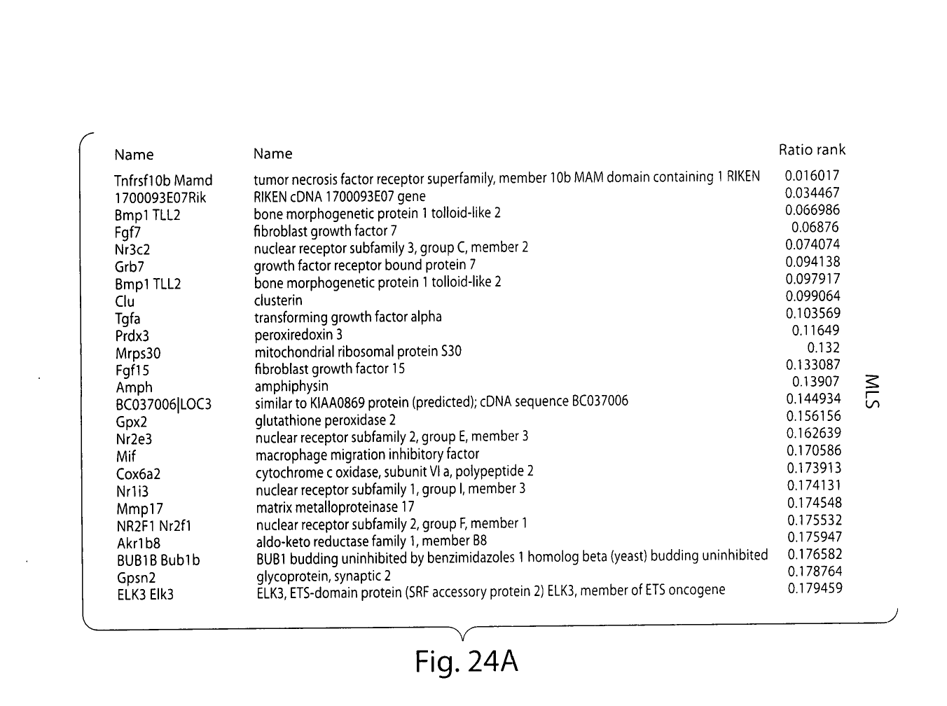

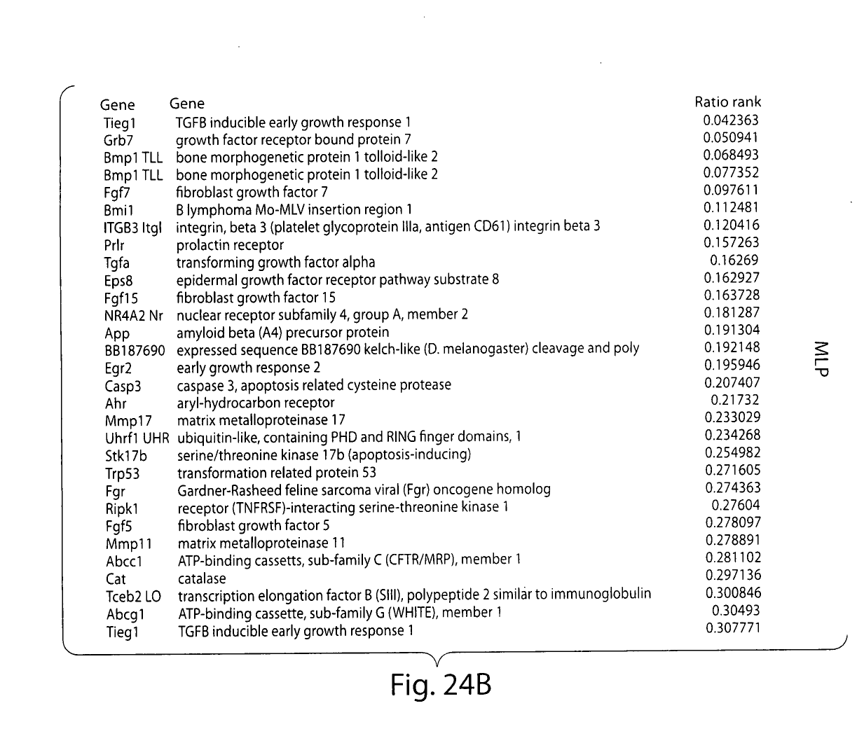

Additional, novel shRNAs that mediate doxorubicin resistance were identified using the microarray method, as shown in the lists of treatment-enriched shRNAs in FIGS. 21 and 22. Additionally, shRNAs targeting the genes that are listed in FIGS. 23 and 24 caused doxorubicin sensitization, suggesting that the down-regulation of the genes listed in FIGS. 23 and 24 may cause doxorubicin sensitization.

### Example 11

### Skp2 Down-regulation as a Novel Mechanism of Multidrug Resistance

sh-Skp2-688 RNA caused substantial doxorubicin resistance, comparable to the effect of Top2A, Chk2 and p53 shRNAs. Skp2 is an F-box protein that controls substrate specificity of SCF E3 ubiquitin ligase complexes, targeting, via ubiquitination, a variety of substrates for proteolytic degradation. The most important Skp2 target is considered to be p27^{KIP1}, a cyclin-dependent kinase inhibitor whose destruction is required for cell cycle progression (Nakayama and Nakayama, 2006). shSkp2-688 causes reproducible multidrug resistance: It protected against doxorubicin both in Eμ-Myc Arf^{-/-} lymphoma cells and E1-Myc p53^{-/-} lymphoma cells and also protected against camptothecin (data not shown).

### Example 12

### Bmi1 Down-Regulation or Inhibition as a Novel Method for Doxorubicin Sensitization

As demonstrated above via microarrays and subsequent validation, shBmi1-1741 caused doxorubicin sensitivity.

Bmi1 is a polycomb group protein and has been shown to support normal stem cell proliferation via its putative stem cell factor function (Park et al., 2003). As a supposed oncogene, it can cooperate with Myc in tumourigenesis (van Lohuizen et al., 1991), perhaps via transcriptional repression of the INK4A-ARF locus (Jacobs et al., 1999) and can also lead to cellular immortalisation (Dimri et al., 2002).

### REFERENCES

Aas, T., Borresen, A. L., Geisler, S., Smith-Sorensen, B., Johnsen, H., Varhaug, J. E., Akslen, L. A., and Lonning, P. E. (1996). Specific P53 mutations are associated with de novo resistance to doxorubicin in breast cancer patients. Nat. Med. 2, 811-814.
Akimitsu, N., Adachi, N., Hirai, H., Hossain, M. S., Hamamoto, H., Kobayashi, M., Aratani, Y., Koyama, H., and Sekimizu, K. (2003). Enforced cytokinesis without complete nuclear division in embryonic cells depleting the activity of DNA topoisomerase IIalpha. Genes Cells 8, 393-402.
Andoh, T. and Ishida, R. (1998). Catalytic inhibitors of DNA topoisomerase II. Biochim. Biophys. Acta 1400, 155-171.
Arriola, E., Moreno, A., Varela, M., Serra, J. M., Falo, C., Benito, E., and Escobedo, A. P. (2006). Predictive value of HER-2 and Topoisomerase IIalpha in response to primary doxorubicin in breast cancer. Eur. J. Cancer 42, 2954-2960.
Dickins, R. A., Hemann, M. T., Zilfou, J. T., Simpson, D. R., Ibarra, I., Hannon, G. J., and Lowe, S. W. (2005). Probing tumor phenotypes using stable and regulated synthetic microRNA precursors. Nat. Genet. 37, 1289-1295.
Fortune, J. M. and Osheroff, N. (2000). Topoisomerase II as a target for anticancer drugs: when enzymes stop being nice. Prog. Nucleic Acid Res. Mol. Biol. 64, 221-253.
Gudkov, A. V., Zelnick, C. R., Kazarov, A. R., Thimmapaya, R., Suttle, D. P., Beck, W. T., and Roninson, I. B. (1993). Isolation of genetic suppressor elements, inducing resistance to topoisomerase II-interactive cytotoxic drugs, from human topoisomerase II cDNA. Proc. Natl. Acad. Sci. U.S.A 90, 3231-3235.
Gupta, M., Fan, S., Zhan, Q., Kohn, K. W., O'Connor, P. M., and Pommier, Y. (1997). Inactivation of p53 increases the cytotoxicity of camptothecin in human colon HCT116 and breast MCF-7 cancer cells. Clin. Cancer Res. 3, 1653-1660.
Hirao, A., Cheung, A., Duncan, G., Girard, P. M., Elia, A. J., Wakeham, A., Okada, H., Sarkissian, T., Wong, J. A., Sakai, T., de Stanchina, E., Bristow, R. G., Suda, T., Lowe, S. W., Jeggo, P. A., Elledge, S. J., and Mak, T. W. (2002). Chk2 is a tumor suppressor that regulates apoptosis in both an ataxia telangiectasia mutated (ATM)-dependent and an ATM-independent manner. Mol. Cell Biol. 22, 6521-6532.
Hsiang, Y. H., Hertzberg, R., Hecht, S., and Liu, L. F. (1985). Camptothecin induces protein-linked DNA breaks via mammalian DNA topoisomerase I. J. Biol. Chem. 260, 14873-14878.
Hsiang, Y. H. and Liu, L. F. (1988). Identification of mammalian DNA topoisomerase I as an intracellular target of the anticancer drug camptothecin. Cancer Res. 48, 1722-1726.
Hu, T., Sage, H., and Hsieh, T. S. (2002). ATPase domain of eukaryotic DNA topoisomerase II. Inhibition of ATPase activity by the anti-cancer drug bisdioxopiperazine and ATP/ADP-induced dimerization. J. Biol. Chem. 277, 5944-5951.
Hu, X., Su, F., Qin, L., Jia, W., Gong, C., Yu, F., Guo, J., and Song, E. (2006). Stable RNA interference of ErbB-2 gene synergistic with epirubicin suppresses breast cancer growth in vitro and in vivo. Biochem. Biophys. Res. Commun. 346, 778-785.
Lowe, S. W., Bodis, S., McClatchey, A., Remington, L., Ruley, H. E., Fisher, D. E., Housman, D. E., and Jacks, T. (1994). p53 status and the efficacy of cancer therapy in vivo. Science 266, 807-810.
Lowe, S. W., Ruley, H. E., Jacks, T., and Housman, D. E. (1993). p53-dependent apoptosis modulates the cytotoxicity of anticancer agents. Cell 74, 957-967.
Morham, S. G., Kluckman, K. D., Voulomanos, N., and Smithies, O. (1996). Targeted disruption of the mouse topoisomerase I gene by camptothecin selection. Mol. Cell Biol. 16, 6804-6809.
Mueller, R. E., Parkes, R. K., Andrulis, I., and O'Malley, F. P. (2004). Amplification of the TOP2A gene does not predict high levels of topoisomerase II alpha protein in human breast tumor samples. Genes Chromosomes. Cancer 39, 288-297.
Mukhopadhyay, U. K., Senderowicz, A. M., and Ferbeyre, G. (2005). RNA silencing of checkpoint regulators sensitizes p53-defective prostate cancer cells to chemotherapy while sparing normal cells. Cancer Res. 65, 2872-2881.
Nitiss, J. and Wang, J. C. (1988). DNA topoisomerase-targeting antitumor drugs can be studied in yeast. Proc. Natl. Acad. Sci. U.S.A 85, 7501-7505.
Pang, E., Hu, Y., Chan, K. Y., Lai, P. B., Squire, J. A., Macgregor, P. F., Beheshti, B., Albert, M., Leung, T. W., and Wong, N. (2005). Karyotypic imbalances and differential gene expressions in the acquired doxorubicin resistance of hepatocellular carcinoma cells. Lab Invest 85, 664-674.
Schmitt, C. A., McCurrach, M. E., de Stanchina, E., Wallace-Brodeur, R. R., and Lowe, S. W. (1999). INK4a/ARF mutations accelerate lymphomagenesis and promote chemoresistance by disabling p53. Genes Dev. 13, 2670-2677.
Schmitt C A, Fridman J S, Yang M, Lee S, Baranov E, Hoffman R M, Lowe S W. A senescence program controlled by p53 and p161NK4a contributes to the outcome of cancer therapy. Cell, 2002 May 3; 109(3):335-46.
Silva, J. M., Li, M. Z., Chang, K., Ge, W., Golding, M. C., Rickles, R. J., Siolas, D., Hu, G., Paddison, P. J., Schlabach, M. R., Sheth, N., Bradshaw, J., Burchard, J., Kulkarni, A., Cavet, G., Sachidanandam, R., McCombie, W. R., Cleary, M. A., Elledge, S. J., and Hannon, G. J. (2005). Second-generation shRNA libraries covering the mouse and human genomes. Nat. Genet. 37, 1281-1288.
Takai, H., Naka, K., Okada, Y., Watanabe, M., Harada, N., Saito, S., Anderson, C. W., Appella, E., Nakanishi, M., Suzuki, H., Nagashima, K., Sawa, H., Ikeda, K., and Motoyama, N. (2002). Chk2-deficient mice exhibit radioresistance and defective p53-mediated transcription. EMBO J. 21, 5195-5205.
Tanner, M., Isola, J., Wiklund, T., Erikstein, B., Kellokumpu-Lehtinen, P., Malmstrom, P., Wilking, N., Nilsson, J., and Bergh, J. (2006). Topoisomerase IIalpha gene amplification predicts favorable treatment response to tailored and dose-escalated anthracycline-based adjuvant chemotherapy in HER-2/neu-amplified breast cancer: Scandinavian Breast Group Trial 9401. J. Clin. Oncol. 24, 2428-2436.
Villman, K., Sjostrom, J., Heikkila, R., Hultbom, R., Malmstrom, P., Bengtsson, N. O., Soderberg, M., Saksela, E., and Blomqvist, C. (2006). TOP2A and HER2 gene amplification as predictors of response to anthracycline treatment in breast cancer. Acta Oncol. 45, 590-596.
Wang, Y., Zhu, S., Cloughesy, T. F., Liau, L. M., and Mischel, P. S. (2004). p53 disruption profoundly alters the response of human glioblastoma cells to DNA topoisomerase I inhibition. Oncogene 23, 1283-1290.
Yasui, K., Mihara, S., Zhao, C., Okamoto, H., Saito-Ohara, F., Tomida, A., Funato, T., Yokomizo, A., Naito, S., Imoto, I., Tsuruo, T., and Inazawa, J. (2004). Alteration in copy numbers of genes as a mechanism for acquired drug resistance. Cancer Res. 64, 1403-1410.
Zhou, B. B. and Bartek, J. (2004). Targeting the checkpoint kinases: chemosensitization versus chemoprotection. Nat. Rev. Cancer 4, 216-225.
Li, M. Z. and Elledge, S. J. (2005). MAGIC, an in vivo genetic method for the rapid construction of recombinant DNA molecules. Nat. Genet. 37, 311-319.
Witt, A. E., Hines, L. M., Collins, N. L., Hu, Y., Gunawardane, R. N., Moreira, D., Raphael, J., Jepson, D., Koundinya, M., Rolfs, A., Taron, B., Isakoff, S. J., Brugge, J. S., and LaBaer, J. (2006). Functional proteomics approach to investigate the biological activities of cDNAs implicated in breast cancer. J. Proteome. Res. 5, 599-610.
Brummelkamp, T. R., Nijman, S. M., Dirac, A. M., and Bernards, R. (2003). Loss of the cylindromatosis tumour suppressor inhibits apoptosis by activating NF-kappaB. Nature 424, 797-801.
Dimri, G. P., Martinez, J. L., Jacobs, J. J., Keblusek, P., Itahana, K., van Lohuizen, M., Campisi, J., Wazer, D. E., and Band, V. (2002). The Bmi-1 oncogene induces telomerase activity and immortalizes human mammary epithelial cells. Cancer Res. 62, 4736-4745.
Farmer, H., McCabe, N., Lord, C. J., Tutt, A. N., Johnson, D. A., Richardson, T. B., Santarosa, M., Dillon, K. J., Hickson, I., Knights, C., Martin, N. M., Jackson, S. P., Smith, G. C., and Ashworth, A. (2005). Targeting the DNA repair defect in BRCA mutant cells as a therapeutic strategy. Nature 434, 917-921.
Gil, J., Bernard, D., Martinez, D., and Beach, D. (2004). Polycomb CBX7 has a unifying role in cellular lifespan. Nat. Cell Biol. 6, 67-72.
Glinsky, G. V., Berezovska, O., and Glinskii, A. B. (2005). Microarray analysis identifies a death-from-cancer signature predicting therapy failure in patients with multiple types of cancer. J. Clin. Invest 115, 1503-1521.
Jacobs, J. J., Scheijen, B., Voncken, J. W., Kieboom, K., Berns, A., and van Lohuizen, M. (1999). Bmi-1 collaborates with c-Myc in tumorigenesis by inhibiting c-Myc-induced apoptosis via INK4a/ARF. Genes Dev. 13, 2678-2690.
Liu, L., Andrews, L. G., and Tollefsbol, T. O. (2006). Loss of the human polycomb group protein BMI1 promotes cancer-specific cell death. Oncogene 25, 4370-4375.
Nakayama, K., Nagahama, H., Minamishima, Y. A., Miyake, S., Ishida, N., Hatakeyama, S., Kitagawa, M., Iemura, S., Natsume, T., and Nakayama, K. I. (2004). Skp2-mediated degradation of p27 regulates progression into mitosis. Dev. Cell 6, 661-672.
Nakayama, K. I. and Nakayama, K. (2005). Regulation of the cell cycle by SCF-type ubiquitin ligases. Semin. Cell Dev. Biol. 16, 323-333.
Nakayama, K. I. and Nakayama, K. (2006). Ubiquitin ligases: cell-cycle control and cancer. Nat. Rev. Cancer 6, 369-381.
Ngo, V. N., Davis, R. E., Lamy, L., Yu, X., Zhao, H., Lenz, G., Lam, L. T., Dave, S., Yang, L., Powell, J., and Staudt, L. M. (2006). A loss-of-function RNA interference screen for molecular targets in cancer. Nature 441, 106-110.
Oosterkamp, H. M., Neering, H., Nijman, S. M., Dirac, A. M., Mooi, W. J., Bernards, R., and Brummelkamp, T. R. (2006). An evaluation of the efficacy of topical application of salicylic acid for the treatment of familial cylindromatosis. Br. J. Dermatol. 155, 182-185.
Park, I. K., Qian, D., Kiel, M., Becker, M. W., Pihalja, M., Weissman, I. L., Morrison, S. J., and Clarke, M. F. (2003). Bmi-1 is required for maintenance of adult self-renewing haematopoietic stem cells. Nature 423, 302-305.
van Lohuizen, M., Verbeek, S., Scheijen, B., Wientjens, E., van der, G. H., and Berns, A. (1991). Identification of cooperating oncogenes in E mu-myc transgenic mice by provirus tagging. Cell 65, 737-752.
Yokoi, S., Yasui, K., Mori, M., Iizasa, T., Fujisawa, T., and Inazawa, J. (2004). Amplification and overexpression of SKP2 are associated with metastasis of non-small-cell lung cancers to lymph nodes. Am. J. Pathol. 165, 175-180.
Gros, P., Ben Neriah, Y. B., Croop, J. M. & Housman, D. E. (1986) Isolation and expression of a complementary DNA that confers multidrug resistance Nature 323, 728-731.
Chen, M. & Beck, W. T. (1995) DNA topoisomerase II expression, stability, and phosphorylation in two VM-26-resistant human leukemic CEM sublines Oncol. Res. 7, 103-111.
Mao, Y. et al. (1999) Mutations of human topoisomerase II alpha affecting multidrug resistance and sensitivity Biochemistry 38, 10793-10800.
Shah, N. P. et al. (2002) Multiple BCR-ABL kinase domain mutations confer polyclonal resistance to the tyrosine kinase inhibitor imatinib (ST1571) in chronic phase and blast crisis chronic myeloid leukemia Cancer Cell 2, 117-125.
Hannon, G. J. (2002) RNA interference Nature 418, 244-251.
Brummelkamp, T. R. et al. (2006) An shRNA barcode screen provides insight into cancer cell vulnerability to MDM2 inhibitors Nat. Chem. Biol. 2, 202-206.
Berns, K. et al. (2007) A functional genetic approach identifies the PI3K pathway as a major determinant of trastuzumab resistance in breast cancer Cancer Cell 12, 395-402.
Whitehurst, A. W. et al. (2007) Synthetic lethal screen identification of chemosensitizer loci in cancer cells Nature 446, 815-819.
Iorns, E., Lord, C. J., Turner, N. & Ashworth, A. (2007) Utilizing RNA interference to enhance cancer drug discovery Nat. Rev. Drug Discov. 6, 556-568.
Schmitt, C. A. et al. (2002) Dissecting p53 tumor suppressor functions in vivo Cancer Cell 1, 289-298.
Hemann, M. T. et al. (2004) Suppression of tumorigenesis by the p53 target PUMA Proc. Natl. Acad. Sci. U.S.A 101, 9333-9338.
Hemann, M. T. et al. (2003) An epi-allelic series of p53 hypomorphs created by stable RNAi produces distinct tumor phenotypes in vivo Nat. Genet. 33, 396-400.
Schmitt, C. A. et al. (2002) A senescence program controlled by p53 and p16INK4a contributes to the outcome of cancer therapy Cell 109, 335-346.
Sherr, C. J. (2001) The INK4a/ARF network in tumour suppression Nat. Rev. Mol. Cell Biol. 2, 731-737
Yu, Q., Rose, J. H., Zhang, H. & Pommier, Y. (2001) Antisense inhibition of Chk2/hCds1 expression attenuates DNA damage-induced S and G2 checkpoints and enhances apoptotic activity in HEK-293 cells FEBS Lett. 505, 7-12.
Hirao, A. et al. (2002) Chk2 is a tumor suppressor that regulates apoptosis in both an ataxia telangiectasia mutated (ATM)-dependent and an ATM-independent manner Mol. Cell Biol. 22, 6521-6532.
Wang, J. C. (2002) Cellular roles of DNA topoisomerases: a molecular perspective Nat. Rev. Mol. Cell Biol. 3, 430-440.
Strasser, A., Harris, A. W., Jacks, T. & Cory, S. (1994) DNA damage can induce apoptosis in proliferating lymphoid cells via p53-independent mechanisms inhibitable by Bcl-2 Cell 79, 329-339.
Miao, Z. H. et al. (2006) 4-nitroquinoline-1-oxide induces the formation of cellular topoisomerase I-DNA cleavage complexes Cancer Res. 66, 6540-6545.
Trigueros, S. & Roca, J. (2002) Failure to relax negative supercoiling of DNA is a primary cause of mitotic hyper-recombination in topoisomerase-deficient yeast cells J. Biol. Chem. 277, 37207-37211.
Uemura, T. & Yanagida, M. (1984) Isolation of type I and II DNA topoisomerase mutants from fission yeast: single and double mutants show different phenotypes in cell growth and chromatin organization EMBO J. 3, 1737-1744.
Rottenberg, S. et al. (2007) Selective induction of chemotherapy resistance of mammary tumors in a conditional mouse model for hereditary breast cancer Proc. Natl. Acad. Sci. U.S.A 104, 12117-12122.
Schmitt, C. A., Rosenthal, C. T. & Lowe, S. W. (2000) Genetic analysis of chemoresistance in primary murine lymphomas Nat. Med. 6, 1029-1035.
Mano, M. S., Rosa, D. D., de Azambuja, E., Ismael, G. F. & Durbecq, V. (2007) The 17q12-q21 amplicon: Her2 and topoisomerase-IIalpha and their importance to the biology of solid tumours Cancer Treat. Rev. 33, 64-77.
Jarvinen, T. A. & Liu, E. T. (2003) Topoisomerase IIalpha gene (TOP2A) amplification and deletion in cancer—more common than anticipated Cytopathology 14, 309-313.
Boonsong, A. et al. (2000) Characterization of the topoisomerase I locus in human colorectal cancer Cancer Genet. Cytogenet. 121, 56-60.
Brezinova, J. et al. (2005) Prognostic significance of del(20q) in patients with hematological malignancies Cancer Genet. Cytogenet. 160, 188-192.
Huesken, D. et al. (2005) Design of a genome-wide siRNA library using an artificial neural network Nat. Biotechnol. 23, 995-1001.

The practice of the various aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Current Protocols in Molecular Biology, by Ausubel et al., Greene Publishing Associates (1992, and Supplements to 2003); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W.H. Freeman & Co., New York (2000); Griffiths et al., Introduction to Genetic Analysis, 7th ed., W.H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All patents, patent applications and references cited herein are incorporated in their entirety by reference.

Those skilled in the art will recognize, or be able to ascertain using no more than routine experimentation, many equivalents to the specific embodiments of the invention described herein. Such equivalents are intended to be encompassed by the following embodiments.

What is claimed is:

1. A method for identifying a gene whose down-regulation in a cancer cell results in the cancer cell's resistance to a chemotherapeutic agent, comprising:
providing a library of RNA interference (RNAi) molecules, wherein each of said molecules inhibits expression of a target mammalian gene;
transfecting a plurality of mammalian cells with the library wherein said molecules are expressed;
treating the transfected cells with the chemotherapeutic agent; and
identifying an RNAi molecule that increases the survival of said transfected cells as compared to a control, wherein the target gene of the transfected RNAi molecule is a gene whose down-regulation in a cancer cell results in the cancer cell's resistance to said agent.

2. The method of claim 1, wherein said library comprises RNAi molecules that inhibit expression of genes known to be down-regulated in human cancers.

3. The method of claim 1, wherein said library comprises RNAi molecules that inhibit expression of genes known to be up-regulated in human cancers.

4. The method of claim 1, wherein said RNAi molecule is a small hairpin RNA molecule (shRNA).

5. The method of claim 1, wherein the chemotherapeutic agent targets topoisomerase 1 (TOP1).

6. The method of claim 5, wherein the agent is camptothecin or irinotecan.

7. The method of claim 1, wherein the chemotherapeutic agent targets topoisomerase 2A (TOP2A).

8. The method of claim 7, wherein the agent is doxorubincin.

9. The method of claim 1, wherein said RNAi molecule is identified using polymerase chain reaction (PCR).

10. The method of claim 1, wherein said RNAi molecule is identified using microarray.

11. A method for identifying a gene whose down-regulation in a cancer cell results in the cancer cell's sensitivity to a chemotherapeutic agent, comprising:
providing an RNAi molecule against a candidate gene;
transfecting a plurality of mammalian cells with the RNAi molecule wherein the RNAi molecule is expressed;
treating the transfected cells and control cells with the chemotherapeutic agent; and
monitoring survival of the treated transfected cells and the treated control cells,
wherein decreased survival of the transfected cells as compared control cells indicates that the candidate gene is a gene whose down-regulation in a cancer cell increases the cancer cell's sensitivity to said agent.

12. The method of claim 11, wherein the chemotherapeutic agent targets topoisomerase 2A (TOP2A).

13. A method for identifying a gene whose down-regulation in a cancer cell results in the cancer cell's sensitivity to a chemotherapeutic agent, comprising:
providing a library of RNA interference (RNAi) molecules, wherein each of said molecules inhibits expression of a target mammalian gene;
transfecting a plurality of mammalian cells with the library wherein said molecules are expressed;
treating the transfected cells with the chemotherapeutic agent;
identifying an RNAi molecule that decreases the survival of said transfected cells as compared to a control,
wherein the target gene of the transfected RNAi molecule is a gene whose down-regulation in a cancer cell results in the cancer cell's sensitivity to said agent.

14. The method of claim 13, wherein the chemotherapeutic agent targets TOP2A.

15. The method of claim 13, wherein said RNAi molecule is identified using microarray.

16. A method for identifying an agent that enhances the effectiveness of a cancer treatment with a TOP2-targeting chemotherapeutic agent, comprising:
contacting a mammalian cell with a candidate agent; and
comparing the expression or activity level of TOP1 of the treated cells to a control,
wherein a decrease in said TOP1 level of the treated cells as compared a control indicates that the compound enhances the effectiveness of said treatment.

17. A method for identifying an agent that enhances the effectiveness of a cancer treatment with a TOP2-targeting chemotherapeutic agent, comprising:
contacting a mammalian cell with a candidate agent; and
comparing the expression or activity level of Bmi1 of the treated cells to a control,
wherein a decrease in said Bmi1 level of the treated cells as compared a control indicates that the compound enhances the effectiveness of said treatment.

18. A method for identifying a cancer patient who may benefit from a treatment with a TOP2-targeting chemotherapeutic agent, comprising
obtaining a cancer cell from the patient;
determining the expression or activity level of TOP1 in the cancer cell;
wherein a decrease in said TOP1 level in the cancer cell as compared to a control indicates that the patient may benefit from said treatment.

19. A method for identifying a cancer patient who may benefit from a treatment with a TOP2-targeting chemotherapeutic agent, comprising
obtaining a cancer cell from the patient;
determining the expression or activity level of Bmi1 in the cancer cell;
wherein a decrease in said Bmi1 level in the cancer cell as compared to a control indicates that the patient may benefit from said treatment.

20. The method of claim 18 or 19, wherein the cancer cell is from bladder cancer, breast cancer, colon cancer, kidney cancer, liver cancer, lung cancer, esophagus cancer, gall bladder cancer, ovarian cancer, pancreas cancer, stomach cancer, cervical cancer, thyroid cancer, prostate cancer, skin cancer, leukemia, B-cell lymphoma, T-cell lymphoma, Hodgkins lymphoma, non-Hodgkins lymphoma, hairy cell lymphoma, Burkett's lymphoma, fibrosarcoma, rhabdomyosarcoma, astrocytoma, neuroblastoma, glioma and schwannomas, melanoma, seminoma, teratocarcinoma, osteosarcoma, xenoderoma pigmentosum, keratoctanthoma, thyroid follicular cancer, or Kaposi's sarcoma.

21. The method of claim 20, wherein the cancer cell is from acute myelogenous leukemia.

22. A method for treating a cancer patient identified by the method of claim 18 or 19, comprising administering to said patient a TOP2-targeting chemotherapeutic agent.

23. A method for treating a cancer patient, comprising administering to the patient a TOP1 inhibitor that down-regulates the expression or activity of TOP1, and a TOP2A-targeting chemotherapeutic agent.

24. The method of claim 23, wherein the TOP1 inhibitor is an RNAi molecule that inhibits expression of TOP1.

25. A method for treating a cancer patient, comprising administering to the patient a Bmi1 inhibitor that down-regulates the expression or activity of Bmi1, and a TOP2A-targeting chemotherapeutic agent.

26. The method of claim 25, wherein the Bmi1 inhibitor is an RNAi molecule that inhibits expression of Bmi1.

27. The method of claim 24 or 26, wherein the RNAi molecule is an shRNA molecule.

28. The method of claim 24 or 26, wherein the RNAi is part of a viral vector.

29. The method of claim 28, wherein the viral vector is an adenoviral, lentiviral, or retroviral vector.

30. The method of claim 24 or 26, wherein the RNAi is administered systemically in a pharmaceutical preparation.

31. The method of claim 23, wherein the TOP2A-targeting agent is doxorubicin, etoposide, mitoxantrone, mAMSA, amonafide, batracylin or menadione.

32. A method for inhibiting a cancer cell growth, comprising contacting the cancer cell with a TOP1 inhibitor that down-regulates the expression or activity of TOP1, and a TOP2A-targeting chemotherapeutic agent.

33. The method of claim 32, wherein the TOP1 inhibitor is an RNAi that inhibits expression of TOP1.

34. A method for inhibiting a cancer cell growth, comprising contacting the cancer cell with a Bmi1 inhibitor that down-regulates the expression or activity of Bmi1, and a TOP2A-targeting chemotherapeutic agent.

35. The method of claim 34, wherein the Bmi1 inhibitor is an RNAi that inhibits expression of Bmi1.

36. The method of claim 33 or 35, wherein the RNAi molecule is an shRNA molecule.

37. The method of claim 33 or 35, wherein the RNAi is part of a viral vector.

38. The method of claim 37, wherein the viral vector is an adenoviral, lentiviral, or retroviral vector.

39. The method of claim 33 or 35, wherein the RNAi is administered systemically in a pharmaceutical preparation.

40. The method of claim 32, wherein the TOP2A-targeting agent is doxorubicin, etoposide, mitoxantrone, mAMSA, amonafide, batracylin or menadione.


SEQUENCE LISTING (1 sequence)

SEQ ID NO: 1 - DNA, 23 bp, Artificial Sequence
  Description of Artificial Sequence Synthetic primer
cccttgaacctcctcgttcgacc